SEQUENCE LISTING (50 sequences)

SEQ ID NO: 1 - protein, 20 aa, Artificial Sequence
  Synthetic peptide
GCSKELDLVSHHVRTKLDEL

SEQ ID NO: 2 - protein, 16 aa, Artificial Sequence
  Synthetic peptide
PDTGLYYDEYLKQVIC

SEQ ID NO: 3 - protein, 20 aa, Artificial Sequence
  Synthetic peptide
GCQGIPPSGPAGELKFEPHI

SEQ ID NO: 4 - protein, 20 aa, Artificial Sequence
  Synthetic peptide
VPIDIDKTKVQNIHPVESAC

SEQ ID NO: 5 - protein, 82 aa, Homo sapiens
VPIDIDKTKVQNIHPVESAKIEPPDTGLYYDEYLKQVIDVLETDKHFREKLQKADIEEIKSGRLSKELDLVSHHVRTKLDEL

SEQ ID NO: 6 - protein, 82 aa, Mus musculus
VPIDVDKTKVHNTEPVENARIEPPDTGLYYDEYLKQVIEVLETDPHFREKLQKADIEEIRSGRLSQELDLVSHKVRTRLDEL

SEQ ID NO: 7 - protein, 82 aa, Rattus norvegicus
VPIDVDKTKVHNVEPVESARIEPPDTGLYYDEYLKQVIEVLETDPHFREKLQKADIEEIRSGRLSQELDLVSHKVRTRLDEL

SEQ ID NO: 8 - protein, 420 aa, Homo sapiens
MRWRTILLQYCFLLITCLLTALEAVPIDIDKTKVQNIHPVESAKIEPPDTGLYYDEYLKQVIDVLETDKHFREKLQKADIEEIKSGRLSKELDLVSHHVRTKLDELKRQEVGRLRMLIKAKLDSLQDIGMDHQALLKQFDHLNHLNPDKFESTDLDMLIKAATSDLEHYDKTRHEEFKKYEMMKEHERREYLKTLNEEKRKEEESKFEEMKKKHENHPKVNHPGSKDQLKEVWEETDGLDPNDFDPKTFFKLHDVNSDGFLDEQELEALFTKELEKVYDPKNEEDDMVEMEEERLRMREHVMNEVDTNKDRLVTLEEFLKATEKKEFLEPDSWETLDQQQFFTEEELKEYENIIALQENELKKKADELQKQKEELQRQHDQLEAQKLEYHQVIQQMEQKKLQQGIPPSGPAGELKFEPHI

SEQ ID NO: 9 - protein, 396 aa, Homo sapiens
VPIDIDKTKVQNIHPVESAKIEPPDTGLYYDEYLKQVIDVLETDKHFREKLQKADIEEIKSGRLSKELDLVSHHVRTKLDELKRQEVGRLRMLIKAKLDSLQDIGMDHQALLKQFDHLNHLNPDKFESTDLDMLIKAATSDLEHYDKTRHEEFKKYEMMKEHERREYLKTLNEEKRKEEESKFEEMKKKHENHPKVNHPGSKDQLKEVWEETDGLDPNDFDPKTFFKLHDVNSDGFLDEQELEALFTKELEKVYDPKNEEDDMVEMEEERLRMREHVMNEVDTNKDRLVTLEEFLKATEKKEFLEPDSWETLDQQQFFTEEELKEYENIIALQENELKKKADELQKQKEELQRQHDQLEAQKLEYHQVIQQMEQKKLQQGIPPSGPAGELKFEPHI

SEQ ID NO: 10 - protein, 420 aa, Mus musculus
MRWRIIQVQYCFLLVPCTLTALEAVPIDVDKTKVHNTEPVENARIEPPDTGLYYDEYLKQVIEVLETDPHFREKLQKADIEEIRSGRLSQELDLVSHKVRTRLDELKRQEVGRLRMLIKAKLDALQDTGMNHHLLLKQFEHLNHQNPNTFESRDLDMLIKAATADLEQYDRTRHEEFKKYEMMKEHERREYLKTLSEEKRKEEESKFEEMKRKHEDHPKVNHPGSKDQLKEVWEETDGLDPNDFDPKTFFKLHDVNNDGFLDEQELEALFTRELEKVYNPQNAEDDMIEMEEERLRMREHVMSEIDNNKDRLVTLEEFLRATEKKEFLEPDSWETLDQQQLFTEDELKEYESIIAIQENELKKRAEELQKQKEDLQRQHDHLEAQKQEYHQAVQHLEQKKLQQGIAPSGPAGELKFEPHT

SEQ ID NO: 11 - protein, 395 aa, Mus musculus
PIDVDKTKVHNTEPVENARIEPPDTGLYYDEYLKQVIEVLETDPHFREKLQKADIEEIRSGRLSQELDLVSHKVRTRLDELKRQEVGRLRMLIKAKLDALQDTGMNHHLLLKQFEHLNHQNPNTFESRDLDMLIKAATADLEQYDRTRHEEFKKYEMMKEHERREYLKTLSEEKRKEEESKFEEMKRKHEDHPKVNHPGSKDQLKEVWEETDGLDPNDFDPKTFFKLHDVNNDGFLDEQELEALFTRELEKVYNPQNAEDDMIEMEEERLRMREHVMSEIDNNKDRLVTLEEFLRATEKKEFLEPDSWETLDQQQLFTEDELKEYESIIAIQENELKKRAEELQKQKEDLQRQHDHLEAQKQEYHQAVQHLEQKKLQQGIAPSGPAGELKFEPHT

SEQ ID NO: 12 - protein, 420 aa, Rattus norvegicus
MRWRTIQARYCFLLVPCVLTALEAVPIDVDKTKVHNVEPVESARIEPPDTGLYYDEYLKQVIEVLETDPHFREKLQKADIEEIRSGRLSQELDLVSHKVRTRLDELKRQEVGRLRMLIKAKLDALQDTGMNHHLLLKQFEHLNHQNPDTFESKDLDMLIKAATADLEQYDRTRHEEFKKYEMMKEHERREYLKTLSEEKRKEEEAKFAEMKRKHEDHPKVNHPGSKDQLKEVWEETDGLDPNDFDPKTFFKLHDVNNDGFLDEQELEALFTKELDKVYNPQNAEDDMIEMEEERLRMREHVMNEIDNNKDRLVTLEEFLRATEKKEFLEPDSWETLDQQQLFTEEELKEYESIIAIQESELKKKADELQKQKEELQRQHDHLEAQKQEYQQAVQQLEQKKFQQGIAPSGPAGELKFEPHT

SEQ ID NO: 13 - protein, 396 aa, Rattus norvegicus
VPIDVDKTKVHNVEPVESARIEPPDTGLYYDEYLKQVIEVLETDPHFREKLQKADIEEIRSGRLSQELDLVSHKVRTRLDELKRQEVGRLRMLIKAKLDALQDTGMNHHLLLKQFEHLNHQNPDTFESKDLDMLIKAATADLEQYDRTRHEEFKKYEMMKEHERREYLKTLSEEKRKEEEAKFAEMKRKHEDHPKVNHPGSKDQLKEVWEETDGLDPNDFDPKTFFKLHDVNNDGFLDEQELEALFTKELDKVYNPQNAEDDMIEMEEERLRMREHVMNEIDNNKDRLVTLEEFLRATEKKEFLEPDSWETLDQQQLFTEEELKEYESIIAIQESELKKKADELQKQKEELQRQHDHLEAQKQEYQQAVQQLEQKKFQQGIAPSGPAGELKFEPHT

SEQ ID NO: 14 - protein, 435 aa, Homo sapiens
VPLERGAPNKEETPATESPDTGLYYHRYLQEVIDVLETDGHFREKLQAANAEDIKSGKLSRELDFVSHHVRTKLDELKRQEVSRLRMLLKAKMDAEQDPNVQVDHLNLLKQFEHLDPQNQHTFEARDLELLIQTATRDLAQYDAAHHEEFKRYEMLKEHERRRYLESLGEEQRKEAERKLEEQQRRHREHPKVNVPGSQAQLKEVWEELDGLDPNRFNPKTFFILHDINSDGVLDEQELEALFTKELEKVYDPKNEEDDMREMEEERLRMREHVMKNVDTNQDRLVTLEEFLASTQRKEFGDTGEGWETVEMHPAYTEEELRRFEEELAAREAELNAKAQRLSQETEALGRSQGRLEAQKRELQQAVLHMEQRKQQQQQQQGHKAPAAHPEGQLKFHPDTDDVPVPAPAGDQKEVDTSEKKLLERLPEVEVPQHL

SEQ ID NO: 15 - protein, 434 aa, Mus musculus
VPVDRAAPPQEDSQATETPDTGLYYHRYLQEVINVLETDGHFREKLQAANAEDIKSGKLSQELDFVSHNVRTKLDELKRQEVSRLRMLLKAKMDAKQEPNLQVDHMNLLKQFEHLDPQNQHTFEARDLELLIQTATRDLAQYDAAHHEEFKRYEMLKEHERRRYLESLGEEQRKEAERKLQEQQRRHREHPKVNVPGSQAQLKEVWEELDGLDPNRFNPKTFFILHDINSDGVLDEQELEALFTKELEKVYDPKNEEDDMREMEEERLRMREHVMKNVDTNQDRLVTLEEFLASTQRKEFGDTGEGWKTVEMSPAYTEEELKRFEEELAAREAELNARAQRLSQETEALGRSQDRLEAQKRELQQAVLQMEQRKQQLQEQSAPPSKPDGQLQFRADTDDAPVPAPAGDQKDVPASEKKVPEQPPELPQLDSQHL

SEQ ID NO: 16 - protein, 434 aa, Rattus norvegicus
VPVDRAAPHQEDNQATETPDTGLYYHRYLQEVINVLETDGHFREKLQAANAEDIKSGKLSQELDFVSHNVRTKLDELKRQEVSRLRMLLKAKMDAKQEPNLQVDHMNLLKQFEHLDPQNQHTFEARDLELLIQTATRDLAQYDAAHHEEFKRYEMLKEHERRRYLESLGEEQRKEAERKLQEQQRRHREHPKVNVPGSQAQLKEVWEELDGLDPNRFNPKTFFILHDINSDGVLDEQELEALFTKELEKVYDPKNEEDDMREMEEERLRMREHVMKNVDTNQDRLVTLEEFLASTQRKEFGETAEGWKTVEMYPAYTEEELKRFEEELAAREAELNARAQRLSQETEALGRSQDRLEAQKRELQQAVLQMEQRKQQQQEQSAPPSQPDGQLQFRADTGDAPVPAPAGDQKDVPASEKKVPEQPPVLPQLDSQHL

SEQ ID NO: 17 - protein, 77 aa, Homo sapiens
VPLERGAPNKEETPATESPDTGLYYHRYLQEVIDVLETDGHFREKLQAANAEDIKSGKLSRELDFVSHHVRTKLDEL

SEQ ID NO: 18 - protein, 77 aa, Mus musculus
VPVDRAAPPQEDSQATETPDTGLYYHRYLQEVINVLETDGHFREKLQAANAEDIKSGKLSQELDFVSHNVRTKLDEL

SEQ ID NO: 19 - protein, 77 aa, Rattus norvegicus
VPVDRAAPHQEDNQATETPDTGLYYHRYLQEVINVLETDGHFREKLQAANAEDIKSGKLSQELDFVSHNVRTKLDEL

SEQ ID NO: 20 - DNA, 30 bp, Artificial Sequence
  Synthetic oligonucleotide primer
ggagataaaaattatttacctgcctgaaca

SEQ ID NO: 21 - DNA, 30 bp, Artificial Sequence
  Synthetic oligonucleotide primer
aaatatttattgagcagagaaaagggaagg

SEQ ID NO: 22 - DNA, 59 bp, Artificial Sequence
  Synthetic oligonucleotide primer
ggttccgcgggtctggttccgcgtggttctgtgcctattgatatagacaagacaaaagt

SEQ ID NO: 23 - DNA, 39 bp, Artificial Sequence
  Synthetic oligonucleotide primer
ggttgcggccgcttacagttcatcaagttttgtcctcac

SEQ ID NO: 24 - DNA, 38 bp, Artificial Sequence
  Synthetic oligonucleotide primer
ggttgcggccgcgactttaaatgtgtggctcaaacttc

SEQ ID NO: 25 - DNA, 20 bp, Artificial Sequence
  Synthetic oligonucleotide primer
tgctgctgctgctcctgctt

SEQ ID NO: 26 - DNA, 40 bp, Artificial Sequence
  Synthetic oligonucleotide primer
ggttgcggccgctcacagatgctggggcacctcaacctca

SEQ ID NO: 27 - DNA, 57 bp, Artificial Sequence
  Synthetic oligonucleotide primer
ggttccgcgggtctggttccgcgtggttctgtccccctggagcgaggggcgcccaac

SEQ ID NO: 28 - DNA, 39 bp, Artificial Sequence
  Synthetic oligonucleotide primer
ggttgcggccgctcagagctcatccagcttggtgcggac

SEQ ID NO: 29 - protein, 6 aa, Homo sapiens
LVPRGS

SEQ ID NO: 30 - DNA, 21 bp, Artificial Sequence
  Synthetic oligonucleotide primer
gcacgctgaccgctctggaag

SEQ ID NO: 31 - DNA, 26 bp, Artificial Sequence
  Synthetic oligonucleotide primer
ttgagtggagagaaaagaaaggatgt

SEQ ID NO: 32 - DNA, 56 bp, Artificial Sequence
  Synthetic oligonucleotide primer
ggttccgcgggtctggttccgcgtggttctgttcctatcgatgtggacaagaccaa

SEQ ID NO: 33 - DNA, 39 bp, Artificial Sequence
  Synthetic oligonucleotide primer
ggttgcggccgcttacagctcatccagtctcgtcctcac

SEQ ID NO: 34 - DNA, 39 bp, Artificial Sequence
  Synthetic oligonucleotide primer
ggttgcggccgcactttatgtgtgtggctcaaacttcag

SEQ ID NO: 35 - DNA, 58 bp, Artificial Sequence
  Synthetic oligonucleotide primer
ggttactagtggttctggtcatcaccatcaccatcactccgcgggtctggttccgcgt

SEQ ID NO: 36 - DNA, 20 bp, Artificial Sequence
  Synthetic oligonucleotide primer
accagccacgatgcctacct

SEQ ID NO: 37 - DNA, 23 bp, Artificial Sequence
  Synthetic oligonucleotide primer
cacactgtctgtgggaacctgag

SEQ ID NO: 38 - DNA, 58 bp, Artificial Sequence
  Synthetic oligonucleotide primer
ggttccgcgggtctggttccgcgtggttctgtgcccgtggaccgcgcagcacctcctc

SEQ ID NO: 39 - DNA, 39 bp, Artificial Sequence
  Synthetic oligonucleotide primer
ggttgcggccgcttagagctcatccagcttggttcggac

SEQ ID NO: 40 - DNA, 39 bp, Artificial Sequence
  Synthetic oligonucleotide primer
ggttgcggccgcttataaatgctgggaatccagctgtgg

SEQ ID NO: 41 - DNA, 24 bp, Artificial Sequence
  Synthetic oligonucleotide primer
ctctctgaagatgaggtggaggac

SEQ ID NO: 42 - DNA, 25 bp, Artificial Sequence
  Synthetic oligonucleotide primer
tgagtgaagagaaaagaagggatgt

SEQ ID NO: 43 - DNA, 58 bp, Artificial Sequence
  Synthetic oligonucleotide primer
cactccgcgggtctggttccgcgtggttctgttcctattgatgtggacaagaccaaag

SEQ ID NO: 44 - DNA, 40 bp, Artificial Sequence
  Synthetic oligonucleotide primer
ggttgcggccgcttacagttcatccagtctcgtcctcact

SEQ ID NO: 45 - DNA, 38 bp, Artificial Sequence
  Synthetic oligonucleotide primer
ggttgcggccgcgagaaaagaagggatgttgaaagatg

SEQ ID NO: 46 - DNA, 20 bp, Artificial Sequence
  Synthetic oligonucleotide primer
cgatgcctacctctgtgccc

SEQ ID NO: 47 - DNA, 25 bp, Artificial Sequence
  Synthetic oligonucleotide primer
ccatcactgtctgtgggaacttgag

SEQ ID NO: 48 - DNA, 53 bp, Artificial Sequence
  Synthetic oligonucleotide primer
ggttccgcgggtctggttccgcgtggttctgtgcctgtggaccgcgcagcacc

SEQ ID NO: 49 - DNA, 39 bp, Artificial Sequence
  Synthetic oligonucleotide primer
ggttgcggccgcttagagctcatccagcttggtgcggac

SEQ ID NO: 50 - DNA, 38 bp, Artificial Sequence
  Synthetic oligonucleotide primer
ggttgcggccgcttataaatgctgagaatccagctgtg

(19) United States
(12) Patent Application Publication
(10) Pub. No.: US 2011/0110949 A1
(43) Pub. Date: May 12, 2011

(54) NESFATIN-1 SPECIFIC ANTIBODY AND THE USE THEREOF, AND NESFATIN SPECIFIC ANTIBODY AND THE USE THEREOF

(75) Inventors: Masatomo Mori, Maebashi-shi (JP); Hiroyuki Shimizu, Maebashi-shi (JP); Hiroshi Eguchi, Hino-shi (JP); Takashi Kawamura, Hino-shi (JP)

(73) Assignees: TEIJIN PHARMA LIMITED, Chiyoda-ku, Tokyo (JP); NATIONAL UNIVERSITY CORPORATION GUNMA UNIVERSITY, Maebashi-shi, Gunma (JP)

(21) Appl. No.: 12/674,902

(22) PCT Filed: Aug. 25, 2008
(86) PCT No.: PCT/JP2008/065622

§ 371 Date: Feb. 23, 2010

Related U.S. Application Data
(60) Provisional application No. 60/935,652, filed on Aug. 23, 2007.

(51) Int. Cl. A61K 39/395; C07K 16/00; C12N 5/16; G01N 33/566; A61P 3/00

(52) U.S. Cl. 424/139.1; 530/387.9; 435/331; 436/501

(57) ABSTRACT

The present application relates to an antibody performing an antigen-antibody reaction with Nesfatin-1, but not substantially performing the antigen-antibody reaction with Nesfatin and/or NucB1, and with the use of the antibody, an immunological detection method and a detection kit of Nesfatin-1, and further, an antibody performing an antigen-antibody reaction with Nesfatin, but not substantially performing the antigen-antibody reaction with NucB1, and an immunological detection method using the antibody, and a detection kit of Nesfatin comprising the antibody.

### FIELD OF THE INVENTION

The present invention relates to Nesfatin-1 specific antibody that was identified for the first time according to the present invention and the use thereof, and Nesfatin that was identified for the first time according to the present invention specific antibody and the use thereof.

### BACKGROUND OF THE INVENTION

Obesity is a state having excessive body weight (especially, white adipose tissues), and in general, classified by Body Mass Index (BMI) ≧25 kg/m² and further classified by a body fat percentage of 25% or more for adult males and 30% or more for adult females. In these days of the dietary habit loaded with high-fat foods and the lack of exercise, the percentage of the people classified into obese tends to increase. The results of National Nutrition Survey by the Ministry of Health, Labour and Welfare in 2000 indicate that males classified into the obese have definitely increased in comparison with that in the last decade and two decades, and around 30% of the males from 4.0 to 69 years old are classified into the obese. Further, in females, around 30% of the females from 60 to 69 years old are also classified into the obese.

Currently, health disorder (capable of being) associated with obesity other than the obesity itself is clinically a large issue, and forms a medical reason for the prevention or treatment of obesity. The Japan Society for the Study of Obesity defines adiposis as “a pathological condition complicating health disorder that is caused by or associated with obesity, or medically requiring weight reduction when the complication is clinically predicted” and advocates to treat adiposis as a disease. In “health disorder” herein described, in addition to type 2 diabetes mellitus and impaired glucose tolerance, hypertension, hyperlipemia, hyperuricemia, fatty liver, cardiovascular/cerebrovascular disease, sleep apnea syndrome, orthopedic disease such as osteoarthritis and the like, menstrual disorder and others are included (Yuji Matsuzawa, Nippon-Rinsho, Nippon Rinsho Co., Ltd., “Obesity” extra No. 6, Vol. 61, p 5-8, Jul. 28, 2003). In addition, it is reported that as a disease caused by obesity, malignant tumors are mentioned, and obesity is a risk factor for the onset of especially breast cancer, uterus cancer, colon cancer, kidney cancer, esophagus cancer, pancreas cancer, hepatic cancer and gallbladder cancer (Yuji Matsuzawa, Nippon-Rinsho, Nippon Rinsho Co., Ltd., “Obesity” extra No. 6, Vol. 61, p 5-8, Jul. 28, 2003; Abu-Abid et al., Journal of medicine (MSA), Vol. 33, No. 1-4, p 73-86, Jan. 1, 2002; and Nair et al., Hepatology (MSA), Vol. 36, No. 1, p 150-155, Jul. 1, 2002). Further, in recent years, a combined risk syndrome that is referred to as a metabolic syndrome and increases the risk of an arteriosclerotic disease (myocardial infarction, cerebral infarction and the like) has been proposed and has drawn the attention for the fact that 30% in total mortality is caused by cerebral vascular disorder and cardio vascular disorder in Japan. Therefore, the diagnostic criteria were established jointly by the Japan Society for the Study of Obesity, the Japan Atherosclerosis Society, the Japan Diabetes Society, the Japanese Society of Hypertension, the Japanese Circulation Society, the Japanese Society of Nephrology, the Japanese Society on Thrombosis and Hemostasis, and the Japanese Society of Internal Medicine and published at the press conference in the Japanese Society of Internal Medicine on 8th of April in 2005. According to the diagnostic criteria, a metabolic syndrome is diagnosed in the case of having two or more risks among the risks of impaired serum lipid (having either or both a triglyceride level of 150 mg/dL or more and/or an HDL cholesterol level of less than 40 mg/dL), high blood pressure (having either or both a systolic blood pressure of 130 mmHg or more and/or a diastolic blood pressure of 85 mmHg) and high blood sugar (a fasting blood sugar level of 110 mg/dL or more), in addition to having a waist circumference of 85 cm or more for males and 90 cm or more for females, while setting the visceral obesity (visceral fat accumulation) in the center of the risks (Journal of Japanese Society of Internal Medicine, Exploratory Committee for Diagnostic Criteria of Metabolic Syndrome, Vol. 94, April issue in 2005, p 794-809). There is also a report that, when the diagnostic criteria are applied, among the 290 adult males who had a complete medical checkup, while 61 males (21%) were diagnosed with adipositas, 27 males (9%) were diagnosed with metabolic syndrome and even 9 males (3%) were diagnosed with metabolic syndrome without being included in adipositas (Kazuo Takahashi, Yasushi Saito, Igaku no Ayumi, Vol. 213, No. 6, p 549-554, 2005).

As opposed to the obesity, excessive weight loss (what is called “skinny”) and decreased food intake (what is called “decreased appetite”) cause a problem as a factor of easy infection caused by reduced-biological defense (immune) response, hematopoietic disorder, amenorrhea or irregular menstruation, infertility, psychical disorder, peripheral nerve paralysis, hypotension, osteoporosis and the like. In general, when the BMI is <18.5 kg/m², or the body fat percentage is 10% or less for males and 15% or less for females, the males/females are classified into “skinny”. In the National Nutrition Survey by the Ministry of Health, Labour and Welfare in 2000, the percentage of the females having BMI <18.5 kg/m² has steadily increased between 20 and 39 years old during the last decade and two decades, and around 24% of the females between 20 and 29 years old are classified into “skinny”. This may be caused in young females by intentionally regulating the amount of food intake with a concern for their body shapes. However, in the case of anorexia nervosa (anorexia) and the like among the food intake abnormalities of central origin that occur frequently in this age group, the appetite itself is significantly decreased, and the nutritional status is compromised, as a result, the general debility may cause death. Further, as a disease causing a decrease of appetite and including the concept that is conventionally referred to as descensus ventriculi, gastric atony and neurotic gastritis, there is a disease referred to as “Functional dyspepsia”, and the disease is said to exhibit a symptom such as an early satiety sensation after eating, a loss of appetite and the like (Talley et al., Gut (England), Vol. 45, Suppl. 2, p 1137-1142, 1999). Furthermore, a factor causing a decreased appetite is exemplified by a cancer, an inflammatory disease, a decline in the function of pituitary gland, thyroid gland, adrenal gland and the like, after surgery, an extreme stress, and others. Under such conditions, by a decreased appetite persisting for a long time, wasting of the body is brought about.

In these circumstances, biological factors regulating food intake are recently actively studied and the relation between a factor such as leptin, adiponectin, ghrelin and the like and the food intake regulation is also studied. In recent years, as a substance relating to food intake and obesity, Nesfatin-1 is reported (Oh-I S. et al., Nature, 443 (7112):709-12, 2006), and which is expected as a novel factor involved in the food intake regulation and/or body weight regulation.

Nesfatin-1 is a peptide composed of the 82 amino acids spliced out from the protein composed of 420 amino acids and referred to known Nucleobindin 2 (NucB2), and as a result of examination, it was found that Nesfatin-1 is a peptide showing an action on food intake regulation by generating Nesfatin-1 from the NucB2 without having an action on food intake regulation (International Publication Number WO2006/137597). As described above, since Nesfatin-1 has part of the sequence of NucB2 and has a common amino acid composition to NucB2, it was considered that an antibody binding to only Nesfatin-1 was commonly significantly difficult to obtain without binding to the NucB2. Therefore, a method of detecting an active molecule in vivo, namely, Nesfatin-1, is required to separate NucB2 from Nesfatin-1 using another physical procedure and to detect by the antibody as described in International Publication Number WO2006/137597. This method was not considered to be a practical measurement system of Nesfatin-1 to use for diagnoses in clinical practice and the like in respect of complication of the detecting procedure, difficulty of controlling the efficiency of extraction and purification in separation process, and the like. In addition, it is also desired not to perform a cross-reaction with NucB1 since NucB2 has high homology with NucB1 belonging the same family thereof.

Further, it is shown that an antibody binding to both of NucB2 and Nesfatin-1 has an action of facilitating food intake by administering the antibody into a brain of rat. However, administration of a treatment agent directly into a brain is not practical when used as a practical treatment agent, and a medicament with the form administering via blood vessels and the like is desirable. At this time, in the case that a large level of NucB1 presents in blood and the like, when the antibody showing the cross-reaction with NucB1 is administered, the antibody is grabbed by the NucB1 before reaching the site where Nesfatin-1 presents and may not exert the effect of the antibody sufficiently.

### SUMMARY OF THE INVENTION

An object to be solved by the present invention is to provide an antibody performing an antigen-antibody reaction with Nesfatin-1, but not substantially performing the antigen-antibody reaction with Nesfatin (Nucleobindin 2 (NucB2)) or NucB1, or an antibody performing an antigen-antibody reaction with Nesfatin-1, but not substantially performing the antigen-antibody reaction with Nesfatin and NucB1, as well as an immunological detection method of Nesfatin-1 using the antibody and a detection kit of Nesfatin-1 comprising the antibody.

Further, another object to be solved by the present invention is to provide an antibody performing an antigen-antibody reaction with Nesfatin, but not substantially performing the antigen-antibody reaction with NucB1, as well as an immunological detection method of Nesfatin using the antibody and a detection kit of Nesfatin comprising the antibody.

The present inventors found that an antibody performing an antigen-antibody reaction with Nesfatin-1, but not substantially performing the antigen-antibody reaction with Nesfatin or NucB1; and an antibody performing an antigen-antibody reaction with Nesfatin-1, but not substantially performing the antigen-antibody reaction with Nesfatin and NucB1, were obtained by examining the antibodies obtained with the use of various types of peptides in Nesfatin-1 as an immunogen and by using a peptide composed of a specific sequences in Nesfatin-1 as an immunogen. Further, the present inventors found a method of immunologically detecting Nesfatin-1 with a high sensitivity by obtaining an antibody showing an antigen-antibody reaction with Nesfatin with a high sensitivity, and combining the antibody with a Nesfatin-1-specific antibody, and thus completed the present invention based on these findings.

That is, the present invention relates to the following.

(1) An antibody performing an antigen-antibody reaction with Nesfatin-1, but not substantially performing the antigen-antibody reaction with Nesfatin (antibody No. 4998, NAP40-2 and NAF7).
(2) The antibody according to (1), obtainable by using a peptide having the following amino acid sequence as an immunogen (antibody No. 4998).

NSF1-C18:
-Gly-Cys-Ser-Lys-Glu-Leu-Asp-Leu-Val-Ser-His-
His-Val-Arg-Thr-Lys-Leu-Asp-Glu-Leu

(3) An antibody performing an antigen-antibody reaction with Nesfatin-1, but not substantially performing the antigen-antibody reaction with NucB1 (antibody No. 4994, 5223, 6151, 6152, NAP40-2, NAE1, NAE3 and NAF11).
(4) The antibody according to (3), wherein the antibody is a monoclonal antibody and is produced by a hybridoma deposited under an accession number: FERM ABP-10881, FERM ABP-10882, FERM ABP-10883 or FERM ABP-10884 (antibody No. NAE1, NAF7, NAF11 and NAP40-2).
(5) An antibody performing an antigen-antibody reaction with Nesfatin-1, but not substantially performing the antigen-antibody reaction with Nesfatin and NucB1.
(6) The antibody according to (5), wherein the antibody is a monoclonal antibody and is produced by a hybridoma deposited under an accession number: FERM ABP-10882 or FERM ABP-10884 (antibody No. NAF7 and NAP40-2).
(7) An antibody performing an antigen-antibody reaction with Nesfatin, but not substantially performing the antigen-antibody reaction with Nesfatin-1 and NucB1 (4994 and NAD15).
(8) A hybridoma deposited under an accession number: FERM ABP-10881, FERM ABP-10882, FERM ABP-10883 or FERM ABP-10884.
(9) An immunological detection method of Nesfatin-1 comprising:

a step of bringing a test substance into contact with the antibody according to any of (1) to (6); and

a step of detecting an antibody performing an antigen-antibody reaction with the test substance.

(10) An immunological detection method of Nesfatin comprising:

a step of bringing a test substance into contact with the antibody according to any of (7); and

a step of detecting an antibody performing an antigen-antibody reaction with the test substance.

(11) An immunological detection method of Nesfatin-1 in a test substance, using two types of antibodies selected from the antibodies according to (1) to (6).
(12) The immunological detection method according to claim 11, comprising:

a step of bringing a test substance into contact with a primary antibody immobilized on a solid phase;

a step of bringing the test substance to which the primary antibody is bound into contact with a labeled secondary antibody; and then

a step of detecting a secondary antibody performing an antigen-antibody reaction with the test substance; and

wherein one of the primary antibody and the secondary antibody is the antibody according to any of (1), (2), (5) and (6); and

wherein the other of the primary antibody and the secondary antibody is the antibody according to any of (3), (4), (5) and (6).

(13) The immunological detection method according to (12), wherein

the primary antibody is the antibody (antibody No. 4998) according to (1) or (2), and

the secondary antibody is an antibody (antibody No. NAE1, or antibody No. NAF11) produced by a hybridoma deposited under an accession number: FERM ABP-10881 or FERM ABP-10883.
(14) The immunological detection method according to (12), wherein

the primary antibody is an antibody (antibody No. NAP40-2, or antibody No. NAF7) produced by a hybridoma deposited under an accession number: FERM ABP-10884 or FERM ABP-10882, and

the secondary antibody is the antibody (antibody No. 4998) according to (1) or (2).

(15) The immunological detection method according to any of (11) to (14), having a sensitivity discriminating between a test substance at a Nesfatin-1 concentration of less than 30 pM and a control substance at a Nesfatin-1 concentration of 0 pM.
(16) An immunological detection method of Nesfatin in a test substance, comprising:

a step of bringing a test substance into contact with a primary antibody immobilized on a solid phase;

a step of bringing the test substance to which the primary antibody is bound into contact with a labeled, secondary antibody; and then a step of detecting a secondary antibody performing an antigen-antibody reaction with the test substance; and

wherein one of the primary antibody and the secondary antibody is the antibody according to (7); and
wherein the other of the primary antibody and the secondary antibody is an antibody performing an antigen-antibody reaction with Nesfatin, but not substantially performing the antigen-antibody reaction with NucB1.
   (17) The immunological detection method according to (16), having a sensitivity discriminating between a test substance at a Nesfatin concentration of less than 30 pM and a control substance at a Nesfatin concentration of 0 pM.
   (18) The immunological detection method according to (16) or (17), wherein

the primary antibody is the antibody (antibody No. 4994, NAD15) according to (7), and the secondary antibody is an antibody (antibody No.NAE1, NAF11) produced by a hybridoma deposited under an accession number: FERM ABP-10881 or FERM ABP-10883.

(19) A detection kit of Nesfatin-1 used for the detection method according to any of (12) to (15), comprising:

a solid phase on which a primary antibody immobilized, and a labeled secondary antibody; and

wherein one of the primary antibody and the secondary antibody being the antibody according to any of (1), (2), (5) and (6); and

wherein the other of the primary antibody and the secondary antibody being the antibody according to any of (3), (4), (5) and (6).

(20) A detection kit of Nesfatin used for the detection method according to any of (16) to (18), comprising:

a solid phase on which a primary antibody immobilized, and a labeled secondary antibody; and

wherein one of the primary antibody and the secondary antibody being the antibody according to (7); and

wherein the other of the primary antibody and the secondary antibody being an antibody performing an antigen-antibody reaction with Nesfatin, but not substantially performing the antigen-antibody reaction with NucB1.

(21) An immunological Detection Method of Nesfatin-1, Comprising:

a step of mixing a labeled Nesfatin-1 standard substance with a test substance;

a step of bringing the antibody according to any of (1) to (6) into contact with the test substance mixed with the labeled Nesfatin-1 standard substance; and

a step of detecting a labeled Nesfatin-1 standard substance performing an antigen-antibody reaction with the antibody.

(22) The immunological detection method according to (21), wherein the antibody is the antibody according to (5) or (6).
(23) An immunological detection method of Nesfatin, comprising:

a step of mixing a labeled Nesfatin standard substance with a test substance;

a step of bringing an antibody performing an antigen-antibody reaction with Nesfatin, but not substantially performing the antigen-antibody reaction with NucB1 into contact with the test substance mixed with the labeled Nesfatin standard substance; and

a step of detecting a labeled Nesfatin standard substance performing an antigen-antibody reaction with the antibody.

(24) The immunological detection method according to (23), wherein the antibody is the antibody according to (7).
(25) A detection kit of Nesfatin-1 used for the detection method according to (21) or (22), comprising:

a labeled Nesfatin-1 standard substance and an antibody performing an antigen-antibody reaction with Nesfatin-1; and

wherein the antibody is the antibody according to any of (1) to (6).

(26) A detection kit of Nesfatin used for the detection method according to (23) to (24), comprising:

a labeled Nesfatin standard substance and an antibody performing an antigen-antibody reaction with Nesfatin; and

wherein the antibody is the antibody according to (7).

(27) A pharmaceutical composition for increased appetite and/or increased body weight gain, comprising the antibody according to any of (1) to (7).

### BRIEF EXPLANATION OF THE DRAWINGS

FIG. 1 is a schematic diagram showing the location of peptide (hNSF-N19, hNSF1-C18, hNSF-C18 or hNSF1-M15) used as an antigen in Nesfatin or Nesfatin-1. Further, the entire length of the polypeptide shown in FIG. 1 represents Nesfatin, SP represents signal peptide, and NAP1, NAP2 and NAP3 represent Nesfatin-1, Nesfatin-2 and Nesfatin-3, respectively.

FIG. 2 is a graph showing a reactivity with human Nesfatin (F-NAP) in the sandwitch ELISA using an anti-Nesfatin IgG (antibody No. 4994)-immobilized plate and a biotinylated antibody (antibody No. 4994, 6151, 6152 or 6153).

FIG. 3 is a graph showing a cross-reactivity with rat Nesfatin in the sandwitch ELISA using an anti-Nesfatin IgG (antibody No. 4994)-immobilized plate and a biotinylated antibody (antibody No. 6151 or 6152).

FIG. 4 is a graph showing a cross-reactivity with rat or mouse Nesfatin in the sandwitch ELISA using an anti-Nesfatin IgG (antibody No. 4994)-immobilized plate and a biotinylated antibody (antibody No. 6151 or 6152).

FIG. 5 is a graph showing a reactivity with human or rat Nesfatin-1 in the sandwitch ELISA using an anti-Nesfatin-1 IgG (antibody No. 6151)-immobilized plate and a biotinylated IgG (antibody No. 4998, 6151 or 6152).

FIG. 6 is a graph showing a reactivity with human or rat Nesfatin-1 in the sandwitch ELISA using an anti-Nesfatin-1 IgG (antibody No. 6152)-immobilized plate and a biotinylated IgG (antibody No. 4998, 6151 or 6152).

FIG. 7 is a graph showing a cross-reactivity with human Nesfatin or human NucB1-N77 in the sandwitch ELISA using an anti-Nesfatin-1 IgG (antibody No. 6152)-immobilized plate and a biotinylated IgG (antibody No. 6151 or 6152).

FIG. 8 is a graph showing a reactivity with human Nesfatin, human Nesfatin-1 or human NucB1-N77 in the sandwitch ELISA using an anti-Nesfatin-1 IgG (antibody No. 4998)-immobilized plate and a biotinylated IgG (antibody No. 6151 or 6152).

FIG. 9 is a graph showing a cross-reactivity with rat or mouse Nesfatin-1 in the sandwitch ELISA using an anti-Nesfatin-1 IgG (antibody No. 4998)-immobilized plate and a biotinylated antibody (antibody No. 6152).

FIG. 10 is a graph showing a reactivity with human Nesfatin-1 in the sandwitch ELISA using an anti-recombinant human Nesfatin-1 mouse monoclonal antibody (NAP40-2, NAP37 or NAP39)-immobilized plate and a biotinylated IgG (biotinylated antibody No. 6151).

FIG. 11 is a graph showing a reactivity with human Nesfatin-1 in the sandwitch ELISA using an anti-recombinant human Nesfatin-1 mouse monoclonal antibody (NAP40-2, NAP37 or NAP39)-immobilized plate and a biotinylated IgG (biotinylated antibody No. 6152).

FIG. 12 is a graph showing a reactivity with human Nesfatin-1 in the sandwitch ELISA using an anti-recombinant human Nesfatin-1 mouse monoclonal antibody (NAP40-2)-immobilized plate and a biotinylated IgG (biotinylated antibody No. 4998, biotinylated antibody No. 6151 or biotinylated antibody No. 6152).

FIG. 13 is a graph showing a reactivity with human Nesfatin-1 in the sandwitch ELISA using an anti-recombinant human Nesfatin-1 mouse monoclonal antibody (NAP40-2)-immobilized plate and a biotinylated IgG (biotinylated antibody No. 4998, biotinylated antibody No. 5036 or biotinylated antibody No. 5037).

FIG. 14 is a graph showing a cross-reactivity with rat Nesfatin-1 in the sandwitch ELISA using an anti-recombinant human Nesfatin-1 mouse monoclonal antibody (NAP40-2)-immobilized plate and a biotinylated antibody No. 4998.

FIG. 15 is a graph showing a cross-reactivity with Nesfatin (full molecule) in the sandwitch ELISA using an anti-recombinant human Nesfatin-1 mouse monoclonal antibody (NAP40-2)-immobilized plate and a biotinylated antibody No. 4998.

FIG. 16 is a graph showing a reactivity with human Nesfatin-1 in the sandwitch ELISA using an anti-Nesfatin-1 C-terminus peptide PAb (antibody No. 4998)-immobilized plate and a biotinylated IgG (biotinylated NAE1).

FIG. 17 is a graph showing a cross-reactivity with rat Nesfatin-1 in the sandwitch ELISA using an anti-Nesfatin-1 C-terminus peptide PAb (antibody No. 4998)-immobilized plate and a biotinylated IgG (biotinylated NAE1 or biotinylated NAE3).

FIG. 18 is a graph showing a cross-reactivity with NucB1-N77 in the sandwitch ELISA using an anti-Nesfatin-1 C-terminus peptide PAb (antibody No. 4998)-immobilized plate and a biotinylated IgG (biotinylated NAE1) prepared in the above-mentioned Example 14 (1) (A).

FIG. 19 is a graph showing a cross-reactivity with Nesfatin in the sandwitch ELISA using an anti-Nesfatin-1 C-terminus peptide PAb (antibody No. 4998)-immobilized plate and a biotinylated IgG (biotinylated NAE1) prepared in the above-mentioned Example 14 (1) (A).

FIG. 20 is a graph showing a reactivity with human Nesfatin in the sandwitch ELISA using an anti-Nesfatin IgG (antibody No. 4994)-immobilized plate and a biotinylated antibody (NAE1, NAE3 or NAD15).

FIG. 21 is a graph showing a cross-reactivity with rat Nesfatin in the sandwitch ELISA using a plate on which a mouse monoclonal antibody against human Nesfatin C-terminus peptide (antibody NAD15) was immobilized and a biotinylated antibody (NAE1 or NAE3).

FIG. 22 is a graph showing a cross-reactivity with human NucB1 in the sandwitch ELISA using an anti-Nesfatin IgG (antibody No. 4994)-immobilized plate and a biotinylated antibody (NAE1).

FIG. 23 is a graph showing a cross-reactivity with rat or mouse Nesfatin-1 in the sandwitch ELISA using an anti-Nesfatin-1 C-terminus peptide PAb (antibody No. 4998)-immobilized plate and a biotinylated IgG (NAF11).

FIG. 24 is a graph showing a cross-reactivity with mouse or rat Nesfatin in the sandwitch ELISA using an anti-Nesfatin-1 C-terminus peptide PAb (antibody No. 4994)-immobilized plate and a biotinylated antibody (antibody NAF11).

### BEST MODE FOR CARRYING OUT THE INVENTION

### Antibody Performing an Antigen-Antibody Reaction with Nesfatin-1, but not Substantially Performing the Antigen-Antibody Reaction with Nesfatin and/or NucB1>

The present invention relates to an antibody performing an antigen-antibody reaction with Nesfatin-1, but not substantially performing the antigen-antibody reaction with Nesfatin (Nucleobindin 2 (NucB2)) or NucB1; or an antibody performing an antigen-antibody reaction with Nesfatin-1, but not substantially performing the antigen-antibody reaction with Nesfatin and NucB1.

In the present invention, “Nesfatin-1” is a polypeptide having an activity suppressing food intake and/or body weight gain, represented by SEQ ID NOs: 5 to 7. Nesfatin-1 is considered to exhibit an activity suppressing food intake and/or body weight gain by cutting out from Nesfatin using a cleavage enzyme such as a prohormone convertase in vivo, and the like. Further, “Nesfatin-1” in the present invention also includes a polypeptide having an activity suppressing food intake and/or body weight gain, in which one to several amino acids are substituted/deleted/inserted in the amino acid sequence represented by SEQ ID NOs: 5 to 7. As such a polypeptide, specifically, a polypeptide in which a recognition site for a cleavage enzyme remains at the terminus when Nesfatin is digested or in which one to several amino acids are substituted/deleted/inserted when Nesfatin-1 is labeled, and having an activity suppressing food intake and/or body weight gain, and the like are mentioned.

Such Nesfatin-1 is obtained by cleaving a Nesfatin polypeptide having the amino acid sequence represented by any of SEQ ID NOs: 8 to 13 using a prohormone convertase, and then purifying using reversed phase chromatography and the like or conducting a process of binding and release to an antibody against a Nesfatin-1 polypeptide. Further, a recombinant Nesfatin-1 is obtained as described in Example 1.

One embodiment in the present invention is an antibody performing an antigen-antibody reaction with Nesfatin-1, but not substantially performing the antigen-antibody reaction with Nesfatin. Here, Nesfatin is referred to the polypeptide represented by SEQ ID NOs: 8 to 13. “Not substantially performing the antigen-antibody reaction with Nesfatin” means that when approximately the same number of molecules of each Nesfatin-1 and Nesfatin is immobilized on a solid phase and there each antibody is performed an antigen-antibody reaction, the binding level of the antibody on a solid phase immobilized Nesfatin is less than one-fifth of the binding level of the antibody on a solid phase immobilized Nesfatin-1. Such “an antibody performing an antigen-antibody reaction with Nesfatin-1, but not substantially performing the antigen-antibody reaction with Nesfatin” is obtained by using the peptide having the following amino acid sequence as an immunogen. Here, as an animal for immunizing the peptide, a rabbit is preferred.

NSF1-C18:-
(SEQ ID NO: 1)
Gly-Cys-Ser-Lys-Glu-Leu-Asp-Leu-Val-Ser-His-
His-Val-Arg-Thr-Lys-Leu-Asp-Glu-Leu

An antibody of the present invention may be a polyclonal antibody or a monoclonal antibody. It is shown in Example 5 that the polyclonal antibody is obtained reproducibly as an antibody performing an antigen-antibody reaction with Nesfatin-1, but not substantially performing the antigen-antibody reaction with Nesfatin by immunizing a rabbit with the above-mentioned NSF1-C18 peptide. As an example of the monoclonal antibody, a monoclonal antibody produced by a hybridoma (NAF7) deposited under the accession number: FERM ABP-10882 (Depositary Authority: International Patent Organism Depositary, National Institute of Advanced Industrial Science and Technology; Date of the acceptance: Jul. 27, 2007) is mentioned. In addition, the present invention also relates to such hybridoma.

Another embodiment of an antibody in the present invention is an antibody performing an antigen-antibody reaction with Nesfatin-1, but not substantially performing the antigen-antibody reaction with NucB1. Here, NucB1 is referred to the polypeptide represented by SEQ ID NOs: 14 to 16. In addition, in the present specification, a part having high homology with Nesfatin-1 particularly in primary structure is referred to as NucB1-N77 in the present invention, and shown by the structure represented by SEQ ID NOs: 17 to 19. Further, a recombinant NucB1-N77 has a structure in which “Gly-Ser” remains at the N-terminus. In addition, in the case of referring to “NucB1” in the present invention, “NucB1-N77” may also be included. “Not substantially performing the antigen-antibody reaction with NucB1” means that when approximately the same number of molecules of each Nesfatin-1 and NucB1 (NucB1-N77) is immobilized on a solid phase and there each antibody is performed an antigen-antibody reaction, the binding level of the antibody on a solid phase immobilized NucB1 is less than one-fifth of the binding level of the antibody on a solid phase immobilized Nesfatin-1.

An antibody of the present invention may be a polyclonal antibody or a monoclonal antibody. As an example of the monoclonal antibody, a monoclonal antibody produced by a hybridoma (NAE1, NAF7, NAF11 or NAP40-2) deposited under the accession number: FERM ABP-10881, FERM ABP-10882, FERM ABP-10883 or FERM ABP-10884 (Depositary Authority: International Patent Organism Depositary, National Institute of Advanced Industrial Science and Technology; Date of the acceptance: Jul. 27, 2007) is mentioned. In addition, the present invention also relates to such hybridoma.

As a further preferable embodiment of an antibody in the present invention, an antibody performing an antigen-antibody reaction with Nesfatin-1, but not substantially performing the antigen-antibody reaction with Nesfatin and NucB1 is mentioned. “Not substantially performing the antigen-antibody reaction with Nesfatin and NucB1” means satisfying both the requirement of “Not substantially performing the antigen-antibody reaction with Nesfatin” described above and the requirement of “Not substantially performing the antigen-antibody reaction with NucB1” described above.

An antibody of the present invention may be a polyclonal antibody or a monoclonal antibody. As described in the following Examples, a monoclonal antibody produced by a hybridoma (NAF7 or NAP40-2, respectively) deposited under the accession number: FERM ABP-10882 or FERM ABP-10884 (Depositary Authority: International Patent Organism Depositary, National Institute of Advanced Industrial Science and Technology; Date of the acceptance: Jul. 27, 2007) is particularly excellent in sensitivity of the antigen-antibody reaction with Nesfatin-1 and in the point of low cross-reactivity to Nesfatin and NucB1. In addition, the present invention also relates to such hybridoma.

Further, an antibody of the present invention is preferably not substantially performing an antigen-antibody reaction even with NucB1-N77. As such antibody, a monoclonal antibody produced by a hybridoma (NAF7) deposited under the accession number: FERM ABP-10882 (Depositary Authority: International Patent Organism Depositary, National Institute of Advanced Industrial Science and Technology; Date of the acceptance: Jul. 27, 2007) is mentioned.

In addition, as a method for obtaining the above-mentioned monoclonal antibody, a method of culturing hybridomas that produce desired antibodies, respectively and then purifying the antibodies from the obtained culture supernatant according to an ordinaly method is used. Further, as another method, a method of obtaining a gene encoding an antibody from a hybridoma producing the desired antibody, more specifically, a gene encoding heavy and light chains of immunoglobulin, producing a vector to express the gene, introducing the vector into a host cell (a mammalian cell, an insect cell, a microorganism and the like), and producing the antibody may be used. At that time, as for a gene encoding heavy and light chains of immunoglobulin, it is conducted to perform genetic modification to introduce the desired trait and to prepare an antibody-chimeric protein, a low-molecular-weight antibody and a scaffold antibody using variable regions of heavy and light chains of immunoglobulin by those skilled in the art using a known technique.

### <Immunological Detection Method for Nesfatin-1>

By using the above-mentioned “an antibody performing an antigen-antibody reaction with Nesfatin-1, but not substantially performing the antigen-antibody reaction with Nesfatin or NucB1; or an antibody performing an antigen-antibody reaction with Nesfatin-1, but not substantially performing the antigen-antibody reaction with Nesfatin and NucB1”, an immunological detection method of specifically detecting Nesfatin-1 is constructed. For example, the immunological detection method for Nesfatin-1 includes: a step of bringing a test substance into contact with an antibody performing an antigen-antibody reaction with Nesfatin-1, but not substantially performing the antigen-antibody reaction with Nesfatin and/or NucB1 (I), and a step of detecting an antibody performed an antigen-antibody reaction with the test substance (II). Further, the immunological detection method using the antigen-antibody reaction is well known in the art, and any methods conventionally used are employed in the present invention.

One embodiment of such immunological detection method is to measure the level of the antibodies performing an antigen-antibody reaction with Nesfatin-1. Further, one of the embodiments is a method using two antibodies among the above-mentioned antibodies. In this case, a preferable combination of antibodies to be used is a combination of an antibody performing an antigen-antibody reaction with Nesfatin-1, but not substantially performing the antigen-antibody reaction with Nesfatin and an antibody performing an antigen-antibody reaction with Nesfatin-1, but not substantially performing the antigen-antibody reaction with NucB1, a combination of an antibody performing an antigen-antibody reaction with Nesfatin-1, but not substantially performing the antigen-antibody reaction with Nesfatin and an antibody performing an antigen-antibody reaction with Nesfatin-1, but not substantially performing the antigen-antibody reaction with Nesfatin and NucB1, a combination of an antibody performing an antigen-antibody reaction with Nesfatin-1, but not substantially performing the antigen-antibody reaction with NucB1 and an antibody performing an antigen-antibody reaction with Nesfatin-1, but not substantially performing the antigen-antibody reaction with Nesfatin and NucB1, and a combination of two types of antibodies performing an antigen-antibody reaction with Nesfatin-1, but not substantially performing the antigen-antibody reaction with Nesfatin and NucB1. Here, in the combination of an antibody performing an antigen-antibody reaction with Nesfatin-1, but not substantially performing the antigen-antibody reaction with Nesfatin and an antibody performing an antigen-antibody reaction with Nesfatin-1, but not substantially performing the antigen-antibody reaction with NucB1, the former has the property not denying the antigen-antibody reaction with NucB1 and the latter has the property not denying the antigen-antibody reaction with Nesfatin. However, according to the combination, Nesfatin-1 is specifically detected, and Nesfatin and NucB1 are prevented from substantially being detected.

In an immunological measurement method using these two types of antibodies, there may be mentioned a method of immobilizing at least one type of antibody on a solid phase and a method of not immobilizing any of antibodies on a solid phase.

As a measurement method of immobilizing at least one type of antibody on a solid phase, a measurement method by a sandwich method is mentioned, which includes: a step of bringing a test substance into contact with the primary antibody immobilized on a solid phase (I), a step of bringing a labeled secondary antibody into contact with the test substance to which the primary antibody is bound (II), and a step of detecting the secondary antibody performed an antigen-antibody reaction with the test substance (III). The label for the secondary antibody in this measurement method is used for quantifying the level of antibody performing an antigen-antibody reaction with Nesfatin-1, and is generally performed with an enzyme, a radioisotope, a fluorescent substance or a luminescent substance. Here, when the secondary antibodies labeled with an enzyme are used, the level of the secondary antibodies performed an antigen-antibody reaction with Nesfatin-1 are measured by reacting a substance that develops color, luminescence and fluorescence according to a reaction with an enzyme with the secondary antibodies as a substrate of the enzyme, and by quantitatively measuring the resulting color (measured by absorbance), luminescence and fluorescence. In addition, at the same time, a standard substance containing the peptide is also subject to the measurement at the same time at a different concentration each time using a measurement value (background) in a control substance without containing Nesfatin-1 and a standard peptide of Nesfatin-1, and a standard curve showing a binding state in a dose-dependent manner is made. By using the result obtained from these, Nesfatin-1 contained in the test substance is determined. Further, the lowest concentration of Nesfatin-1, at which the detection is capable by this measurement method, defines the sensitivity of the measurement system. That is, the lowest concentration indicates the sensitivity as a concentration of Nesfatin-1 in a standard substance containing a standard peptide of Nesfatin-1 which shows the measurement value capable of significant discrimination, for the measurement value in a control substance without containing Nesfatin-1. In the present invention, even in a standard substance containing Nesfatin-1 at a low concentration of 30 pM, the discrimination from a control substance without containing Nesfatin-1 was conducted. Therefore, this measurement system is considered to have the detection sensitivity measurable even at less than 30 pM. In addition, according to a preferable embodiment of the present invention, even in a standard substance containing Nesfatin-1 at a concentration of less than 5 pM, the discrimination from a control substance without containing Nesfatin-1 is conducted.

As an actual example of such measurement system, as shown in Example 14, there may be mentioned a measurement system in which a primary antibody is a polyclonal antibody (antibody No. 4998) generated by immunizing NSF1-C18 into a rabbit and a secondary antibody is an antibody produced by a hybridoma (NAE1) deposited under the accession number: FERM ABP-10881. In such system, as for human Nesfatin-1, even at a concentration of a few number of pM, the discrimination from a control substance without containing Nesfatin-1 is conducted. It was shown to have the sensitivity of 30 pM or less, and further the sensitivity of around a few number of pM. Also, Nesfatin-1 contained in a test substance is detected with a high sensitivity even by using the antibody No. 4998 as a primary antibody, and an antibody produced by a hybridoma (NAF11) deposited under the accession number: FERM ABP-10883 as a secondary antibody.

In addition, Nesfatin-1 contained in a test substance is detected with a high sensitivity even by using an antibody produced by a hybridoma (NAP40-2) deposited under the accession number: FERM ABP-10884, performing an antigen-antibody reaction with Nesfatin-1 with a high sensitivity, but not substantially performing the antigen-antibody reaction with Nesfatin and NucB1 as a primary antibody, and using an antibody (antibody No. 4998) of the present invention performing an antigen-antibody reaction with Nesfatin-1, but not substantially performing the antigen-antibody reaction with Nesfatin as a secondary antibody. Also, human Nesfatin-1 contained in a test substance is detected with a high sensitivity even by using an antibody produced by a hybridoma (NAF7) deposited under the accession number: FERM ABP-10882 as a primary antibody, and the antibody No. 4998, No. 6151 or No. 6152 as a secondary antibody.

As a method of not immobilizing any of the two types of antibodies on a solid phase, FRET (Fluorescence Resonance Energy Transfer) and BRET (Bioluminescence Resonance Energy Transfer) are mentioned. These analyses are performed by using two types of antibodies performing an antigen-antibody reaction with Nesfatin-1, in which one antibody is bound to a fluorescent substance or a luminescent protein, and the other antibody is labeled with a fluorescent substance absorbing the fluorescent or luminescent light energy at a specific wavelength. This energy transfer is required that a fluorescent substance or a luminescent protein emitting light and a fluorescent substance absorbing the light energy present in the immediate vicinity of each other. Therefore, only when the two types of antibodies are performed an antigen-antibody reaction with one antigen, the state of the antibody performing an antigen-antibody reaction with Nesfatin-1 that is an antigen is quantitatively detected by detecting the quenching of the light emitting from a fluorescent substance or a luminescent protein of the primary antibody and the fluorescence at a wavelength of the light emitting from a fluorescent substance bound to the secondary antibody.

Further, as a manner of another measurement method, a measurement method by a competitive antigen-antibody reaction may also be mentioned, in which a test substance is mixed with the labeled standard peptide of Nesfatin-1 adjusted the concentration thereof in advance, and competitively reacted with an antibody performing an antigen-antibody reaction with Nesfatin-1. In this case, a label for Nesfatin-1 is used for quantifying the standard peptide bound to an antibody, and is generally performed with a radioisotope, a fluorescent substance, a luminescent substance and an enzyme are employed. Here, when the standard peptide labeled with an enzyme is used, the level of the standard peptide performed an antigen-antibody reaction with an antibody is measured by reacting a substance that develops color, luminescence and fluorescence according to a reaction with an enzyme with the standard peptide as a substrate of the enzyme, and by quantitatively measuring the resulting color (measured by absorbance), luminescence and fluorescence. Here, as an antibody to be used, any antibody may be used as long as it performs an antigen-antibody reaction with both the labeled standard peptide of Nesfatin-1 and the Nesfatin-1 contained in a test substance, preferably an antibody performing an antigen-antibody reaction with Nesfatin-1, but not substantially performing the antigen-antibody reaction with Nesfatin and NucB1 is used, and more preferably a monoclonal antibody produced by a hybridoma (NAF7 or NAP40-2) deposited under the accession number: FERM ABP-10882 or FERM ABP-10884 is desirably used. In addition, at the same time, a standard substance containing the peptide is also subject to the measurement at the same time at a different concentration each time using a measurement value (background) in a control substance without containing Nesfatin-1 and a standard peptide of Nesfatin-1 without labeling, and a standard curve showing a binding state in a dose-dependent manner is made. By using the result obtained from these, Nesfatin-1 contained in a test substance is determined.

A test substance applied for an immunological detection method of the present invention may be any test substance expecting the detection of Nesfatin-1, for example, blood, serum, spinal fluid, urine, ascitic fluid, pleural fluid, saliva, lacrimal fluid and expectoration obtained from a patient, a tissue obtained by biopsy and the like are mentioned.

### <Detection Kit Used for an Immunological Detection Method of Nesfatin-1>

The present invention also relates to a kit used for an immunological detection method of Nesfatin-1 as described above. For example, a kit in the above-mentioned measurement method by a sandwich method includes a solid phase on which the above-mentioned primary antibody described above is immobilized, and the labeled secondary antibody described above. A reagent used for labeling the secondary antibody is known in the art, for example, biotin, a fluorescent substance, a luminescent substance, and an enzyme (peroxidase, phosphatase, glucosidase, luciferase and the like) are mentioned. Here, when the secondary antibody labeled with an enzyme is used, the kit may also include a substrate of an enzyme that develops color, luminescence and fluorescence according to a reaction with an enzyme.

A kit of the present invention used for the immunological detection method of Nesfatin-1 by a sandwich method of the present invention may further include a standard peptide. A standard peptide included in a kit of the present invention is used for making a standard curve showing a binding avidity of the above-mentioned antibody with Nesfatin-1 in a dose-dependent manner. As such a standard polypeptide, Nesfatin-1 may be used.

A kit of the present invention including these is used for detecting the level of Nesfatin-1 contained in a test substance by, for example, making a standard curve by adding a standard peptide to a primary antibody immobilized on a solid phase to react, and then further adding a labeled secondary antibody (1); similarly adding a test substance to an immobilized primary antibody to react, and then adding the labeled secondary antibody (2); and measuring the binding level of the secondary antibody in the reaction with the test substance by using the standard curve (3).

As an embodiment of another kit, a kit used for a measurement method such as FRET, BRET and the like as described above is mentioned, and the kit includes a primary antibody labeled with a fluorescent substance or a luminescent protein and a secondary antibody labeled with a fluorescent substance absorbing the light energy emitted at a specific wavelength from the fluorescent substance or luminescent substance. In addition, a kit used for an immunological detection method of Nesfatin-1 by FRET or BRET of the present invention may further include a standard peptide to make a standard curve showing a binding avidity of the above-mentioned antibody with Nesfatin-1 in a dose-dependent manner. As such a standard polypeptide, Nesfatin-1 may be used.

Further, as an embodiment of another kit, a kit used for a measurement method by a competitive antigen-antibody reaction is mentioned, the kit includes a labeled Nesfatin-1 standard peptide and an antibody performing an antigen-antibody reaction with Nesfatin-1. A reagent used for labeling a standard peptide is known in the art, for example, biotin, a fluorescent substance, a luminescent substance, and an enzyme (peroxidase, phosphatase, glucosidase, luciferase and the like) are mentioned. Here, when the standard peptide labeled with an enzyme is used, the kit may also include a substrate of an enzyme that develops color, luminescence and fluorescence according to a reaction with an enzyme. In addition, a kit used for an immunological detection method of Nesfatin-1 by a competitive antigen-antibody reaction of the present invention may further include a standard peptide to make a standard curve showing a binding avidity of the above-mentioned antibody with Nesfatin-1 in a dose-dependent manner. As such a standard polypeptide, Nesfatin-1 may be used.

In addition, a kit of the present invention may include a buffer solution to dilute a reagent or a biological sample, a substrate to measure a positive control, a negative control and a label, a reaction vessel, an instruction described an assay protocol and the like as an additional component. These components may be mixed in advance as necessary. In addition, a preservative and an antiseptic agent may be contained in each component as necessary.

<Antibody Performing an Antigen-Antibody Reaction with Nesfatin, but not Substantially Performing the Antigen-Antibody Reaction with NucB1>

The present invention relates to an antibody performing an antigen-antibody reaction with Nesfatin, but not substantially performing the antigen-antibody reaction with NucB1. “Not substantially performing the antigen-antibody reaction with NucB1” means that when approximately the same number of molecules of each Nesfatin and NucB1 is immobilized on a solid phase and there each antibody is performed an antigen-antibody reaction, the binding level of the antibody on a solid phase immobilized NucB1 is less than one-fifth of the binding level of the antibody on a solid phase immobilized Nesfatin.

“An antibody performing an antigen-antibody reaction with Nesfatin, but not substantially performing the antigen-antibody reaction with NucB1” of the present invention is especially referred to an specific antibody performing an antigen-antibody reaction with Nesfatin with a high sensitivity, but not substantially performing the antigen-antibody reaction with NucB1. That is, an antibody of the present invention may be a polyclonal antibody and may also be a monoclonal antibody. Among these, a monoclonal antibody produced by a hybridoma (NAE1 or NAF11) deposited under the accession number: FERM ABP-10881 or FERM ABP-10883 is particularly preferred. In addition, the present invention also relates to such hybridoma.

Further, as another embodiment, an antibody performing an antigen-antibody reaction with Nesfatin, but not substantially performing the antigen-antibody reaction with Nesfatin-1 and NucB1 is mentioned. “Not substantially performing the antigen-antibody reaction with Nesfatin-1 and NucB1” means that when approximately the same number of molecules of each Nesfatin and Nesfatin-1 is immobilized on a solid phase and there each antibody is performed an antigen-antibody reaction, the binding level of the antibody on a solid phase immobilized Nesfatin-1 is less than one-fifth of the binding level of the antibody on a solid phase immobilized Nesfatin, in addition to the requirement “not substantially performing the antigen-antibody reaction with NucB1” as described above. An antibody of the present invention may be a polyclonal antibody and also may be a monoclonal antibody.

### <Immunological Detection Method of Nesfatin>

By using “an antibody performing an antigen-antibody reaction with Nesfatin, but not substantially performing the antigen-antibody reaction with NucB1” or “an antibody performing an antigen-antibody reaction with Nesfatin, but not substantially performing the antigen-antibody reaction with Nesfatin-1 and NucB1” described above, an immunological detection method of specifically detecting Nesfatin is constructed. For example, the immunological detection method for Nesfatin includes: a step of bringing a test substance into contact with the antibody performing an antigen-antibody reaction with Nesfatin, but not substantially performing the antigen-antibody reaction with NucB1 (I), and a step of detecting an antibody performing an antigen-antibody reaction with the test substance (II). Further, the immunological detection method using an antigen-antibody reaction is well known in the art, and any methods conventionally used are employed in the present invention.

One embodiment of such immunological detection method is to measure the level of the antibodies performing an antigen-antibody reaction with Nesfatin. Further, one of the embodiments is a method using two antibodies among the above-mentioned antibodies. In this case, a preferable combination of antibodies to be used is a combination using two antibodies of which at least one is an antibody performing an antigen-antibody reaction with Nesfatin, but not substantially performing the antigen-antibody reaction with NucB1.

In an immunological measurement method using these two types of antibodies, there may be mentioned a method of immobilizing at least one type of antibody on a solid phase and a method of not immobilizing any of antibodies on a solid phase.

As a measurement method of immobilizing at least one type of antibody on a solid phase, a measurement method by a sandwich method is mentioned, which includes: a step of bringing a test substance into contact with a primary antibody immobilized on a solid phase (I), a step of bringing a labeled secondary antibody into contact with the test substance to which the primary antibody is bound (II), and a step of detecting the secondary antibody performing an antigen-antibody reaction with the test substance (III). Labeling, a measurement technique and the like of a secondary antibody in an measurement method by such sandwich method are performed similarly to the measurement method by such sandwich method in <Immunological detection method for Nesfatin-1> as described above. Further, the lowest concentration of Nesfatin, at which the detection is capable by this measurement method, defines the sensitivity of the measurement system. That is, the lowest concentration indicates the sensitivity as a concentration of Nesfatin in a standard substance containing a standard peptide of Nesfatin which shows the measurement value capable of significant discrimination, for the measurement value in a control substance without containing Nesfatin. In the present invention, even in a standard substance containing Nesfatin at a low concentration of 30 pM, the discrimination from a control substance without containing Nesfatin was conducted. Therefore, this measurement system is considered to have the detection sensitivity measurable even at less than 30 pM. In addition, according to a preferable embodiment of the present invention, even in a standard substance containing Nesfatin at a concentration of less than 5 pM, the discrimination from a control substance without containing Nesfatin-1 is conducted.

Among these, Nesfatin contained in a test substance is detected with a high sensitivity by using an antibody (antibody No. 4994 or NAD15) of the present invention performing an antigen-antibody reaction with Nesfatin, but not substantially performing the antigen-antibody reaction with NucB1 as the primary antibody, and using an antibody produced by a hybridoma (NAE1 or NAF11) deposited under the accession number: FERM ABP-10881 or FERM ABP-10883 and detecting Nesfatin with a high sensitivity as the secondary antibody. Further, Nesfatin is detected with a high sensitivity by using an antibody produced by a hybridoma (NAD15) and performing an antigen-antibody reaction with Nesfatin, but not substantially performing the antigen-antibody reaction with Nesfatin-1 and NucB1 as the primary antibody.

As a method of not immobilizing both two types of antibodies on a solid phase, FRET (Fluorescence Resonance Energy Transfer) and BRET (Bioluminescence Resonance Energy Transfer) are mentioned.

Further, as a manner of another measurement method, a measurement method by a competitive antigen-antibody reaction may also be mentioned, in which a test substance is mixed with a labeled standard peptide of Nesfatin adjusted the concentration thereof in advance, and the resultant is competitively reacted to conduct a binding to an antibody performing an antigen-antibody with Nesfatin. The measurement method by a competitive antigen-antibody reaction is also performed similarly to the measurement method by a competitive antigen-antibody reaction in <Immunological detection method for Nesfatin-1> as described above.

A test substance applied for an immunological detection method of the present invention may be any test substance expecting the detection of Nesfatin, for example, blood, serum, spinal fluid, urine, ascitic fluid, pleural fluid, saliva, lacrimal fluid and expectoration obtained from a patient, a tissue obtained by biopsy and the like are mentioned.

### <Kit Used for an Immunological Detection Method of Nesfatin>

The preset invention also relates to a kit used for an immunological detection method of Nesfatin described above. For example, a kit in the above-mentioned measurement method by a sandwich method includes a solid phase on which the above-mentioned primary antibody is immobilized, and the labeled secondary antibody described above. Further, a kit used for an immunological detection method of Nesfatin of the present invention may further include a standard peptide, a substrate of an enzyme and the like which is similar to the standard peptide and the like as described in <Kit used for an immunological detection method of Nesfatin-1>, and may be used similarly.

As an embodiment of another kit, a kit used for a measurement method such as FRET, BRET and the like as described above includes a primary antibody labeled with a fluorescent substance or a luminescent protein and a secondary antibody labeled with a fluorescent substance absorbing the light energy emitted at a specific wavelength from the fluorescent substance or luminescent substance. In addition, a kit used for an immunological detection method of Nesfatin by FRET or BRET of the present invention may further include a standard peptide and the like which is similar to the standard peptide and the like as described in <Kit used for an immunological detection method of Nesfatin-1>.

Further, as an embodiment of another kit, a kit used for a measurement method by a competitive antigen-antibody reaction is mentioned, the kit includes a labeled Nesfatin standard peptide and an antibody performing an antigen-antibody reaction with Nesfatin. Further, a kit used for an immunological detection method of Nesfatin by a competitive antigen-antibody reaction of the present invention may further include a standard peptide, a substrate of an enzyme and the like.

<Pharmaceutical Composition for Food Intake Facilitation and/or Weight Gain Facilitation>

The above-mentioned antibody of the present invention is used for a pharmaceutical composition together with a pharmaceutically acceptable carrier and/or diluent. The pharmaceutical composition is used for a disease or symptom where the suppression of food intake and the suppression of weight gain becomes a problem. As a disease or symptom where the suppression of food intake and the suppression of weight gain becomes a problem, for example, anorexia, functional dyspepsia, or a state of food intake suppression and/or weight gain suppression according to a cancer, an inflammatory disease, a decline in the function of pituitary gland, thyroid gland, adrenal gland and the like, after surgery or an extreme stress, and others. This pharmaceutical composition is formed into various formulations and administered orally or parenterally. As a parenteral administration, for example, an intravenous administration, a subcutaneous administration, an intramuscular administration, a percutaneous administration or an intrarectal administration is mentioned.

A formulation containing an antibody of the present invention as an active component is prepared by using a carrier and an excipient that are usually used for formulation, and other additives. A carrier and an excipient for formulation may be in any state of solid or liquid, for example, lactose, magnesium stearate, starch, talc, gelatin, agar, pectin, gum Arabic, olive oil, sesame oil, cacao butter, ethylene glycol and the like, and other components used ordinary are mentioned. Administration may be performed in any form of oral administration by tablets, pills, capsules, granules, powders, liquid and the like or parenteral administration by injection for intravenous injection, intramuscular injection and the like, suppository, percutaneous administration and others.

An antibody of the present invention varies depending on a type of disease, a route of administration, a symptom of patient, age, sex, weight and the like, but usually is administered in the range of 0.1 to 500 mg, preferably 0.5 to 20 mg at one time per adult. However, since the dose varies depending on various conditions, the dose may be sufficient at lower than the dose described above, or may be required higher doses than the above-mentioned range.

Hereinafter, the present invention is further explained in detail with reference to Examples, but the scope of the present invention is not restricted to these Examples.

### EXAMPLES

### Example 1

### Preparation of Recombinant NESFATIN-1 by Recombinant Technology

In order to prepare NESFATIN-1 in larger scale, a recombinant NESFATIN-1 was prepared by using recombinant technology. Specifically, a gene encoding human NESFATIN-1 was obtained, and at the N-terminus a gene of GST (glutathione-S-transferase) and a histidine tag was bound, and then an expression vector was constructed so that a cleavage site (-Leu-Val-Pro-Arg-Gly-Ser-) by thrombin may intervene between an amino acid sequence of a histidine tag and an amino acid sequence of human NESFATIN-1 in the protein after translation. The details are as follows.

A gene of the human Nesfatin-1 was obtained by performing PCR (Nested PCR) twice using as a template cDNA obtained by synthesizing from human Hypothalamus mRNA (Clontech Corporation) using Super Script III (Invitrogen Corporation).

The reaction of PCR in the first round was performed by using the following forward (hNucB2-F0191: SEQ ID NO: 20) and reverse (hNucB2-R1549: SEQ ID NO: 21) at each concentration of 100 pM, Pyrobest DNA polymerase (R005A, TAKARA BIO INC.), the attached reaction buffer and dNTP, in accordance with the attached protocol. The PCR reaction was performed 30 cycles of the temperature cycle at 98° C. for 10 seconds and 68° C. for 1 minute and 30 seconds after the incubation at 90° C. for 1 minute, and then under the temperature condition of 68° C. for 2 minutes.

Forward Primer (hNucB2-F0191):
(SEQ ID NO: 20)
5′-GGAGATAAAAATTATTTACCTGCCTGAACA-3′
Reverse Primer (hNucB2-R1549):
(SEQ ID NO: 21)
5′-AAATATTTATTGAGCAGAGAAAAGGGAAGG-3′

By using 0.5 μL of the obtained PCR product as a template, the reaction of PCR in the second round was performed. By using the following forward (hNucB2-F292[Sac2-Thr]) and reverse (hNucB2-R514[NotI]) primers at a concentration of 100 pM, the reaction of PCR was performed similarly to the PCR reaction in the first round using Pyrobest DNA polymerase. The PCR reaction was performed 20 cycles of the temperature cycle at 98° C. for 10 seconds, 60° C. for 30 seconds, and 68° C. for 1 minute after the incubation at 90° C. for 1 minute, and then under the temperature condition of 68° C. for 2 minutes.

Forward Primer (hNucB2-F292[Sac2-Thr]):
(SEQ ID NO: 22)
5′-GGTTCCGCGGGTCTGGTTCCGCGTGGTTCTGTGCCTATTGATAT
AGACAAGACAAAAGT-3′
Reverse Primer (hNucB2-R514[NotI]):
(SEQ ID NO: 23)
5′-GGTTGCGGCCGCTTACAGTTCATCAAGTTTTGTCCTCAC-3′

PCR reaction sample after performing the second-round PCR was purified by phenol/chloroform extraction, and then cleaved by restriction enzymes SacII and NotI. The fragments were subjected to agarose gel electrophoresis, and then a band corresponding to the length of around 300 by was cut out, and purified by using a QIAEX-II kit (QIAGEN Inc.). The purified PCR product with around 300 by was subjected to ligation using a Quick DNA ligase kit (New England Biolabs, Inc.) to a pET41a (+) plasmid vector (Novagen) cleaved by restriction enzymes SacII and NotI. The ligated vector was introduced into an Escherichia coli strain JM109, a small-scale plasmid extraction was performed with the obtained two transformants, and a base sequence analysis of the NESFATIN-1 gene sequence incorporated using the obtained plasmid was performed by using an autosequencer CEQ8000 of Beckman Coulter with the use of a CEQ DTCS Quick Start Kit. As a result, it was confirmed that an expression vector in which a gene having a correct NESFATIN-1 sequence was incorporated was obtained. This was named “pET41a(+)GST-His-LVPRGS-hNSF1”.

The obtained pET41a(+)GST-His-LVPRGS-hNSF1 was introduced into an Escherichia coli BL21 (DE3) Codon Plus RIPL and expressed, and as a result, a fusion protein (GST-His-LVPRGS-hNSF1) of GST/histidine tag/thrombin cleavage sequence/NESFATIN-1 was expressed. pET41a(+)GST-His-LVPRGS-hNSF1 was introduced into an Escherichia coli BL21 (DE3) Codon Plus RIPL, and then a clone obtained by the selection in Luria-Bertani (LB) broth containing kanamycin was cultured in LB broth containing 10 mL of kanamycin at 37° C. The cultivation was terminated at the time when the absorbance at a wavelength of 600 nm reached around 1.0 in the culture solution. A 3-mL aliquot of the culture solution was subcultured to the LB broth containing 100 mL of kanamycin, and the resultant broth was further cultured at 37° C., and at the time when the absorbance at a wavelength of 600 nm reached 0.8 in the culture solution, 1 mL of 100 mM isopropyl thiogalactoside (IPTG) was added to induce protein expression. After adding IPTG, the resultant broth was further cultured for 3 hours at 37° C. while shaking. The resultant culture solution was centrifuged at 8000 rpm for 20 minutes (at 4° C.) to recover the biomass the Escherichia coli.

The obtained Escherichia coli biomass was fractured by sonication and centrifuged, and the lysate containing the fusion protein (GST-His-LVPRGS-hNSF1) was extracted and purified using a nickel chelate column (Ni-NTA agarose). The biomass was suspended in Sonication Buffer (50 mM KH₂PO₄, 50 mM NaCl, 2 mM DTT, pH7.5) containing 20 mL of one-fold concentration of Complete-EDTA free (Roche Diagnostics K.K.) and 0.5-fold concentration of Bug Buster (Merck, Novagen Cat. No. 70584), and fractured by sonication in ice water for 10 minutes. Sample after the sonication treatment was centrifuged at 15,000 rpm for 20 minutes to recover the supernatant. A 10-mL aliquot of the obtained supernatant was applied to 1 mL of Ni-NTA agarose column equilibrated with Lysis Buffer (50 mM NaH₂PO₄, 300 mM NaCl, 10 mM imidazole, pH 8.0), and then washed twice with 10 mL of Wash Buffer (50 mM NaH₂PO₄, 300 mM NaCl, 20 mM imidazole, pH 8.0). The column after washing was eluted twice with 2.5 mL of Elution Buffer (50 mM NaH₂PO₄, 300 mM NaCl, 250 mM imidazole, pH 8.0), and a fraction containing the eluted fusion protein (GST-His-LVPRGS-hNSF1) was recovered. The extracted supernatant from the remaining biomass was treated similarly, and a fraction containing the fusion protein (GST-His-LVPRGS-hNSF1) was recovered.

A portion of the GST and histidine tag was removed from the fusion protein (GST-His-LVPRGS-hNSF1) and the remaining portion was further purified, and furthermore, in order to remove Escherichia coli-derived lipopolysaccharide (LPS) acting as an inflammatory substance, thrombin treatment, and purification by reversed-phase chromatography were performed in the state that the fusion protein (GST-His-LVPRGS-hNSF1) was bound to a GST resin. Further, in the subsequent treatment, the buffer confirmed that LPS was not contained in there was used. A 7.2-mL aliquot of the fraction containing the fusion protein (GST-His-LVPRGS-hNSF1) obtained by the purification in the Ni-NTA agarose column was washed with one-fold concentration of GST Bind/Wash Buffer (Merck, Novagen Cat. No. 70571), and finally the fraction was added to the GST resin (Novagen Cat. No. 70541, Merck) (equivalent to 7.2 mL) suspended with 3 mL of GST Bind/Wash Buffer, and the resultant mixture was gently stirred at 20° C. for 1 hour. The resin was recovered by centrifugation, and then washed twice with 36 mL of GST Bind/Wash Buffer. A 3.6-mL aliquot of a solution in which 20 units/mL of thrombin was dissolved in PBS (Phosphate Buffered Saline) was added and suspended in the washed resin, and the resultant was reacted for 20 hours while gently stirring at 20° C. The resin after the reaction was dispensed by 1.8 mL in cups (millipore) with a filter having a pore size of 0.22 μm, centrifuged at 3,000 rpm for 2 minutes, and then the filtered samples after thrombin treatment were recovered. To 450 μL of the obtained sample after thrombin treatment, 50 μL of acetic acid was added to prepare a sample for C18 reversed-phase chromatography. The reversed-phase chromatography analysis was performed by using a gradient elution method of acetonitrile in the presence of 0.1% trifluoroacetic acid, and setting the gradient as follows: 10% acetonitrile for 10 minutes, 10 to 20% acetonitrile gradient in 60 minutes, 30 to 50% acetonitrile gradient in 80 minutes, and 50 to 60% acetonitrile gradient in 5 minutes. The protein eluted from the column was monitored by measuring the absorbance at the wavelength of 230 nm or 280 nm. When the fraction eluted by the gradient of acetonitrile was analyzed by an SDS-PAGE analysis and a Western blotting analysis, it was found that NESFATIN-1 was eluted at the acetonitrile concentration of around 40%. Therefore, the fraction was recovered and freeze-dried, and then the resultant sample was dissolved again in distilled water for injection. By using the resultant solution, the protein level and the content of LPS were measured by absorbance and by endospacy (SEIKAGAKU CORPORATION), respectively.

The recombinant NESFATIN-1s of mouse and rat were also prepared similarly. The used templates and primers are as follows.

TABLE 1

| | mouse | rat |
|---|---|---|
| templates | mouse Brain cDNA | cDNA obtained by |
| | (Clontech Corporation) | synthesizing from rat |
| | | pancreas total RNA (Clontech |
| | | Corporation) using Super |
| | | Script III (Invitrogen |
| | | Corporation) |
| 1st PCR Forward Primer | mNucB2-F337: SEQ ID NO: 30 | rNucB2-F204: SEQ ID NO: 41 |
| 1st PCR Reverse Primer | mNucB2-R1613: SEQ ID NO: 31 | rNucB2-R1540: SEQ ID NO: 42 |
| 2nd PCR Forward Primer | mNucB2-F360: SEQ ID NO: 32 | ratNucB2-F286: SEQ ID NO: 43 |
| 2nd PCR Reverse Primer | mNucB2-R582: SEQ ID NO: 33 | ratNucB2-R507: SEQ ID NO: 44 |

### Example 2

### Preparation of Recombinant NESFATIN-Related Protein by Recombinant Technology

In order to prepare NESFATIN and NESFATIN-related proteins (NESFATIN-N27K and NESFATIN-C21K) in large scale, recombinant NESFATIN and NESFATIN-related proteins were prepared by using recombinant technology. Specifically, a gene encoding human NESFATIN was obtained, and at the N-terminus a gene of GST (glutathione-S-transferase) and a histidine tag was bound, and then an expression vector was constructed so that a cleavage site (-Leu-Val-Pro-Arg-Gly-Ser-) by thrombin may intervene between an amino acid sequence of a histidine tag and an amino acid sequence of human NESFATIN in a protein after translation. The details are as follows.

By using 0.5 μL of the PCR product obtained by the first round PCR in Example 1 as a template, the second round PCR was performed under the same conditions and using the same reagent as in the second round PCR in Example 1 except for using the following forward (hNucB2-F292 [Sac2-Thr]) and reverse (hNucB2-R1461[NotI]) primers.

Forward Primer (hNucB2-F292 [Sac2-Thr]):
(SEQ ID NO: 22)
5′-GGTTCCGCGGGTCTGGTTCCGCGTGGTTCTGTGCCTATTGATAT
AGACAAGACAAAAGT-3′
Reverse Primer (hNucB2-R1461[NotI]):
(SEQ ID NO: 24)
5′-GGTTGCGGCCGCGACTTTAAATGTGTGGCTCAAACTTC-3′

PCR reaction sample after performing the second round PCR was purified by phenol/chloroform extraction, and then cleaved by restriction enzymes SacII and NotI. The fragments were subjected to agarose gel electrophoresis, and then a band corresponding to the length of around 1.2K by was cut out, and purified by using a QIAEX-II kit (QIAGEN Inc.). The purified PCR product with around 1.2K by was subjected to ligation using a Quick DNA ligase kit (New England Biolabs, Inc.) to a pET41a (+) plasmid vector (Novagen) cleaved by restriction enzymes SacII and NotI. The ligated vector was introduced into an Escherichia coli strain JM109, a small-scale plasmid extraction was performed with the obtained two transformants. By using the obtained plasmid, a base sequence analysis of the incorporated NESFATIN-gene sequence was performed as in Example 1. As a result, it was confirmed that an expression vector in which a gene having a correct human NESFATIN sequence was incorporated was obtained. This was named “pET41a(+)GST-His-LVPRGS-hNSF full”.

By using the obtained pET41a(+)GST-His-LVPRGS-hNSF full, as in Example 1, a fusion protein (GST-His-LVPRGS-hNSF full) of GST/histidine tag/thrombin cleavage sequence/Nesfatin was expressed and recovered. Further, as in Example 1, NESFATIN was obtained by removing a portion of the GST and histidine tag from the fusion protein (GST-His-LVPRGS-hNSF full), treating with thrombin and purifying using reversed-phase chromatography. The gradient of acetonitrile was used setting the same as in Example 1. Further, there was also one site of a thrombin cleavage sequence in NESFATIN, NESFATIN may be split by thrombin treatment. Therefore, as a result of the thrombin treatment, the generated side of N-terminus of NESFATIN was referred to NESFATIN-N27K and the generated side of C-terminus of NESFATIN was referred to NESFATIN-C21K. When the fraction eluted by the gradient of acetonitrile was analyzed by an SDS-PAGE analysis and a Western blotting analysis, it was found that NESFATIN was eluted at the acetonitrile concentration of around 40%. Further, it was found that NESFATIN-N27K and NESFATIN-C21K had the peaks after and before NESFATIN, respectively. Therefore, the fraction was recovered and NESFATIN, NESFATIN-N27K and NESFATIN-C21K were obtained.

The recombinant NESFATINs and NESFATIN-related proteins of mouse and rat were also prepared similarly. However, A gene of the mouse and rat NESFATINs and NESFATIN-related proteins were obtained by performing PCR triple, the third round PCR was performed under the same conditions as second round PCR. The used templates and primers are as follows.

TABLE 2

| | mouse | rat |
|---|---|---|
| templates | mouse Brain cDNA | cDNA obtained by synthesizing |
| | (Clontech Corporation) | from rat pancreas total RNA |
| | | (Clontech Corporation) using |
| | | Super Script III (Invitrogen |
| | | Corporation). |
| 1st PCR Forward Primer | mNucB2-F337: SEQ ID NO: 30 | rNucB2-F204: SEQ ID NO: 41 |
| 1st PCR Reverse Primer | mNucB2-R1613: SEQ ID NO: 31 | rNucB2-R1540: SEQ ID NO: 42 |
| 2nd PCR Forward Primer | mNucB2-F360: SEQ ID NO: 32 | ratNucB2-F286: SEQ ID NO: 43 |
| 2nd PCR Reverse Primer | mNucB2-R1527: SEQ ID NO: 34 | ratNucB2-R1531: SEQ ID NO: 45 |
| 3rd PCR Forward Primer | His-Thr-For: SEQ ID NO: 35 | His-Thr-For: SEQ ID NO: 35 |
| 3rd PCR Reverse Primer | mNucB2-R1527: SEQ ID NO: 34 | ratNucB2-R1531: SEQ ID NO: 45 |

### Example 3

### Preparation of Recombinant NucB1-N77 by Recombinant Technology

In order to prepare in large scale the portion (NucB1-N77) corresponding to Nesfatin-1 of NucB1 having high homology with NucB2, recombinant human NucB1-N77 was prepared using recombinant technology. The recombinant NucB1-N77s of mouse and rat were also prepared similarly. Specifically, a gene encoding human NucB1-N77 was obtained, and at the N-terminus a gene of GST (glutathione-S-transferase) and a histidine tag was bound, and then an expression vector was constructed so that a cleavage site (-Leu-Val-Pro-Arg-Gly-Ser-) by thrombin may intervene between an amino acid sequence of a histidine tag and an amino acid sequence of the human NucB1-N77 in a protein after translation. The details are as follows.

A gene of the human NucB1-N77 was obtained by performing PCR (Nested PCR) twice using as a template cDNA synthesized from human Hypothalamus mRNA (Clontech Laboratories) using Super Script III (Invitrogen Corporation).

The first round PCR was performed under the same conditions and using the same reagent as in Example 1 except for using the following forward (hNucB1-F061: SEQ ID NO: 25) and reverse (hNucB1-R1376 [NotI]: SEQ ID NO: 26) primers.

Forward Primer (hNucB1-F061):
(SEQ ID NO: 25)
5′-TGCTGCTGCTGCTCCTGCTT-3′
Reverse Primer (hNucB1-R1376[NotI]):
(SEQ ID NO: 26)
5′-GGTTGCGGCCGCTCACAGATGCTGGGGCACCTCAACCTCA-3′

By using 0.5 μL of the obtained PCR product as a template, the second round PCR was performed under the same conditions and using the same reagent as in the second round of PCR in Example 1 except for using the following forward (hNucB1-F096 [Sac2Thr]) and reverse (hNucB1-R303[NotI]) primers.

Forward Primer (hNucBl-F096[Sac2Thr]):
(SEQ ID NO: 27)
5′-GGTTCCGCGGGTCTGGTTCCGCGTGGTTCTGTCCCCCTGGAGCG
AGGGGCGCCCAAC-3′
Reverse Primer (hNucB1-R303[NotI]):
(SEQ ID NO: 28)
5′-GGTTGCGGCCGCTCAGAGCTCATCCAGCTTGGTGCGGAC-3′

PCR reaction sample after performing the second-round PCR was purified by phenol/chloroform extraction, and then cleaved by restriction enzymes SacII and NotI. The fragments were subjected to agarose gel electrophoresis, and then a band corresponding to the length of around 300 by was cut out, and purified using a QIAEX-II kit (QIAGEN Inc.). The purified PCR product with around 300 by was subjected to ligation using a Quick DNA ligase kit (New England Biolabs, Inc.) to a pET41a (+) plasmid vector (Novagen) cleaved by restriction enzymes SacII and NotI. The ligated vector was introduced into an Escherichia coli strain JM109, a small-scale plasmid extraction was performed with the obtained two transformants. By using the obtained plasmid, a base sequence analysis of the incorporated human NucB1-N77 gene sequence was performed as in Example 1. As a result, it was confirmed that an expression vector in which a gene having a correct human NucB1-N77 sequence was incorporated was obtained. This was named “pET41a(+)GST-His-LVPRGS-hNucB1-N77”.

By using the obtained pET41a(+)GST-His-LVPRGS-hNucB1-N77, as in Example 1, a fusion protein (GST-His-LVPRGS-hNucB1-N77) of GST/histidine tag/thrombin cleavage sequence/human NucB1-N77 was expressed and recovered. Further, as in Example 1, removal of the portion of the GST and histidine tag from the fusion protein (GST-His-LVPRGS-hNucB1-N77), thrombin treatment, and purification by reversed-phase chromatography were performed. Further, the reversed-phase chromatography was performed by using a gradient elution method of acetonitrile in the presence of 0.1% trifluoroacetic acid as in Example 1. In addition, the gradient of acetonitrile was performed by setting the gradient as follows: 10% acetonitrile for 10 minutes, 10 to 20% acetonitrile gradient in 60 minutes, 30 to 40% acetonitrile gradient in 40 minutes, and 40 to 60% acetonitrile gradient in 5 minutes. The protein eluted from the column was monitored by measuring the absorbance at the wavelength of 280 nm. When the fraction eluted by the gradient of acetonitrile was analyzed by an SDS-PAGE analysis and a Western blotting analysis, it was found that NucB1-N77 was eluted at the acetonitrile concentration of 37%. Therefore, the fraction was recovered and NucB1-N77 was obtained.

Human/mouse/rat-derived NucB1-N77 each are represented by SEQ ID NOs: 17 to 19. Further, each recombinant NucB1-N77 has a structure in which “Gly-Ser” remains at the N-terminus.

The recombinant NucB1-N77 of mouse and rat were also prepared similarly. The used templates and primers are as follows.

TABLE 3

| | mouse | rat |
|---|---|---|
| templates | cDNA obtained by | rat Brain cDNA(Clontech |
| | synthesizing from mouse | Corporation) |
| | Hypothalamus mRNA (Clontech | |
| | Corporation) using Super | |
| | Script III (Invitrogen | |
| | Corporation) | |
| 1st PCR Forward Primer | mNucB1-F009: SEQ ID NO: 36 | ratNucB1-F001: SEQ ID NO: 46 |
| 1st PCR Reverse Primer | mNucB1-R1406: SEQ ID NO: 37 | ratNucB1-R1402: SEQ ID NO: 47 |
| 2nd PCR Forward Primer | mNucB1-F094: SEQ ID NO: 38 | rNucB1-F078: SEQ ID NO: 48 |
| 2nd PCR Reverse Primer | mNucB1-R301: SEQ ID NO: 39 | rNucB1-R285: SEQ ID NO: 49 |

### Example 4

### Preparation of Recombinant NucB1 by Recombinant Technology

In order to prepare NucB1 having a high homology with NucB2 in large scale, a recombinant human NucB1 was prepared by using recombinant technology. Recombinants NucB1 of rat and mouse were also prepared as in the above. Specifically, a gene encoding human NucB1 was obtained, and at the N-terminus a gene of GST (glutathione-S-transferase) and a histidine tag was bound, and then an expression vector was constructed so that a cleavage site (-Leu-Val-Pro-Arg-Gly-Ser-) by thrombin may intervene between an amino acid sequence of a histidine tag and an amino acid sequence of the human NucB1 in a protein after translation. The details are as follows.

A gene of the human NucB1 was obtained by performing PCR (Nested PCR) twice using as a template cDNA synthesized from human Hypothalamus mRNA (Clontech Laboratories) using Super Script III (Invitrogen Corporation).

By using 0.5 μL of the PCR product obtained by the first round PCR in Example 3 as a template, the second round PCR was performed under the same conditions and using the same reagent as in the second round PCR in Example 1 except for using the following forward (hNucB1-F096 [Sac2Thr]) and reverse (hNucB1-R1376[NotI]) primers.

Forward Primer(hNucB1-F096[Sac2Thr]):
(SEQ ID NO: 27)
5′-GGTTCCGCGGGTCTGGTTCCGCGTGGTTCTGTCCCCCTGGAGCG
AGGGGCGCCCAAC-3′
Reverse Primer (hNucB1-R1376[NotI]):
(SEQ ID NO: 26)
5′-GGTTGCGGCCGCTCACAGATGCTGGGGCACCTCAACCTCA-3′

PCR reaction sample after performing the second round PCR was purified by phenol/chloroform extraction, and then cleaved by restriction enzymes SacII and NotI. The fragments were subjected to agarose gel electrophoresis, and then a band corresponding to the length of around 1.2K by was cut out, and purified using a QIAEX-II kit (QIAGEN Inc.). The purified PCR product with around 1.2K by was subjected to ligation using a Quick DNA ligase kit (New England Biolabs, Inc.) to a pET41a (+) plasmid vector (Novagen) cleaved by restriction enzymes SacII and NotI. The ligated vector was introduced into an Escherichia coli strain JM109, a small-scale plasmid extraction was performed with the obtained two transformants. By using the obtained plasmid, a base sequence analysis of the incorporated human NucB1 gene sequence was performed as in Example 1. As a result, it was confirmed that an expression vector in which a gene having a correct human NucB1 sequence was incorporated was obtained. This was named “pET41a(+)GST-His-LVPRGS-hNucB1 full”.

By using the obtained pET41a(+)GST-His-LVPRGS-hNucB1 full, a fusion protein (GST-His-LVPRGS-hNucB1 full) of GST/histidine tag/thrombin cleavage sequence/human NucB1 was expressed and recovered as in Example 1. Further, as in Example 1, hNucB1 full was obtained by removing the portion of the GST and histidine tag from the fusion protein (GST-His-LVPRGS-hNucB1 full), treating with thrombin and purifying by using reversed-phase chromatography. The gradient of acetonitrile was used setting the same as in Example 1. When the fraction eluted by the gradient of acetonitrile was analyzed by an SDS-PAGE analysis and a Western blotting analysis, it was found that hNucB1 full was eluted at the acetonitrile concentration of around 45%. Therefore, the fraction was recovered to obtain hNucB1 full.

The recombinant NucB1 of mouse and rat were also prepared similarly. The used templates and primers are as follows.

TABLE 4

| | mouse | rat |
|---|---|---|
| templates | cDNA obtained by | rat Brain cDNA(Clontech |
| | synthesizing from mouse | Corporation) |
| | Hypothalamus mRNA (Clontech | |
| | Corporation) using Super | |
| | Script III (Invitrogen | |
| | Corporation) | |
| 1st PCR Forward Primer | mNucB1-F009: SEQ ID NO: 36 | ratNucB1-F001: SEQ ID NO: 46 |
| 1st PCR Reverse Primer | mNucB1-R1406: SEQ ID NO: 37 | ratNucB1-R1402: SEQ ID NO: 47 |
| 2nd PCR Forward Primer | mNucB1-F094: SEQ ID NO: 38 | rNucB1-F078: SEQ ID NO: 48 |
| 2nd PCR Reverse Primer | mNucB1-R1360: SEQ ID NO: 40 | rNucB1-R1357 : SEQ ID NO: 50 |

### Example 5

### Preparstion of Rabbit Polyclonal Antibody Against NESFATIN Partial Peptide

### (1) Preparation of Antibody

In order to prepare an antibody against NESFATIN, the N-terminus and C-terminus of an amino acid sequence (SEQ ID NO: 9) of human NESFATIN polypeptide, and the C-terminus of human NESFATIN-1 and 4 types of synthetic peptides (SEQ ID NOs: 1 to 4, synthesized by SIGMA genosys Ltd.) of partial internal amino acid sequence were used as an antigen (hereinafter, the peptide is collectively referred to NAP peptide).

Nesfatin-1 C-terminus peptide (NSF1-C18):
(SEQ ID NO: 1)
N-terminus-GCSKELDLVSHHVRTKLDEL-C-terminus
Nesfatin-1 Middle peptide (NSF1-M15):
(SEQ ID NO: 2)
N-terminus-PDTGLYYDEYLKQVIC-C-terminus
Nesfatin C-terminus peptide (NSF-C18):
(SEQ ID NO: 3)
N-terminus-GCQGIPPSGPAGELKFEPHI-C-terminus
Nesfatin N-terminus peptide (NSF-N19):
(SEQ ID NO: 4)
N-terminus-VPIDIDKTKVQNIHPVESAC-C-terminus

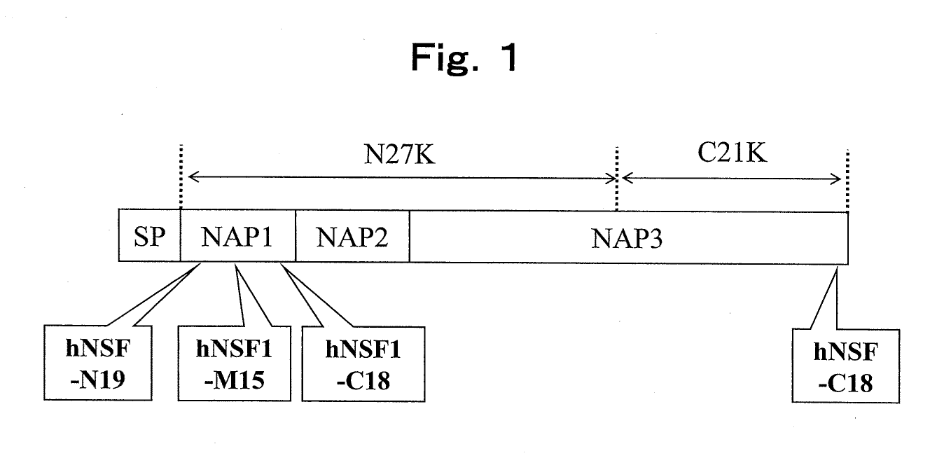

Partial antigen peptide is illustrated in FIG. 1.

The synthetic NAP peptide was obtained by conjugating a peptide to Keyhole Limpet Hemocyanin (KLH) using Imject (registered trademark) Maleimide Activated Mariculture Keyhole Limpet Hemocyanin from PIERCE, in accordance with the attached protocol. A 0.2-mg aliquot of the obtained conjugate was used for one dose for immunizing one rabbit. A 0.25-ml aliquot of the conjugate solution (at a conjugate concentration of 1 mg/mL) and equal parts of complete Freund's adjuvant H-37Ra (Cat. No. 528-00031, Wako Pure Chemical Industries-Difco) were mixed, and the immunization performed by intradermally injecting the resultant mixture by 50 μL to 8 sites in the back of shaved New Zealand white rabbit (purchased from Imai Experimental Animal Farm). The same immunization was further performed once every 2 weeks 4 times, and one week after the last immunization a part of blood was collected, and then antibody titers in the serum were confirmed by ELISA using an immunized peptide conjugate and on the next day the animal was sacrificed to collect the whole blood. Serum was prepared from the obtained blood, and rabbit IgG was purified from the serum using DEAE Sepharose FF (Cat. No. 17-0709-10, Amersham Bioscience) by a conventional method. The purified rabbit IgG was affinity-purified using 1 mg of NAP peptide with the use of a peptide-immobilized column prepared by a SulfoLink kit (Cat. No. 44895, PIERCE) in accordance with the protocol attached to the kit. By using hNSF-C18, hNSF-N19, hNSF1-C18 and hNSF1-M15 as an immunogen, antibody Nos. 4993 and 4994, antibody Nos. 4995 and 4996, antibody Nos. 4997 and 4998, and antibody Nos. 5036 and 5037, were prepared respectively.

### (2) Reactivity Assessment of Antibody by Antigen ELISA

By using the obtained antiserum or antigen affinity-purified antibody, reactivity assessment of each antibody was performed by antigen ELISA against human Nesfatin-1 (human NSF1), human Nesfatin (human NSF full), human NucB1-N77, human NucB1, rat Nesfatin-1 (rat NSF1), rat Nesfatin (rat NSF full), rat NucB1-N77 and rat NucB1 (rat NucB1 full).

GST-fused recombinant of each human Nesfatin-1, human Nesfatin, human NucB1-N77, human NucB1, rat Nesfatin-1, rat Nesfatin, rat NucB1-N77 and rat NucB1, which was diluted with PBS (pH 7) to 1 μg/mL, was respectively added by 50 μL onto a 96-well ELISA plate (MaxiSorp: Nunc), and reacted overnight at 4° C. The resultant plate was washed with TBS containing 0.05% Tween20 three times, and blocked with PBS containing 3% bovine serum albumin (BSA) for one hour at 37° C. The resultant was washed with TBS containing 0.05% Tween20 three times, and added with 50 μL, of antiserum diluent (dilution series prepared by serially diluting the antiserum concentration by 2-fold from 400-fold dilution) or a diluent of antigen affinity-purified antibody (dilution series prepared by serially diluting the antibody concentration by 2-fold from 1 μg/mL with PBS containing 3% bovine serum albumin (BSA)) and the resultant was reacted for one hour at room temperature. The resultant was washed with TBS containing 0.05% Tween20 three times, and added with 50 μL, of an alkaline phosphatase binding goat anti-rabbit IgG antibody (Biosource Corp.) diluted with PBS containing 3% BSA to 2000-fold and the resultant was reacted for one hour at room temperature. The resultant was washed with TBS containing 0.05% Tween20 three times, and added with 100 μL solution of the 1 mg/mL PNPP (p-nitrophenylphosphate, Wako Pure Chemical Industries, Cat. No. 149-02342) dissolved in 0.1 M diethanolamine (pH 10.0), and the resultant was reacted for one hour at room temperature, and then the absorbance of the resultant was measured at 405 nm.

The result of the reactivity assessment for each human recombinant protein of each antibody is shown in Table 5. The reactivity was assessed in 4 levels.

⊚ (Excellent): OD (405 nm) of 2.4 or more
◯ (Good): OD (405 nm) of 1.0 or more to less than 2.4
Δ (Fair): OD (405 nm) of 0.4 or more to less than 1.0
x (Poor): OD (405 nm) of less than 0.4

TABLE 5

| | Antibody | Human | | | |
|---|---|---|---|---|---|
| Immunogen | No. | NSF1 | NSF full | NucB1-N77 | NucB1 full |
| hNSF-C18 | 4993 | X | ⊚ | X | X |
| | 4994 | X | ⊚ | X | X |
| hNSF-N19 | 4995 | ◯ | ◯ | X | X |
| | 4996 | ◯ | Δ | X | X |
| hNSF1-C18 | 4997 | ⊚ | X | ⊚ | X |
| | 4998 | ⊚ | X | ⊚ | X |

The result of the reactivity assessment for each rat recombinant protein of each antibody is shown in Table 6. The reactivity was assessed in 4 levels.

⊚ (Excellent): OD (405 nm) of 1.5 or more
◯ (Good): OD (405 nm) of 1.0 or more to less than 1.5
Δ (Fair): OD (405 nm) of 0.2 or more to less than 1.0
x (Poor): OD (405 nm) of less than 0.2

TABLE 6

| | Antibody | Rat | | | |
|---|---|---|---|---|---|
| Immunogen | No. | NSF1 | NSF full | NucB1-N77 | NucB1-full |
| hNSF-C18 | 4994 | X | ⊚ | X | Δ |
| hNSF-N19 | 4996 | X | X | X | X |
| hNSF1-C18 | 4998 | ⊚ | X | ⊚ | X |
| hNSF1-M15 | 5036 | ◯ | Δ | X | X |
| | 5037 | ◯ | Δ | X | X |

(3) Confirmation of reactivity of antibody by Western Blotting

Reactivity of an anti-Nesfatin polyclonal antibody obtained by using recombinant Nesfatin-1 or Nesfatin to recombinant Nesfatin-1 or Nesfatin of human or rat was confirmed. Each recombinant protein aqueous solution was equivalently mixed with Laemmli sample buffer (BIO-RAD, Cat. No. 161-0737) containing 5% β-mercaptoethanol for SDS-polyacrylamide gel electrophoresis (SDS-PAGE) to prepare as sample, the resultant was electrophoresed at 100V for one hour using Tris-glycine/SDS buffer (25 mM Tris, 192 mM glycine, 0.1% SDS, pH 8.3) at 5 pmole/lane in polyacrylamide gels (Ready Gel, 10-20%, Cat. No. 161-J390V, BIO-RAD), and then the result was transferred to a nitrocellulose membrane. This membrane was blocked with 3% gelatin/TBS for one hour at room temperature, and washed with TBS-0.05% Tween three times and with TBS once. After this, in 1% gelatin/TBS solution containing 1 μg/mL of anti-Nesfatin polyclonal antibody, reaction was performed overnight at room temperature. The next day, the resultant was washed with TBS-0.05% Tween three times and with TBS once, an anti-rabbit IgG goat polyclonal antibody peroxidase conjugate (Cat. No. 674371, Cappel) diluted in 1% gelatin/TBS solution at a concentration of around 1 μg/mL was reacted for one hour at room temperature. Next, the resultant was washed with TBS-0.05% Tween three times and with TBS twice, and made the color develop by using a HRP color development kit (Cat. No. 170-64631, BIO-RAD) for 5 minutes at room temperature to detect the band. As a result, the reactivity to recombinant Nesfatin or Nesfatin-1 was confirmed.

### Example 6

### Preparation of Rabbit Polyclonal Antibody Against Recombinant NESFATIN or Nesfatin-1

### (1) Acquisition of Anti-Recombinant NESFATIN Antibody

Recombinant Nesfatin, Nesfatin-1, and Nesfatin-C21K (each 40 μg at one time) prepared in Examples 1 and 2 were intradermally administered together with Freund's complete adjuvant (1:1, manufactured by BACTO) into two rabbits, respectively, once every two weeks (an antigen recombinant protein region is shown in FIG. 1). Intradermal administration was performed 4 times and then the whole blood was collected to obtain antiserum. From the serum, according to an ordinary method, IgG was purified by using a protein A-Sepharose 4B column (manufactured by Pharmacia), the purified rabbit IgG was affinity-purified by using 1 mg of a recombinant Nesfatin or Nesfatin-1 with the use of a recombinant Nesfatin- or Nesfatin-1-immobilized column prepared using a SulfoLink kit (Cat. No. 44895, PIERCE) in accordance with the protocol attached to the kit. Thus, anti-Nesfatin polyclonal antibody (anti-Nesfatin PAb) and anti-Nesfatin-1 polyclonal antibody (anti-Nesfatin-1 PAb) were obtained. Specifically, by using rNSF1, rNSF full and rNSF C21K as an immunogen, antibody Nos. 6151 and 6152, antibody Nos. 6153 and 6154, and antibody Nos. 6155 and 6156, were prepared respectively.

### (2) Reactivity Assessment of Antibody by Antigen ELISA

By using the obtained antigen affinity-purified antibody, a reactivity assessment of each antibody was performed by antigen ELISA against human Nesfatin-1 (human NSF1), human Nesfatin (human NSF full), human NucB1-N77, human NucB1 (human NucB1-full), rat Nesfatin-1 (rat NSF1), rat Nesfatin (rat NSF full), rat NucB1-N77 and rat NucB1 (rat NucB1 full) as in Example 5 (2). Dilution series was prepared by serially diluting the antibody concentration by 2-fold from 1 μg/mL with PBS containing 3% bovine serum albumin (BSA), and reactivity of the antigen affinity-purified antibody was assessed.

The result of the reactivity assessment for each recombinant protein of each antibody is shown in Table 7. The reactivity was assessed in 4 levels.

⊚ (Excellent): OD (405 nm) of 2.0 or more
◯ (Good): OD (405 nm) of 1.0 or more to less than 2.0
Δ (Fair): OD (405 nm) of 0.15 or more to less than 1.0
x (Poor): OD (405 nm) of less than 0.15

TABLE 7

| | | Human | | | | Rat | | | |
|---|---|---|---|---|---|---|---|---|---|
| | Antibody | | | NucB1- | NucB1 | | | NucB1- | NucB1- |
| Immunogen | No | NSF1 | NSF full | N77 | full | NSF1 | NSF full | N77 | ful |
| hNSF1 | 6151 | ⊚ | ◯ | ⊚ | X | ⊚ | Δ | ⊚ | X |
| | 6152 | ⊚ | ◯ | ⊚ | X | ⊚ | ⊚ | ⊚ | X |
| hNSF full | 6153 | Δ | ⊚ | X | Δ | Δ | ⊚ | X | Δ |
| | 6154 | ⊚ | ⊚ | X | Δ | Δ | ⊚ | X | Δ |
| hNSF C21K | 6155 | X | ⊚ | X | ◯ | X | ⊚ | X | ◯ |
| | 6156 | X | ⊚ | X | ⊚ | X | ⊚ | X | ⊚ |

### (3) Confirmation of Reactivity by Western Blotting

Reactivity of an anti-Nesfatin polyclonal antibody obtained by using recombinant Nesfatin-1 or Nesfatin was confirmed as in Example 5 (3). The confirmation was performed as in Example 5 (3) except for using the antibody obtained in Example 6 (1) as an antibody to be used. As a result, the reactivity to recombinant Nesfatin or Nesfatin-1 was confirmed.

### Example 7

### Construction of Sandwitch ELISA(=Enzyme-Linked Immunosorbent Assay) for Nesfatin Using Rabbit Polyclonal ANTIBODY AGAINST NESFATIN

### (1) Construction of Nesfatin Measurement System

### (A) Preparation of Biotinylated Antibody

An antigen affinity-purified anti-recombinant human Nesfatin-1 rabbit polyclonal antibody (antibody Nos. 4994, 6151, 6152 or 6153) (IgG) obtained in Example 6 was biotinylated as follows. 200-fold molar equivalent of biotinylating reagent was added into an IgG solution (PBS solution) at 1 to 3 mg/mL, and the resultant solution was reacted for one hour at room temperature. As the biotinylating reagent, Sulfo-NHS-LC-Biotin (#21335, molecular weight: 556, manufactured by PIERCE) was dissolved with dimethylformamide to 50 mM and used. Then, monoethanolamine (pH 8.0) was added so as to obtain the final concentration of 10 mM, the resultant was further reacted for one hour at room temperature. The resultant reaction solution was applied to a Sephadx G-25 gel filtration column and discharged with the PBS(−) to recover a biotinylated IgG. The recovered biotinylated IgG was added with BSA at a final concentration of 1% and NaN3 at a final concentration of 0.1%, and stored.

### (B) Preparation of Antibody-Immobilized Plate

A PBS solution containing 2 μg/mL of an antibody (antigen affinity-purified anti-Nesfatin C-terminus peptide rabbit polyclonal antibody (antibody No. 4994)) prepared in Example 5 was added by 50 μL into each well of a 96-well plate (MaxiSorp: Nunc), and the resultant plate was incubated overnight at a temperature of 4° C. The resultant was washed with TBS-0.05% Tween20, added into each well by 250 μL of PBS solution containing 3% bovine serum albumin (BSA), and incubated for one hour at 37° C. to obtain an antibody-immobilized plate on which antibody No. 4994 was immobilized.

### (C) Construction of Sandwitch ELISA

A sandwitch ELISA using an anti-Nesfatin IgG (antibody No. 4994)-immobilized plate prepared in Example 7 (1) (B) and a biotinylated antibody prepared in Example 7 (1) (A) was examined.

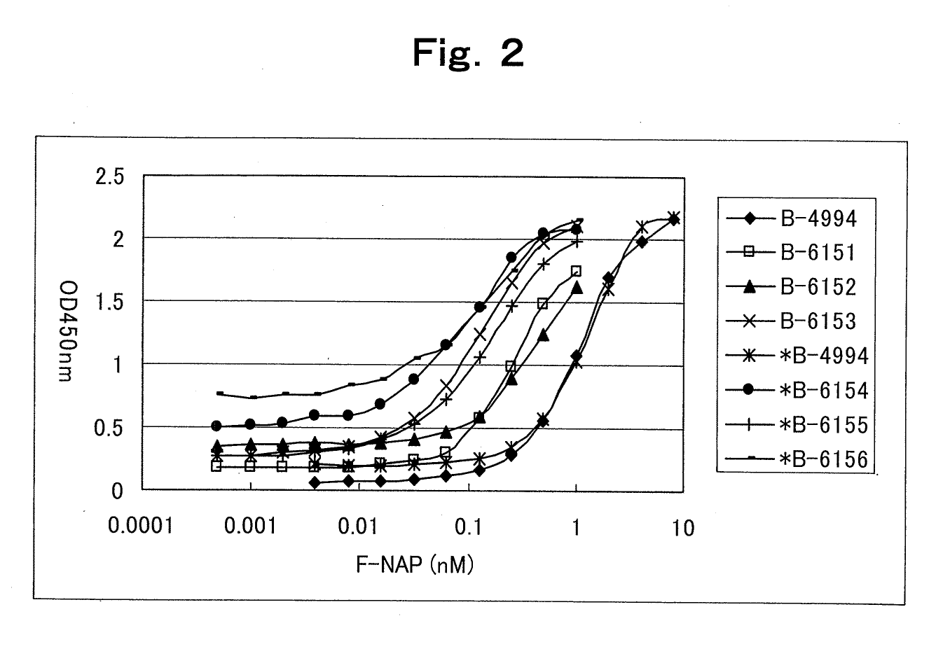

50 μL of 3% BSA-containing 10 mM PBS (pH 7.2) solution (test substance diluent) containing purified recombinant human Nesfatin (F-NAP) (standard substance) in the range of 0 to 2 nM (=0 to 100 pg/mL) was added onto an anti-NESFATIN IgG-immobilized plate prepared in the above-mentioned Example 7 (1) (B), and the resultant plate was incubated for one hour at room temperature, and washed with TBS-0.05% Tween20 three times, and then 50 μL of 3% BSA-containing PBS (pH 7.2) solution containing at 2 μg/mL the biotinylated IgG prepared in the above-mentioned Example 7 (1) (A) was added and the resultant was incubated for one hour at room temperature. Next, after washing with TBS-0.05% Tween20, 3% BSA-containing 10 mM PBS (pH 7.2) solution in which streptavidin labeled peroxidase (#SNN1004, manufactured by Biosource Corp.) was diluted 10,000-fold was added at 50 μL/well, and the resultant was incubated for one hour at room temperature. After this, the resultant was washed with TBS-0.05% Tween20, 0.1M phosphate/citrate buffer (pH 4.3) (TMB Peroxidase EIA Substrate Kit, Bio-Rad) containing 3,3′,5,5′-tetramethylbenzidine hydrochloride and 2.5 mM 0.02% H2O2 was added into each well by 50 μL, the resultant solution was reacted for 5 minutes at room temperature, and then 1 M sulfuric acid aqueous solution was added by 50 μL as a reaction terminator to terminate the enzyme reaction. Next, an absorption intensity of this solution was measured at the wavelength of 450 nm by using a spectrophotometer, and was blotted responding to a standard substance concentration of 0 to 2 nM (=0 to 100 pg/mL). The results are shown in FIG. 2.

### (2) Assessment of the Sensitivity to Species Difference in a Sandwitch ELISA for Nesfatin

By using an anti-Nesfatin IgG (antibody No. 4994)-immobilized plate prepared in Example 7 (1) (B) and a biotinylated antibody (antibody No. 6151 or 6152) prepared in Example 7 (1) (A), the cross-reactivity with rat or mouse Nesfatin was examined.

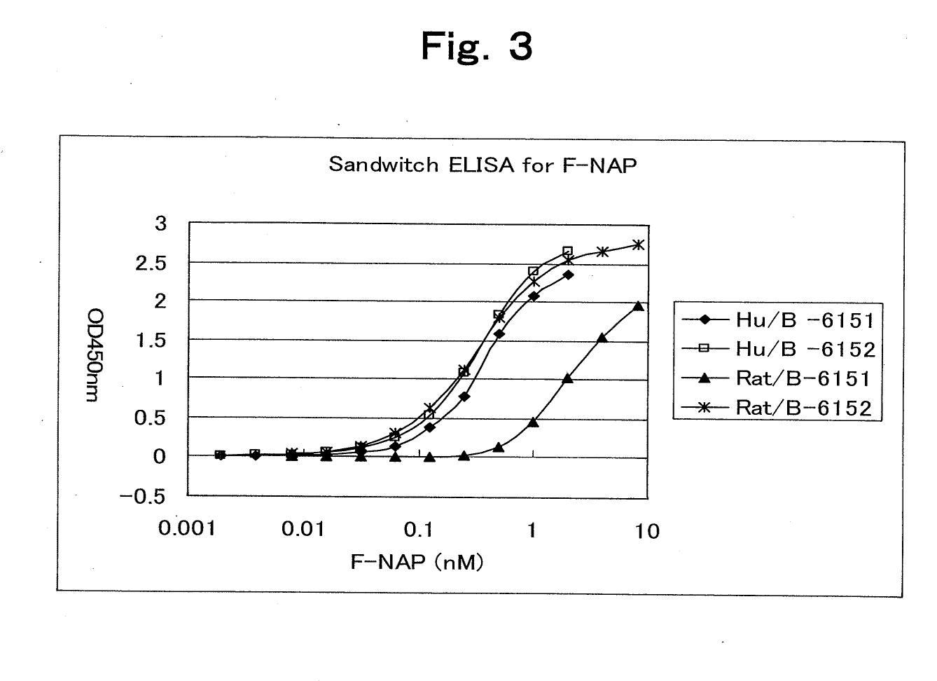

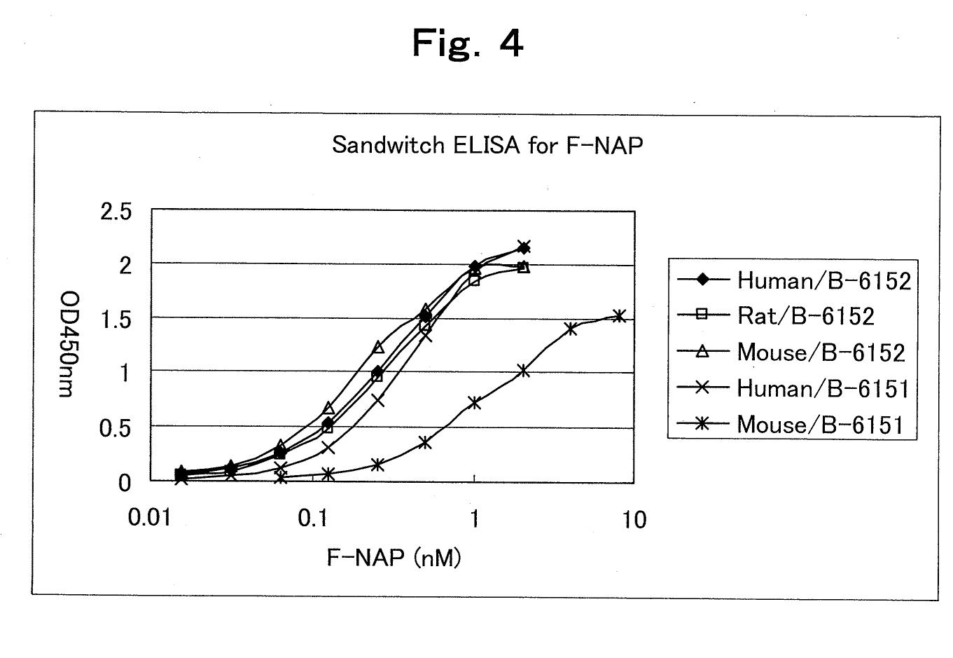

As a test substance, a test substance dilution series of 3% BSA-containing 10 mM PBS (pH 7.2) solution containing purified recombinant human Nesfatin (F-NAP) (standard substance), recombinant rat Nesfatin, or mouse Nesfatin in the range of 0 to 2 nM (=0 to 100 pg/mL) was prepared and used. As for a biotinylated antibody, 3% BSA-containing 10 mM PBS (pH 7.2) solution containing a biotinylated antibody at 2 μg/mL was used. The rest was performed as in Example 7 (1) (C). As for a test substance of each species of Nesfatin at a concentration of 0 to 2 nM (=0 to 100 pg/mL), an absorption intensity of this solution was measured at the wavelength of 450 nm using a spectrophotometer, and was blotted. The results are shown in FIGS. 3 and 4.

As a result, it was found that there has less species difference in the combination of an antibody to use for immobilization: antibody No. 4994 and a biotinylated antibody to use for detection: antibody of No. 6152, and each species of Nesfatin was measured with good sensitivity. On the other hand, it was found that there has high reactivity to human in the combination of an antibody to use for immobilization: antibody No. 4994 and a biotinylated antibody to use for detection: antibody of No. 6151, therefore, the combination is suitable for a measurement of human Nesfatin.

### Example 8

### Construction of Nesfatin-1 Sandwitch ELISA using Rabbit Polyclonal Antibody Against Nesfatin-1

### (1) Construction of Nesfatin-1 Sandwitch ELISA-1

### (A) Preparation of Biotinylated Antibody

Biotinylation of an antigen affinity-purified anti-Nesfatin-1 polyclonal antibody obtained in Example 5 or 6 (antibody No. 4998, antibody No. 6151, or antibody No. 6152) (IgG) was performed as in Example 7 (1) (A).

### (B) Preparation of Antibody-Immobilized Plate

By using an antibody prepared in Example 6 (antigen affinity-purified anti-Nesfatin-1 C-terminus peptide rabbit polyclonal antibody (antibody No. 6151 or antibody No. 6152)), an antibody-immobilized plate on which antibody No. 6151 or antibody No. 6152 was immobilized was prepared as in Example 7 (1) (B).

### (C) Examination for Construction of Sandwitch ELISA-1

A sandwitch ELISA using an anti-Nesfatin-1 IgG (antibody No. 6151 or antibody No. 6152)-immobilized plate prepared in the above-mentioned Example 8 (1) (B) and a biotinylated IgG (antibody No. 6151 or antibody No. 6152) prepared in the above-mentioned Example 8 (1) (A) was examined.

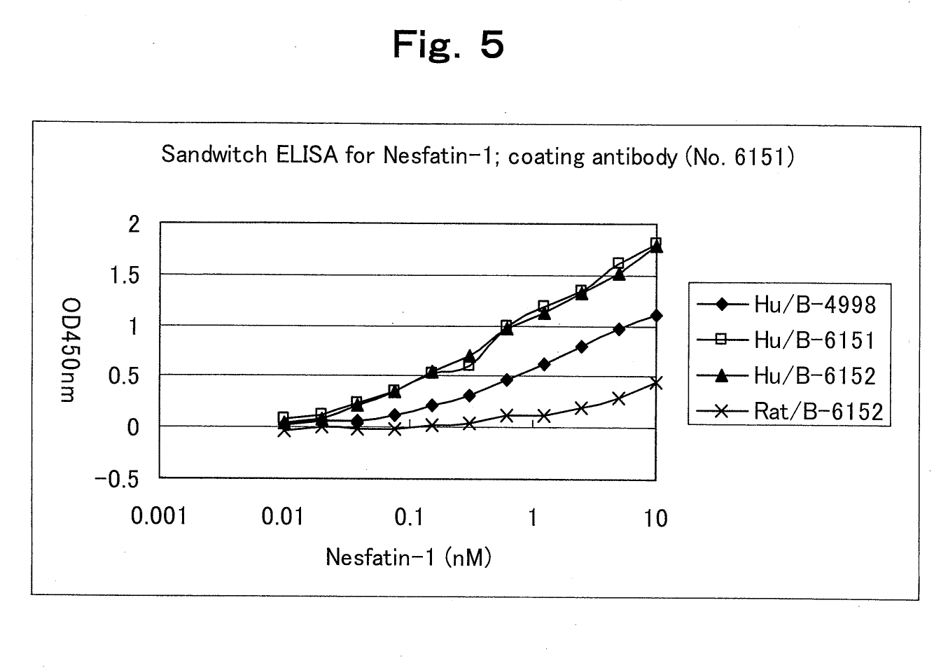

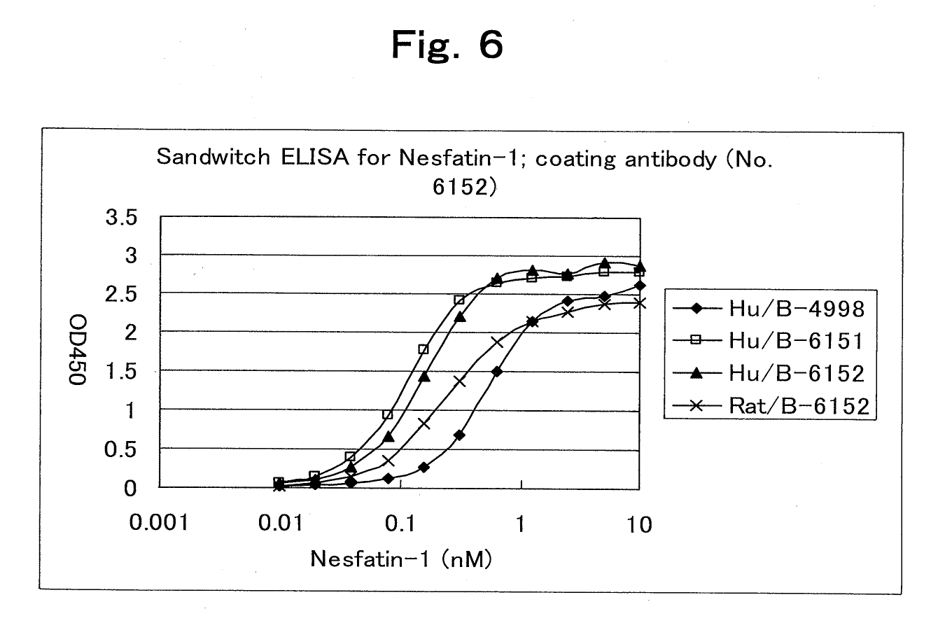

As a test substance, a test substance dilution series of 3% BSA-containing 10 mM PBS (pH 7.2) solution containing purified recombinant human Nesfatin-1 (standard substance) or recombinant rat Nesfatin-1 in the range of 0 to 10 nM was prepared and used. As for a biotinylated IgG, 3% BSA-containing 10 mM PBS (pH 7.2) solution containing 2 μg/mL of biotinylated IgG was used. The rest was performed as in Example 7 (1) (C). As for a test substance at each concentration of 0 to 10 nM, an absorption intensity of this solution was measured at the wavelength of 450 nm, and was blotted. The results are shown in FIGS. 5 and 6.

As a result, it was found that the cross-reactivity of antibody No. 6151 against rat Nesfatin-1 was low and the cross-reactivity of antibody No. 6152 against rat Nesfatin-1 was high. Further, from the above, it was found that antibody of No. 6152 was suitable as a solid phase antibody.

### (D) Examination for Construction of Sandwitch ELISA-2

As a result of Example 8 (1) (C), the cross-reactivity with human NucB1-N77 was examined using an anti-Nesfatin-1 IgG (antibody No. 6152)-immobilized plate prepared in the above-mentioned Example 8 (1) (B) and a biotinylated IgG (antibody No. 6151 or antibody No. 6152) prepared in the above-mentioned Example 8 (1) (A).

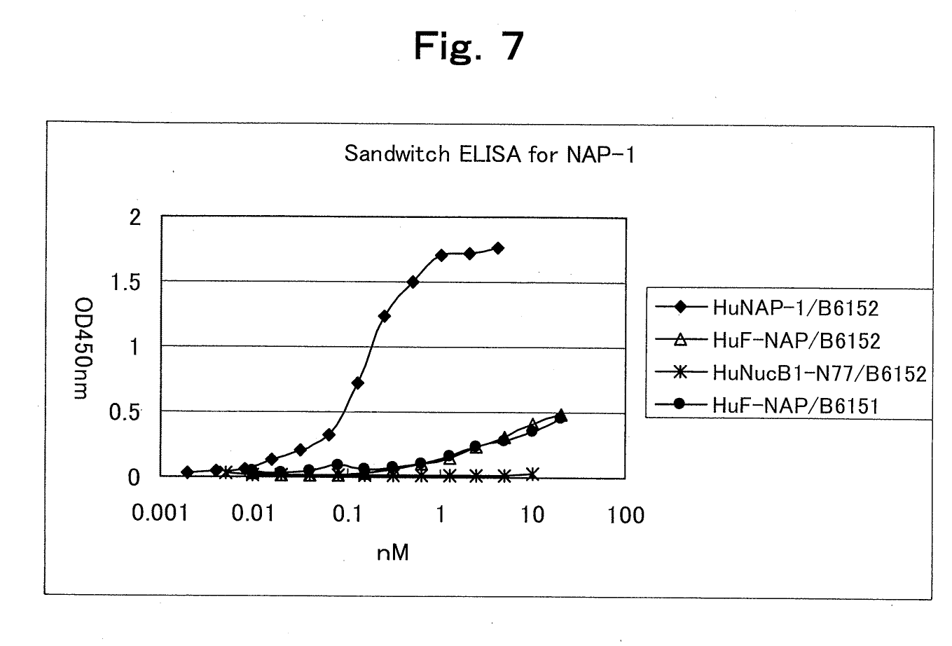

As a test substance, a test substance dilution series of 3% BSA-containing 10 mM PBS (pH 7.2) solution containing purified recombinant human Nesfatin-1 (HuNAP1) (standard substance), recombinant human Nesfatin (HuF-NAP) or recombinant human NucB1-N77 (GST fusion protein) in the range of 0 to 20 nM was prepared and used. As for a biotinylated IgG, 3% BSA-containing 10 mM PBS (pH 7.2) solution containing 2 μg/mL of biotinylated IgG was used. The rest was performed as in Example 7 (1) (C). As for a test substance at each concentration of 0 to 20 nM, an absorption intensity of this solution was measured at the wavelength of 450 nm, and was blotted. The results are shown in FIG. 7.

As a result, in any system using solid-phase antibody No. 6152, detection antibody biotinylated antibody No. 6152 or antibody No. 6151, the cross-reactivity to NucB1-N77 was not observed but the cross-reactivity to Nesfatin was slightly observed, and it was found that the system was incapable of using as a Nesfatin-1 specific sandwitch ELISA.

(2) Construction of Nesfatin-1 sandwitch ELISA-2

### (A) Preparation of Biotinylated Antibody

As in Example 7 (1) (A), an antigen affinity-purified anti-Nesfatin-1 polyclonal antibody (antibody No. 6151 or antibody No. 6152) (IgG) was biotinylated.

### (B) Preparation of Antibody-Immobilized Plate

By using an antibody (antigen affinity-purified anti-Nesfatin-1 C-terminus peptide rabbit polyclonal antibody (antibody No. 4998)) prepared in Example 5, an antibody-immobilized plate on which antibody No. 4998 was immobilized was prepared as in Example 7 (1) (B).

### (C) Construction of Sandwitch ELISA

A sandwitch ELISA using an anti-Nesfatin-1 IgG (antibody No. 4998)-immobilized plate prepared in the above-mentioned Example 8 (2) (B) and a biotinylated IgG (antibody No. 6151 or antibody No. 6152) prepared in the above-mentioned Example 8 (2) (A) was examined.

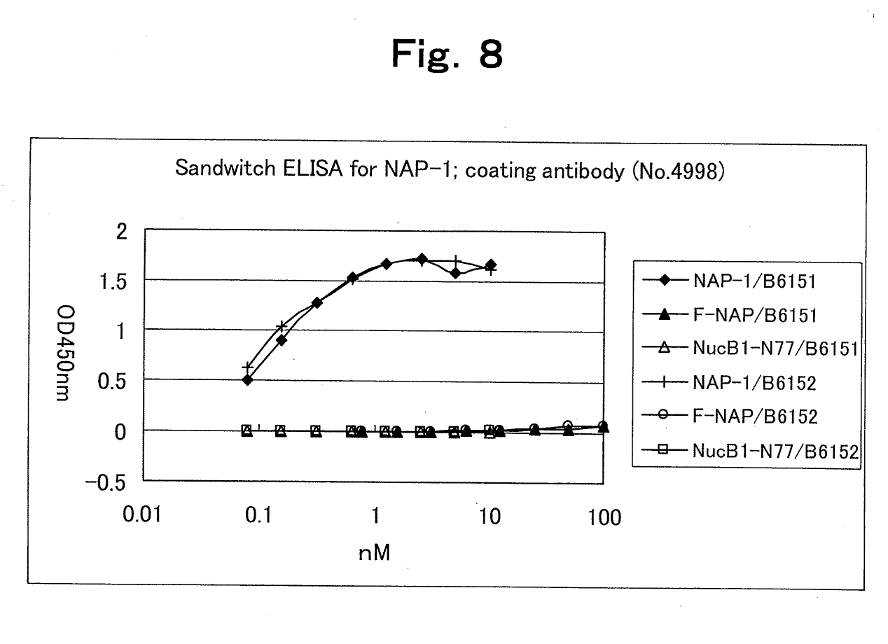

As a test substance, a test substance dilution series of 3% BSA-containing 10 mM PBS (pH 7.2) solution containing purified recombinant human Nesfatin-1 (standard substance) or recombinant human NucB1-N77 in the range of 0 to 10 nM or a test substance dilution series of 3% BSA-containing 10 mM PBS (pH 7.2) solution containing recombinant human Nesfatin in the range of 0 to 100 nM was prepared and used. As for a biotinylated antibody, 3% BSA-containing PBS (pH 7.2) solution containing 2 μg/mL of biotinylated IgG was used. The rest was performed as in Example 7 (1) (C). As for a test substance at each concentration of 0 to 100 nM, an absorption intensity of this solution was measured at the wavelength of 450 nm, and was blotted. The results are shown in FIG. 8.

As a result, it was found that the cross-reactivity to NucB1-N77 or Nesfatin was hardly observed in a system using solid-phase antibody No. 4998, detection antibody biotinylated antibody No. 6151 or antibody No. 6152, the system was capable of using as a Nesfatin-1 specific measurement system.

### (3) Assessment of the Sensitivity for Species Difference in a Sandwitch ELISA of Nesfatin-1 (System of 4998 & B-6152) and Assessment of Cross-Reactivity to Nesfatin

Since the cross-reactivity of antibody No. 6152 to rat was high as a result of the examination described above, the cross-reactivity to rat Nesfatin-1 and mouse Nesfatin-1 were examined by using an anti-Nesfatin-1 IgG (antibody No. 4998)-immobilized plate and a biotinylated antibody (antibody No. 6152).

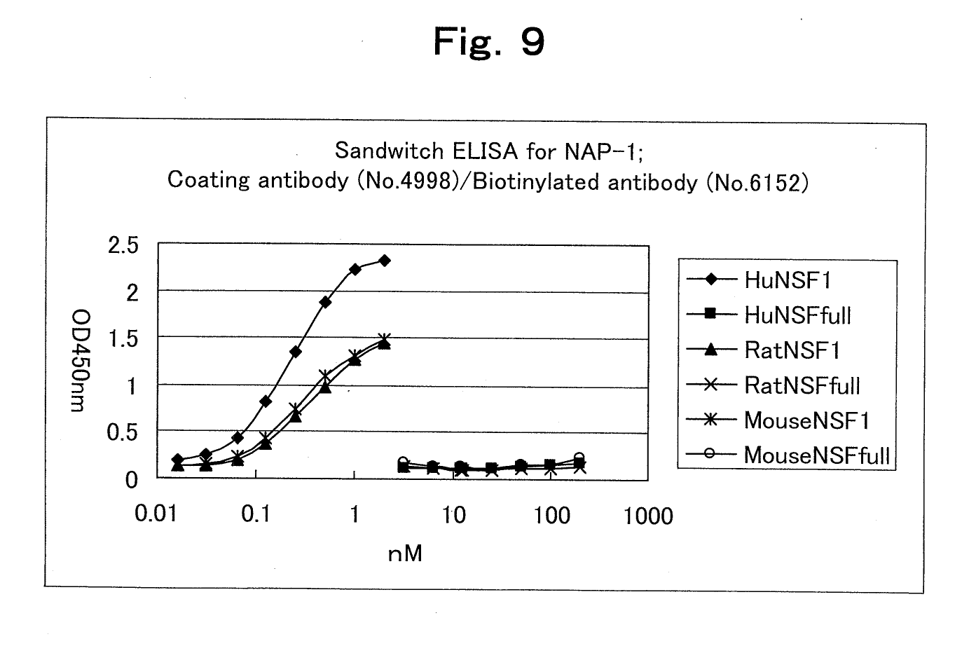

As a test substance, a test substance dilution series of 3% BSA-containing 10 mM PBS (pH 7.2) solution containing purified recombinant human Nesfatin-1 (standard substance), recombinant rat Nesfatin-1, mouse Nesfatin-1, recombinant human Nesfatin, recombinant rat Nesfatin or mouse Nesfatin in the range of 0 to 2 nM was prepared and used. As for a biotinylated IgG (B-6152), 3% BSA-containing 10 mM PBS (pH 7.2) solution containing 2 μg/mL of biotinylated antibody was used. The rest was performed as in Example 7 (1) (C). As for a test substance of each species of Nesfatin at a concentration of 0 to 2 nM, an absorption intensity of this solution was measured at the wavelength of 450 nm, and was blotted. The results are shown in FIG. 9.

As a result, it was found that not only human Nesfatin-1 but also mouse and rat Nesfatin-1 were measured with good sensitivity by using the combination of an antibody to use for immobilization: antibody No. 4998 and a biotinylated antibody to use for detection: antibody No. 6152. In addition, it was found that there was almost no cross-reactivity to human, mouse or rat Nesfatin. From these findings, there was shown the capability that a measurement system of the combination of an antibody to use for immobilization: antibody No. 4998 and a biotinylated antibody to use for detection: antibody No. 6152 was used as a mouse or rat Nesfatin-1 specific sandwitch ELISA.

On the other hand, as for a sandwitch ELISA of the combination of an antibody to use for immobilization: antibody No. 4998 and a biotinylated antibody to use for detection: antibody No. 6151, it was judged from the result in Example 8 (2) (C) that the system was capable of using as a human Nesfatin-1 specific sandwitch ELISA.

### Example 9

### Preparation of Mouse Monoclonal Antibody Against NESFATIN-1>

### (1) Immunization of Mouse by Recombinant Nesfatin

Recombinant human Nesfatin-1, recombinant human Nesfatin, Nesfatin-N27K or Nesfatin-C21K, which was prepared in Example 1 or 2, was intraperitoneally administered by 10 to 20 μg at one time per head together with Freund's complete adjuvant (1:1, manufactured by BACTO) into a Balb/c mouse (a 7-week-old male) once every two weeks. This administration was performed 4 times, and then at the fifth administration, PBS solution of recombinant human Nesfatin-1, recombinant human Nesfatin, Nesfatin-N27K or Nesfatin-C21K was intravenously administered by 50 μg per head on 3 days before the cell fusion.

As for an anti-Nesfatin-1 antibody titer measurement in mouse serum, blood was collected from the tail vein of a mouse and incubated for 30 minutes at 37° C., and then centrifuged at 3000 rpm for 10 minutes to recover the serum, anti-Nesfatin-1 antibody titers in the mouse serum were measured by antigen ELISA. The method is described in the following.

The recombinant human Nesfatin-1 diluted with PBS to 1 μg/mL was added by 50 μL onto a 96-well ELISA plate (Falcon3912, Becton, Dickinson and Company), and the resultant plate was reacted overnight at 4° C. The resultant was washed three times with PBS containing 0.05% Tween20, and treated with PBS containing 3% bovine serum albumin (BSA) for one hour at room temperature. After washing with PBS containing 0.05% Tween20 three times, 50 μL of test substance was added and the resultant was reacted for one hour at room temperature. After washing with PBS containing 0.05% Tween20 three times, 50 μL of alkaline phosphatase binding-goat anti-rabbit IgG antibody (AMI3705, Biosource Corp.) diluted to 2000-fold with PBS containing 3% BSA was added and the resultant was reacted for one hour at room temperature. After washing with TBS containing 0.05% Tween20 three times, 0.1 M of Diethanolamine Buffer (pH 10.0) solution of PNPP (p-Nitrophenylphosphate, Wako Pure Chemical Industries) was reacted for one hour at room temperature, and then the absorbance at 405 nm was measured.

### (2) Preparation of Hybridoma by Cell Fusion

A mouse was killed immediately before the cell fusion, the splenocytes from the killed mouse were homogenized in PBS, and the residues were filtered through nylon mesh, and then the resultant was once subjected to a centrifugal washing treatment with PBS. The cell fusion of these obtained splenocytes and mouse myeloma cells (P3×63Ag8U.1) was performed in accordance with an ordinary method (Kohler, Milstein; Nature, 256, 495-497 (1975)).

In other words, 5×10⁷ splenocytes and 5×10⁶ mouse myeloma cells P3×63Ag8U.1 (P3U1) were washed with RPMI 1640 medium, and the resultant was centrifuged at 1500 rpm for 5 minutes to obtain the cell pellets. 1 mL of 35% polyethylene glycol solution (5.75 ml of RPMI 1640 medium+3.5 mL of polyethylene glycol solution+0.75 mL of dimethyl sulfoxide) was added in 2 minutes and the cells were gently floated. The resultant was added with 1 mL of RPMI 1640 medium in two minutes, and further added with 2 mL of RPMI 1640 medium in two minutes. Next, the resultant was added with 4 mL of GIT-HAT medium (95 μM of hypoxanthine, 0.4 μM of aminopterin, 1.6 μM of thymidine, and GIT medium containing 5% FCS) in two minutes, and further added with 8 mL of GIT-HAT medium in two minutes. After incubating the resultant for 30 minutes at 37° C., the resultant solution was dispensed onto one 96-well flat-bottom plate in which around 10⁴ mouse abdominal exudate cells per well was plated, and then cultured at 37° C. in the presence of 5% CO2.

One week later, half of the medium was exchanged with GIT-HT medium (a medium obtained by removing aminopterin from GIT-HAT medium), and then further cultured for around one week at 37° C. in the presence of 5% CO2 to obtain several hybridoma colonies per well.

### (3) Screening of Hybridoma

Two weeks later, screening by a plate coated with recombinant human Nesfatin-1 or recombinant human Nesfatin was performed. PBS solution (1 μg/mL) of recombinant human Nesfatin-1 or recombinant human Nesfatin was dispensed onto a 96-well plate (Falcon Corp., manufactured by PVC) by 50 μL per well, and then the plate was allowed to stand overnight at 4° C. The resultant was washed, added with 3% BSA/PBS by 200 μL per well, and then the resultant was blocked for one hour at 37° C. The resultant was washed again, and added with the culture supernatant by 50 μL per well, allowed to stand for one hour at room temperature, and then the resultant was washed three times with 0.05% Tween/PBS.

Subsequently, a goat anti-mouse IgG-alkali phosphatase conjugate diluted to 2000-fold with 3% BSA/PBS was added by 50 μl per well, allowed to stand for one hour at room temperature. The resultant was washed again, 1 mg/mL solution of disodium p-nitrophenylphosphate (Wako Pure Chemical Industries) dissolved in a 1 M diethanolamine buffer solution (pH 9.8) containing 0.25 mM magnesium chloride was added by 100 μL per well, and the resultant solution was reacted for 30 minutes at room temperature. The absorbance at 405 nm of the resultant was examined with an ELISA reader instrument (Vmax, Molecular Davice Corp.) for a 96-well plate, and the hybridoma secreting the monoclonal antibody binding to the recombinant human Nesfatin-1 or a recombinant human Nesfatin was selected.

### (4) Cloning of Hybridoma

The hybridoma selected as in the above was twice cloned by a limiting dilution method to establish the cells. Specifically, mouse abdominal exudate cells prepared in HT medium at the density of 10⁶ cells/mL were dispensed into each well, to which the hybridoma cells suspended in HT medium were plated at the ratio of 0.5 cell per well. The resultant was cultured in an incubator in the presence of 5% CO2 for two weeks at 37° C. The culture supernatant was screened by the above-mentioned ELISA method, and a single colony was picked up to establish the cells.

Finally, hybridoma producing IgG having the reactivity to recombinant human Nesfatin-1 or recombinant human Nesfatin was cloned only from the hybridoma obtained by immunizing human Nesfatin-1.

### Example 10

### Reactivity Assessment of Mouse Monoclonal Antibody Against NESFATIN-1

In order to assess the reactivity to various types of antigens (human Nesfatin-1, human Nesfatin, human NucB1-N77, rat Nesfatin-1, or rat Nesfatin) of hybridoma producing antibody, the reactivity was assessed by immobilizing the recombinant GST-fusion proteins prepared in Example 1, 2, 3 or 4 to a plate using an antigen ELISA method.

PBS solution (1 μg/mL) of GST-fusion proteins of human Nesfatin-1, human Nesfatin, human NucB1-N77, rat Nesfatin-1 or rat Nesfatin was dispensed onto a 96-well plate (Falcon Corp., manufactured by PVC) by 50 μL per well, and then the plate was allowed to stand overnight at 4° C. The resultant was washed, added with 3% BSA/PBS by 200 μL per well, and then the resultant was blocked for one hour at 37° C. The resultant was washed again, and added with the hybridoma culture supernatant by 50 μL per well, allowed to stand for one hour at room temperature, and then the resultant was washed three times with 0.05% Tween/PBS.

Subsequently, goat anti-mouse IgG-peroxidase diluted to 1000-fold with PBS containing 3% BSA was added by 50 μL per well, allowed to stand for one hour at room temperature. The resultant was washed again, 1 mg/mL solution of disodium p-nitrophenylphosphate (Wako Pure Chemical Industries) dissolved in a 1 M diethanolamine buffer solution (pH 9.8) containing 0.25 mM magnesium chloride was added by 100 μL per well, and the resultant solution was reacted for 30 minutes at room temperature. The absorbance at 405 nm of the resultant was examined with an ELISA reader instrument (Vmax, Molecular Davice Corp.) for a 96-well plate, and the 21 hybridoma clones secreting the monoclonal IgG antibody specifically binding to the recombinant Nesfatin-1 was selected. Ig typing was performed by using an Isotyping Kit manufactured by PIERCE. As for 9 clones among the 21 hybridoma clones, the results of reactivity assessment for each recombinant protein of each monoclonal antibody are shown in Table 8. The reactivity was assessed in 3 levels.

⊚ (Excellent):OD (405 nm) of 0.3 or more
◯ (Good):OD (405 nm) of 0.06 or more to less than 0.3
x (Poor):OD (405 nm) of less than 0.06

TABLE 8

| | Reactivity | | | | | Purification | |
|---|---|---|---|---|---|---|---|
| | Human | | | | | method | |
| Hybridoma | | | NucB1- | Rat | | Protein G | |
| clone | Nesfatin-1 | Nesfatin | N77 | Nesfatin-1 | Nesfatin | Purification | Ig Type |
| NAP31 | ◯ | X | X | X | X | | IgM |
| NAP33 | ⊚ | X | ⊚ | ⊚ | X | ⊚ | IgG |
| NAP34 | ⊚ | X | ⊚ | ⊚ | X | | IgM |
| NAP35 | ⊚ | X | ⊚ | ⊚ | X | | IgM |
| NAP37 | ⊚ | X | ⊚ | X | X | ⊚ | IgG |
| NAP38 | ⊚ | X | ⊚ | ◯ | X | | IgM |
| NAP39 | ⊚ | X | ◯ | X | X | ⊚ | IgG |
| NAP40-2 | ⊚ | X | X | ⊚ | X | ⊚ | IgG |
| NAP41 | ⊚ | X | ⊚ | ⊚ | X | ⊚ | IgM |

From the above results, 3 clones, that is, NAP37, NAP39 and NAP40-2 were selected from the 21 hybridoma clones producing anti-Nesfatin-1 antibody, cultured in large scale, and then the culture supernatant was recovered. Antibodies in each culture supernatant were purified by using a protein G column according to an ordinary method.

### Example 11

### Construction of Nesfatin-1 sandwitch ELISA using Mouse Monoclonal Antibody Against Nesfatin-1>

### (1) Construction of Nesfatin -1 Sandwitch ELISA

### (A) Preparation of Biotinylated Antibody

IgG of anti-Nesfatin or Nesfatin-1 rabbit polyclonal antibody (antibody No. 4998, antibody No. 5036, antibody No. 5037, antibody No. 6151 or antibody No. 6152) obtained in Example 5 or 6, was biotinylated as in Example 7 (1) (A).

### (B) Preparation of Antibody-Immobilized Plate

By using an antibody (an anti-recombinant human Nesfatin-1 mouse monoclonal antibody (NAP40-2, NAP37 or NAP39)) obtained in Example 10, an antibody-immobilized plate on which NAP40-2, NAP37 or NAP39 was immobilized was obtained as in Example 7 (1) (B).

### (C) Construction of Sandwitch ELISA -(1)

A sandwitch ELISA using a plate on which an anti-recombinant human Nesfatin-1 mouse monoclonal antibody (NAP40-2, NAP37 or NAP39) prepared in the above-mentioned Example 11 (1) (B) was immobilized and a biotinylated IgG (biotinylated antibody No. 6151 or biotinylated antibody No. 6152) prepared in the above-mentioned Example 11 (1) (A) was examined.

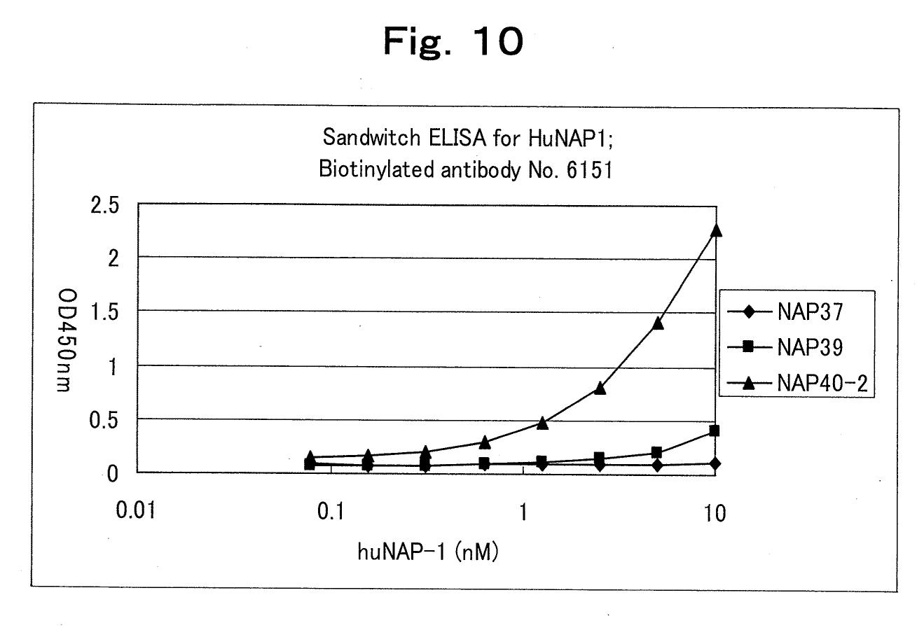

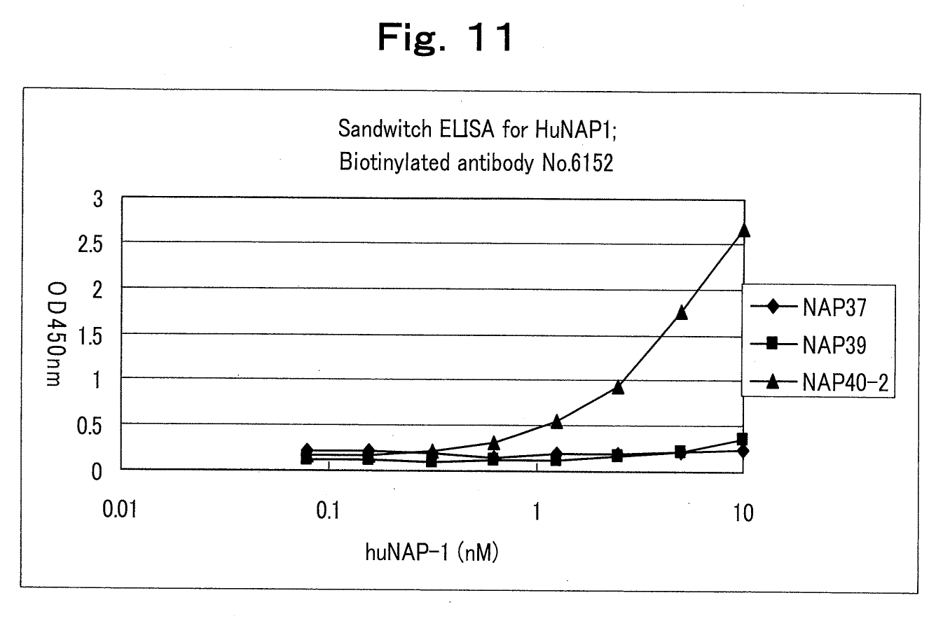

As a test substance, a test substance dilution series of 3% BSA-containing 10 mM PBS (pH 7.2) solution containing purified recombinant human Nesfatin-1 (standard substance) in the range of 0 to 10 nM was prepared and used. As for a biotinylated IgG, 3% BSA-containing 10 mM PBS (pH 7.2) solution containing 2 μg/mL of biotinylated IgG was used. The rest was performed as in Example 7 (1) (C). As for a test substance at each concentration of 0 to 10 nM, an absorption intensity of this solution was measured at the wavelength of 450 nm, and was blotted. The results are shown in FIGS. 10 and 11.

As a result, it was found that human Nesfatin-1 was measured with the highest sensitivity by a NAP40-2 immobilized plate. NAP40-2 was deposited in the Depositary Authority International Patent Organism Depositary, National Institute of Advanced Industrial Science and Technology (Accession number: FERM ABP-10884, Date of the acceptance: Jul. 27, 2007).

### (C) Construction of Sandwitch ELISA -(2)

A sandwitch ELISA using a plate on which an anti-recombinant human Nesfatin-1 mouse monoclonal antibody (NAP40-2) prepared in the above-mentioned Example 11 (1) (B) was immobilized and a biotinylated IgG (biotinylated antibody No. 6151, biotinylated antibody No. 6152, biotinylated antibody No. 4998, biotinylated antibody No. 5036 or biotinylated antibody No. 5037) prepared in Example 11 (1) (A) was examined.

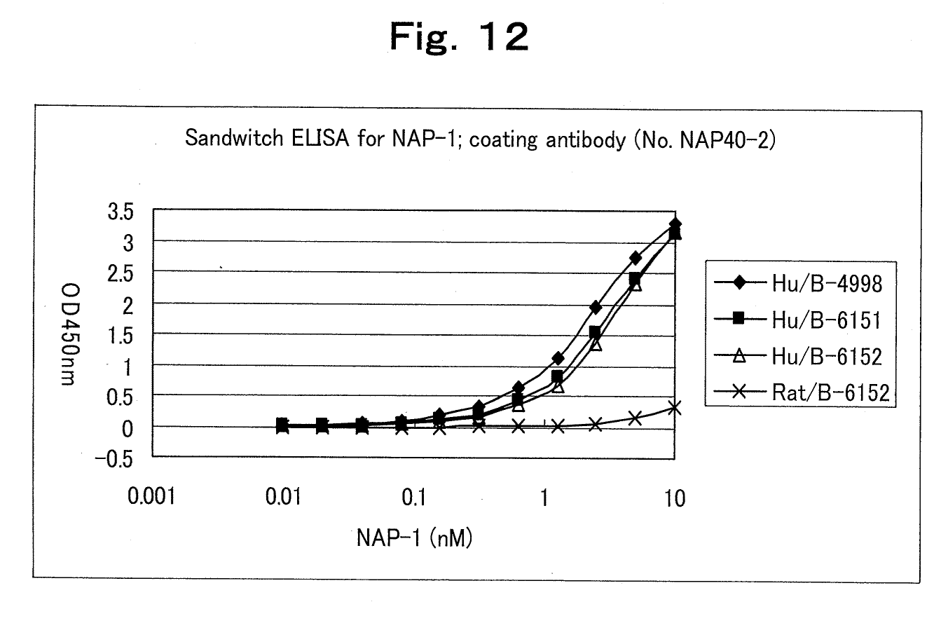

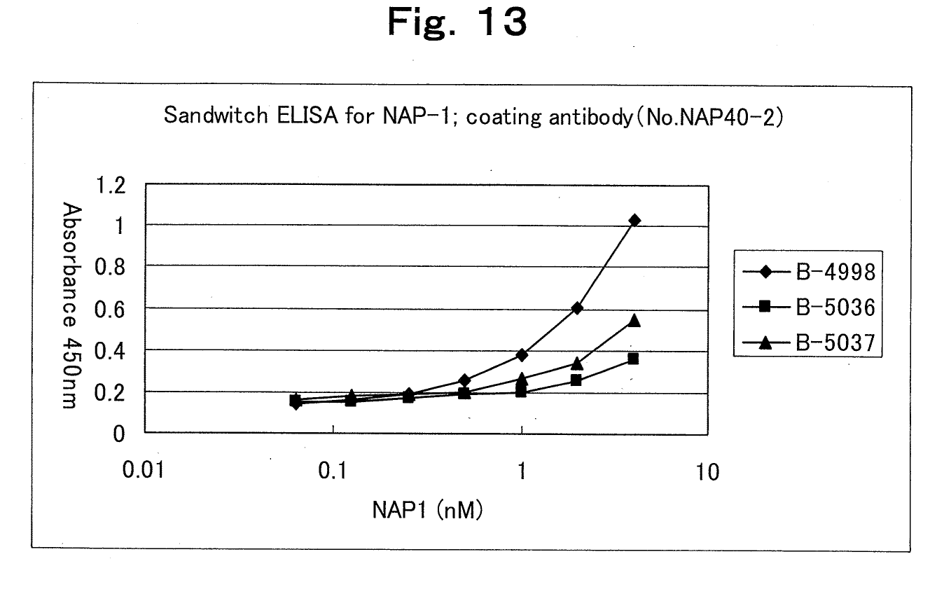

As a test substance, a test substance dilution series of 3% BSA-containing 10 mM PBS (pH 7.2) solution containing purified recombinant human Nesfatin-1 (standard substance) in the range of 0 to 10 nM was prepared and used. As for a biotinylated IgG, 3% BSA-containing 10 mM PBS (pH 7.2) solution containing 2 μg/mL of biotinylated IgG was used. The rest was performed as in Example 7 (1) (C). As for a test substance at each concentration of 0 to 10 nM, an absorption intensity of this solution was measured at the wavelength of 450 nm, and was blotted. The results are shown in FIGS. 12 and 13.

As a result, it was found that human Nesfatin-1 was measured with the highest sensitivity by a biotinylated antibody No. 4998.

### (2) Assessment of the Sensitivity for Species Difference in a Sandwitch ELISA

A cross-reactivity to rat Nesfatin-1 was examined using a sandwitch ELISA (a system using a plate on which anti-recombinant human Nesfatin-1 mouse monoclonal antibody (NAP40-2) was immobilized and a biotinylated antibody No. 4998) that has the highest sensitivity among the systems obtained in the above-mentioned Example 11 (1).

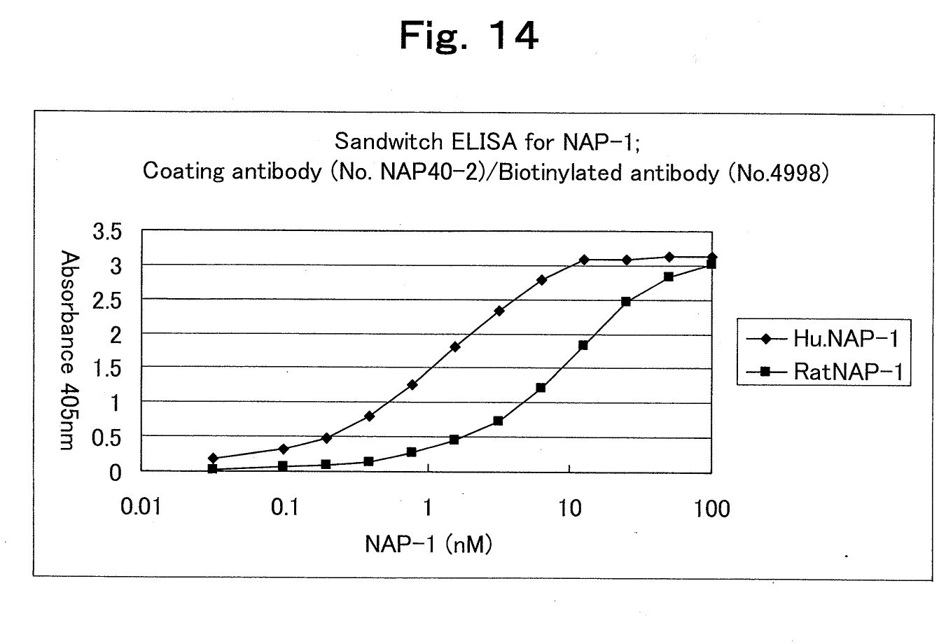

As a test substance, a test substance dilution series of 3% BSA-containing 10 mM PBS (pH 7.2) solution containing purified recombinant human Nesfatin-1 (standard substance) or recombinant rat Nesfatin-1 (standard substance) in the range of 0 to 100 nM was prepared and used. As for a biotinylated IgG, 3% BSA-containing 10 mM PBS (pH 7.2) solution containing 2 μg/mL of biotinylated IgG was used. The rest was performed as in Example 7 (1) (C). As for a test substance at each concentration of 0 to 100 nM, an absorption intensity of this solution was measured at the wavelength of 450 nm, and was blotted. The results are shown in FIG. 14.

### (3) Assessment of Cross-Reactivity to Nesfatin in a Sandwitch ELISA

A cross-reactivity to Nesfatin (full molecule) was examined using a sandwitch ELISA of Nesfatin-1 (a system using a plate on which anti-recombinant human Nesfatin-1 mouse monoclonal antibody (NAP40-2) was immobilized and a biotinylated antibody No. 4998) that has the highest sensitivity among the systems obtained in the above-mentioned Example 11 (1).

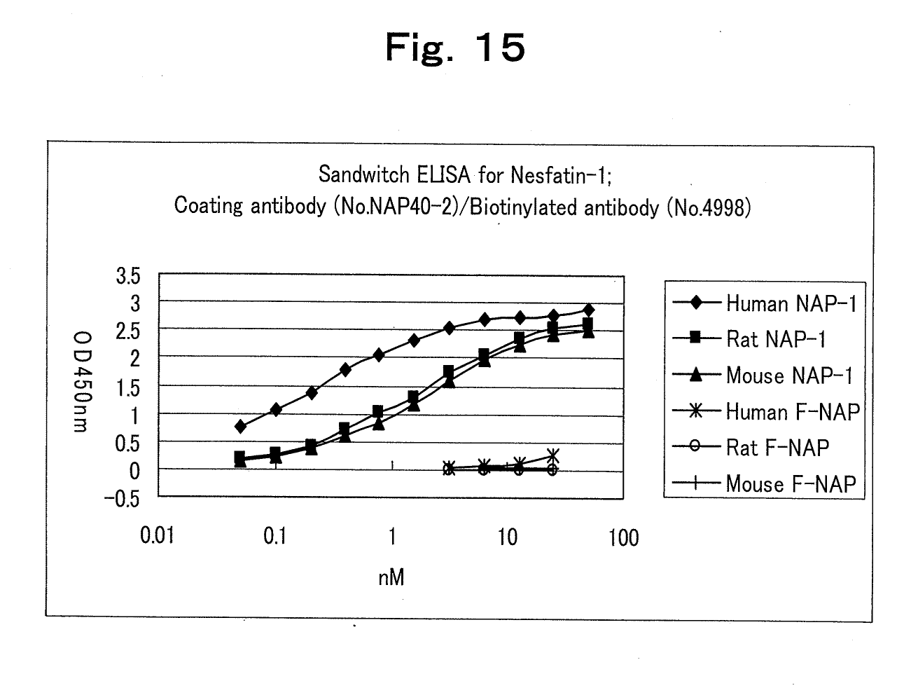

As a test substance, a test substance dilution series of 3% BSA-containing 10 mM PBS (pH 7.2) solution containing purified recombinant human Nesfatin-1 (standard substance), recombinant rat Nesfatin-1 (standard substance) or recombinant mouse Nesfatin-1 (standard substance) in the range of 0 to 50 nM was prepared and used. Further, as a test substance to examine the cross-reactivity, a test substance dilution series of 3% BSA-containing 10 mM PBS (pH 7.2) solution containing purified recombinant human Nesfatin (standard substance), recombinant rat Nesfatin (standard substance), or recombinant mouse Nesfatin (standard substance) in the range of 0 to 25 nM was prepared and used. As for a biotinylated IgG, 3% BSA-containing 10 mM PBS (pH 7.2) solution containing 2 μg/mL of biotinylated IgG was used. The rest was performed as in Example 7 (1) (C). As for a test substance at each concentration of 0 to 50 nM, an absorption intensity of this solution was measured at the wavelength of 450 nm, and was blotted. The results are shown in FIG. 15.

As a result, it was found that the reactivity of rat or mouse to Nesfatin was not detected and the rat or mouse Nesfatin-1 was specifically measured in a system using NAP40-2 antibody immobilization and a biotinylated antibody No. 4998. In addition, since as for the human Nesfatin, human Nesfatin-1 was detected with a nearly 1000-fold high sensitivity although the reactivity was slightly observed at 25 nM, it was judged not to be a problem as long as Nesfatin-1 and Nesfatin are present in the ratio less than that above.

### Example 12

### Preparation of Mouse Monoclonal Antibody Against Nesfatin Partial Peptide

(1) Immunization of Mouse with Nesfatin Partial Peptide

A Nesfatin partial peptide (SEQ ID NOs: 1 to 4) prepared in Example 5 was intraperitoneally administered by 10 to 20 μg at one time per head together with Freund's complete adjuvant (1:1, manufactured by BACTO Corporation) into a Balb/c mouse (a 7-week old male) once every two weeks. This administration was performed 4 times, and then at the fifth administration, 50 μg of solution of recombinant human Nesfatin-1 or recombinant human Nesfatin was intravenously administered on 3 days before the cell fusion.

Blood was collected from the tail vein of a mouse and incubated for 30 minutes at 37° C., centrifuged at 3000 rpm for 10 minutes and then the serum was recovered. Anti-Nesfatin antibody titers in the mouse serum were measured by ELISA. By using the obtained serum, the immunization was performed as in Example 9 (1) except for using an antibody manufactured by ZYMED as an alkaline phosphatase binding goat anti-mouse IgG antibody.

### (2) Preparation of Hybridoma by Cell Fusion

By using splenocytes of the mouse immunized in Example 11 (1), a hybridoma was prepared as in Example 9 (2).

### (3) Screening of Hybridoma

The obtained hybridoma was subject to the screening as in Example 9 (2). Further, a goat anti-mouse IgG-alkali phosphatase conjugate (Tago) diluted to 2000-fold with PBS containing 3% BSA and 0.2% skim milk was used instead of a goat anti-mouse IgG-alkali phosphatase conjugate diluted to 2000-fold with 3% BSA/PBS. The results are shown in Table 9. Each D, E, F and G in the Table represents a clone of hybridoma obtained from a mouse immunized by the following D, E, F and G, respectively. In addition, each numerical number in the Table represents the absorbance at 405 nm.

D: Nesfatin C-terminus peptide (NSF-C18)

E: Nesfatin N-terminus peptide (NSF-C19)

F: Nesfatin-1 C-terminus peptide (NSF1-C18)

G: Nesfatin-1 middle peptide (NSF1-C15)

TABLE 9

| | | Antigen | | | |
|---|---|---|---|---|---|
| Clone | No. | BSA | F-NAP | NAP-1 | Ig Type |
| D | 1 | 0.161 | 0.413 | 0.423 | |
| | 2 | 0.047 | 2.931 | 0.049 | IgG |
| | 3 | 0.179 | 1.150 | 0.883 | IgM |
| | 4 | 0.062 | 1.677 | 0.946 | IgM |
| | 5 | 0.039 | 3.309 | 0.049 | IgG |
| | 6 | 0.045 | 1.671 | 0.107 | IgM |
| | 7 | 0.371 | 1.873 | 1.005 | |
| | 8 | 0.041 | 0.943 | 0.048 | IgM |
| | 9 | 0.044 | 2.260 | 0.061 | IgM |
| | 10 | 0.264 | 1.384 | 0.771 | |
| | 11 | 0.043 | 1.652 | 0.326 | IgM |
| | 12 | 0.039 | 3.240 | 0.056 | IgG |
| | 13 | 0.061 | 1.471 | 1.037 | IgM |
| | 14 | 0.039 | 0.076 | 0.045 | |
| | 15 | 0.039 | 2.966 | 0.046 | IgG |
| | 16 | 0.448 | 0.937 | 1.196 | |
| | 17 | 0.081 | 0.786 | 0.285 | |
| | 18 | 0.042 | 1.301 | 0.055 | IgM |
| | 19 | 0.040 | 0.638 | 0.059 | |
| | 20 | 0.041 | 1.092 | 0.196 | |
| E | 1 | 0.034 | | 1.302 | IgG |
| | 2 | 0.032 | | 0.704 | IgM |
| | 3 | 0.03 | | 0.866 | IgG |
| E | 4 | 0.038 | 0.046 | 1.043 | IgM |
| | 5 | 0.110 | 1.450 | 1.152 | |
| | 6 | 0.038 | 0.082 | 0.193 | |
| | 7 | 0.257 | 0.596 | 0.504 | |
| F | 1 | 0.201 | 0.858 | 0.812 | |
| | 2 | 0.067 | 0.271 | 0.401 | |
| | 3 | 0.057 | 0.145 | 0.275 | |
| | 4 | 0.038 | 0.043 | 1.249 | IgG |
| | 5 | 0.292 | 0.658 | 0.924 | |
| | 6 | 0.293 | 0.703 | 1.192 | |
| G | 1 | 0.072 | 1.022 | 1.188 | IgM |
| | 2 | 0.057 | 0.718 | 0.327 | IgM |
| | 3 | 0.043 | 0.665 | 0.365 | IgM |
| | 4 | 0.090 | 0.460 | 0.511 | |
| | 5 | 0.039 | 0.592 | 0.055 | IgM |
| | 6 | 0.066 | 0.845 | 0.339 | |
| | 7 | 0.039 | 0.925 | 0.337 | IgM |
| | 8 | 0.120 | 0.681 | 0.655 | |
| | 9 | 0.044 | 1.112 | 0.081 | IgM |
| | 10 | 0.056 | 1.173 | 0.899 | IgM |
| | 11 | 0.116 | 0.810 | 0.598 | |
| | 12 | 0.052 | 0.911 | 0.493 | IgM |

### (4) Cloning of Hybridoma

The selected hybridoma was cloned as in Example 9 (4).

### (5) Culture of Hybridoma and Antibody Purification

Among 20 hybridoma clones producing anti-Nesfatin-1 antibodies obtained by the above procedures, 11 clones are cultured in large scale and the culture supernatant was recovered. Antibodies in the culture supernatant were purified by a protein G column according to an ordinary method.

### Example 13

### Reactivity Assessment of Mouse Monoclonal Antibody Against NESFATIN Partial Peptide

### (1) Reactivity Assessment of Antibody by Antigen ELISA

By using the purified IgG obtained in Example 12, reactivity assessment of each antibody was performed by antigen ELISA as in Example 5 (2). In addition, the reactivity assessment was conducted as in Example 5 (2) except for using a 96-well ELISA plate (MaxiSorp: Nunc) on which GST-fused recombinant of human Nesfatin-1, human Nesfatin, human NucB1-N77, rat Nesfatin or rat Nesfatin-1, diluted to 1 μg/mL with PBS (pH 7) was immobilized, as a subject for reactivity assessment. The results are shown in Tables 10 and 11. Each numerical number in the Table represents the absorbance at 405 nm. Further, clones shown in Tables are, for example, as follows, in the case of NAE-3, NAE-3 shows the same as that in E in Table 9: Clone No. 3 of a hybridoma obtained from a mouse immunized by Nesfatin N-terminus peptide (NSF-C19).

TABLE 10

| | Antigen | | | | |
|---|---|---|---|---|---|
| | Hu | Rat | Hu | Hu | Hu |
| Clone | NAP1 | F-NAP | F-NAP | NucB1-N77 | NucB1-full |
| NAE-3 | 3.399 | 0.084 | 2.666 | 0.07 | 0.06 |
| NAE-1 | 3.198 | 2.91 | 2.773 | 0.07 | 0.06 |
| NAD-15 | 0.11 | 3.137 | 3.19 | 0.07 | 0.06 |
| NAD-5 | 0.063 | 1.415 | 2.913 | 0.08 | 0.06 |
| NAD-12 | 0.079 | 0.1 | 3.211 | 0.08 | 0.06 |

TABLE 11

| | Antigen | | | | | |
|---|---|---|---|---|---|---|
| | Hu | Hu | Rat | Hu | Hu | |
| Clone | F-NAP | NAP-1 | NAP-1 | NucB1-N77 | F-NucB1 | Ig Type |
| NAF-7 | 0.09 | 1.75 | 0.08 | 0.06 | 0.03 | IgG |
| NAF-8 | 0.04 | 1.35 | 1.51 | 0.02 | 0.05 | IgM |
| NAF-9 | 0.04 | 1.39 | 1.16 | 0.02 | 0.04 | IgM |
| NAF-11 | 0.82 | 2.53 | 2.58 | 0.02 | 0.15 | IgG |
| NAF-12 | 0.71 | 1.86 | 1.42 | 0.28 | 0.07 | IgM |

As a result of Table 10, it was found that NAE1 showed a high reactivity to any of human Nesfatin, human Nesfatin-1 and rat Nesfatin, on the other hand, NAE3 showed a high reactivity to human Nesfatin and human Nesfatin-1, but did not show the cross-reactivity to a rat. Further, it was found that NAD15 showed a high reactivity to both human Nesfatin and rat Nesfatin, but did not show the reactivity to human Nesfatin-1. Any of the antibodies did not show the reactivity to NucB1-N77 at all.

As a result of Table 11, it was found that NAF11 showed a high reactivity to any of human Nesfatin, human Nesfatin-1 and rat Nesfatin-1, but did not show the cross-reactivity to human NucB1-N77.

NAE-1, NAF-7 and NAF-11 were respectively deposited at Depositary Authority: International Patent Organism Depositary, National Institute of Advanced Industrial Science and Technology (Accession number: FERM ABP-10881, ABP-10882 and ABP-10883; Date of the acceptance: Jul. 27, 2007).

### Example 14

### Construction of Nesfatin-1 Sandwitch ELISA Using Mouse Monoclonal Antibody (NAE1) Against NESFATIN Partial Peptide

### (1) Construction of Sandwitch ELISA

### (A) Preparation of Biotinylated Antibody

Biotinylation of an anti-Nesfatin-1 C-terminus peptide mouse monoclonal antibody (NAE1 or NAE3) (IgG) obtained in Example 12 was performed as in Example 7 (1) (A).

### (B) Preparation of Antibody-Immobilized Plate

An antibody-immobilized plate on which antibody No. 4998 was immobilized was prepared as in Example 7 (1) (B) by using an antibody (antigen affinity-purified anti-Nesfatin-1 C-terminus peptide rabbit polyclonal antibody (antibody No. 4998)) prepared in Example 5.

### (C) Construction of Sandwitch ELISA

A sandwitch ELISA using an anti-Nesfatin-1 C-terminus peptide PAb (antibody No. 4998)-immobilized plate prepared in the above-mentioned Example 14 (1) (B) and a biotinylated IgG (biotinylated NAE1) prepared in the above-mentioned Example 14 (1) (A) was examined.

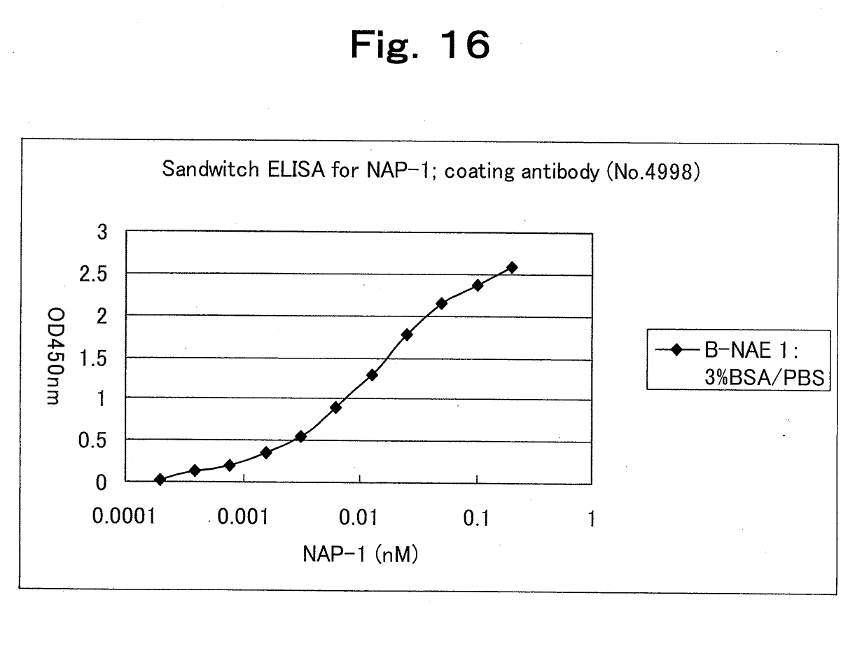

As a test substance, a test substance dilution series of 3% BSA-containing 10 mM PBS (pH 7.2) solution containing purified recombinant human Nesfatin-1 (standard substance) in the range of 0 to 200 μM was prepared and used. As for a biotinylated antibody, 3% BSA-containing 10 mM PBS (pH 7.2) solution containing 2 μg/mL of biotinylated antibody was used. The rest was performed as in Example 7 (1) (C). As for a test substance at each concentration of 0 to 200 μM, an absorption intensity of this solution was measured at the wavelength of 450 nm, and was blotted. The results are shown in FIG. 16.

### (2) Assessment of the Sensitivity for Species Difference in a Sandwitch ELISA

By using an anti-Nesfatin-1 C-terminus peptide PAb (antibody No. 4998)-immobilized plate prepared in the above-mentioned Example 14 (1) (B) and a biotinylated IgG (biotinylated NAE1 or biotinylated NAE3) prepared in the above-mentioned Example 14 (1) (A), the cross-reactivity to rat Nesfatin-1 was examined.

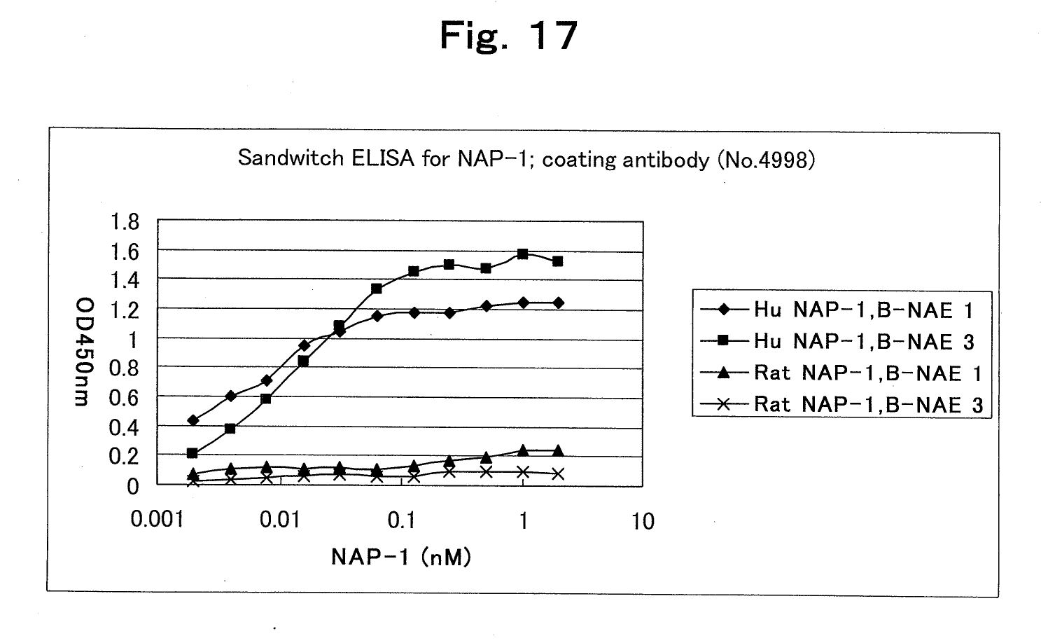

As for a test substance, a test substance dilution series of 3% BSA-containing 10 mM PBS (pH 7.2) solution containing purified recombinant human Nesfatin-1 (standard substance) or recombinant rat Nesfatin-1 (standard substance) in the range of 0 to 2 nM was prepared and used. As for a biotinylated IgG, 3% BSA-containing PBS (pH 7.2) solution containing 2 μg/mL of biotinylated IgG was used. The rest was performed as in Example 7 (1) (C). As for a test substance at each concentration of 0 to 2 nM, an absorption intensity of this solution was measured at the wavelength of 450 nm, and was blotted. The results are shown in FIG. 17.

As a result, it was found that both the biotinylated

NAE1 and the biotinylated NAE3 detected only human Nesfatin-1 and did not show the cross-reactivity to rat Nesfatin-1.

### (3) Assessment of NucB1-N77 Cross-Reactivity

By using an anti-Nesfatin-1 C-terminus peptide PAb (antibody No. 4998)-immobilized plate prepared in the above-mentioned Example 14 (1) (B) and a biotinylated IgG (biotinylated NAE1) prepared in the above-mentioned Example 14 (1) (A), the cross-reactivity to NucB1-N77 was examined.

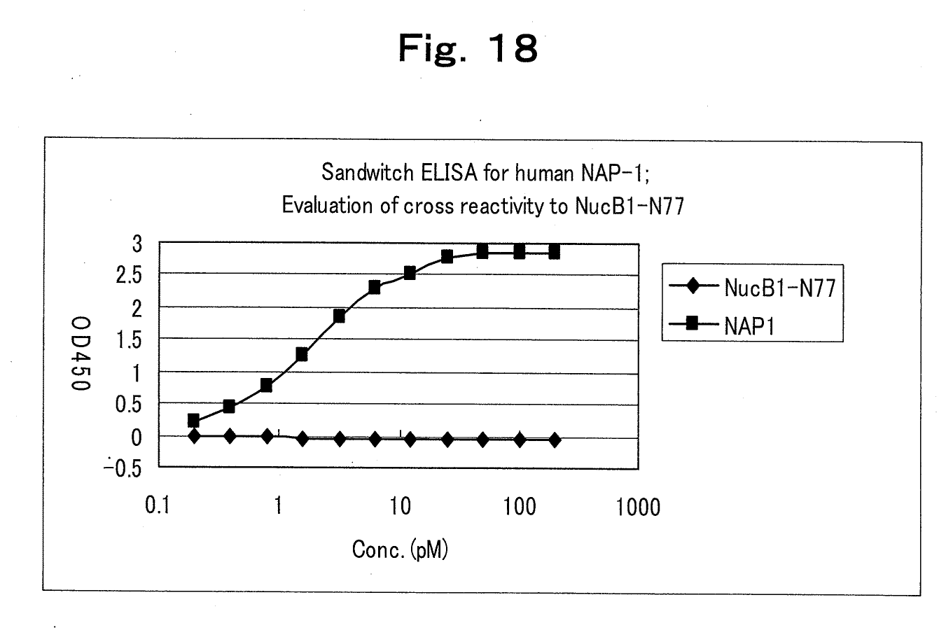

As a test substance, a test substance dilution series of 3% BSA-containing 10 mM PBS (pH 7.2) solution containing purified recombinant human Nesfatin-1 (standard substance) or purified recombinant human NucB1-N77 in the range of 0 to 200 μM was prepared and used. As for a biotinylated IgG, 3% BSA-containing PBS (pH 7.2) solution containing 2 μg/mL of biotinylated IgG was used. The rest was performed as in Example 7 (1) (C). As for a test substance at each concentration of 0 to 2 nM, an absorption intensity of this solution was measured at the wavelength of 450 nm, and was blotted. The results are shown in FIG. 18.

As a result, it was found that the present sandwitch ELISA detected only human Nesfatin-1 and did not show the cross-reactivity to human NucB1-N77.

### (4) Assessment of Nesfatin Cross-Reactivity

By using an anti-Nesfatin-1 C-terminus peptide PAb (antibody No. 4998)-immobilized plate prepared in the above-mentioned Example 14 (1) (B) and a biotinylated IgG (biotinylated NAE1) prepared in the above-mentioned Example 14 (1) (A), the cross-reactivity to purified recombinant human Nesfatin was examined.

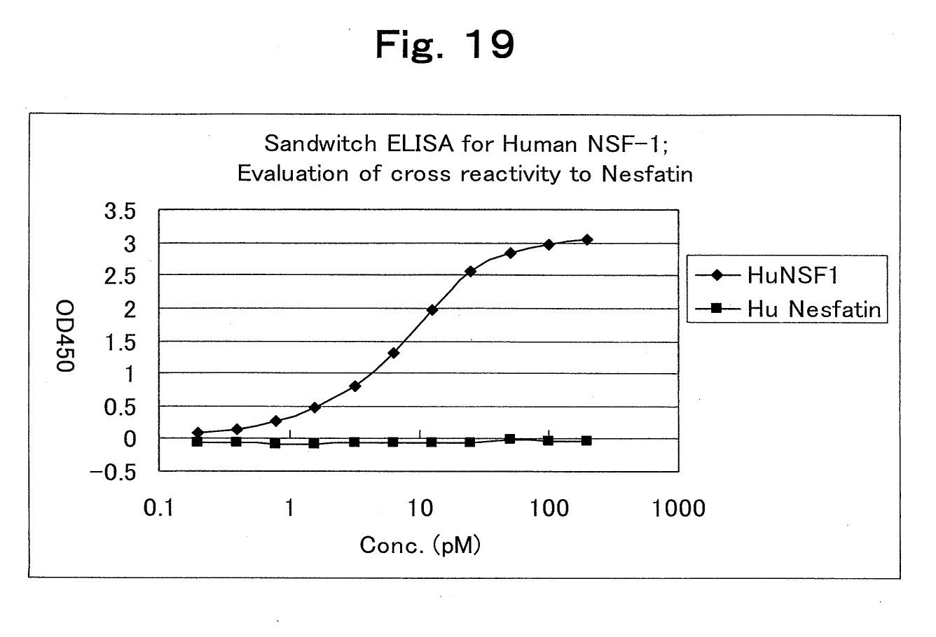

As a test substance, a test substance dilution series of 3% BSA-containing 10 mM PBS (pH 7.2) solution containing purified recombinant human Nesfatin-1 (standard substance) or recombinant human Nesfatin in the range of 0 to 200 μM was prepared and used. As for a biotinylated IgG, 3% BSA-containing PBS (pH 7.2) solution containing 2 μg/mL of biotinylated IgG was used. The rest was performed as in Example 7 (1) (C). As for a test substance at each concentration of 0 to 200 pM, an absorption intensity of this solution was measured at the wavelength of 450 nm, and was blotted. The results are shown in FIG. 19.

As a result, it was found that the present sandwitch

ELISA detected only human Nesfatin-1 and did not show the cross-reactivity to human Nesfatin.

### Example 15

### Construction of Nesfatin Sandwitch ELISA Using Mouse Monoclonal Antibody (NAE1) Against NESFATIN Partial Peptide

### (1) Construction of Sandwitch ELISA

### (A) Preparation of Biotinylated Antibody

Biotinylation of an anti-Nesfatin-1 C-terminus peptide mouse monoclonal antibody (NAE1, NAE3 or NAD15) (IgG) obtained in Example 12 was performed as in Example (1) (A).

### (B) Preparation of Antibody-Immobilized Plate

By using an antibody (antigen peptide affinity-purified anti-Nesfatin C-terminus peptide PAb (antibody No. 4994) or an anti-Nesfatin-1 C-terminus peptide mouse monoclonal antibody (NAD15)), an antibody-immobilized plate on which antibody No. 4994 or NAD15 was immobilized was prepared as in Example 7 (1) (B).

### (C) Construction of Sandwitch ELISA

By using an antibody No. 4994-immobilized plate and a biotinylated NAE1, NAE3 or NAD15, the reactivity to human Nesfatin was examined.

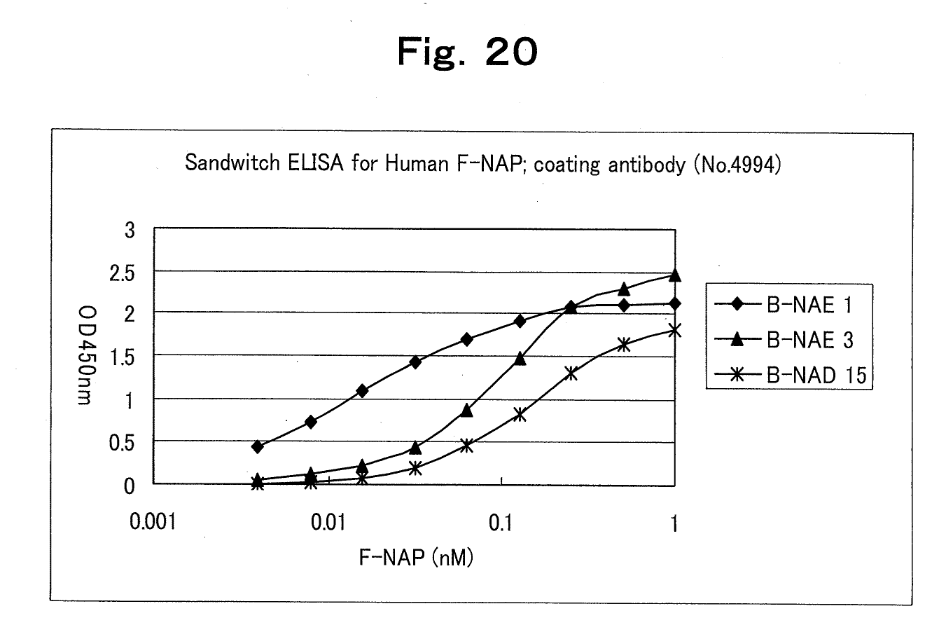

As a test substance, a test substance dilution series of 3% BSA-containing 10 mM PBS (pH 7.2) solution containing purified recombinant Nesfatin (standard substance) in the range of 0 to 1 nM was prepared and used. As for a biotinylated IgG, 3% BSA-containing PBS (pH 7.2) solution containing 2 μg/mL of biotinylated IgG was used. The rest was performed as in Example 7 (1) (C). As for a test substance at each concentration of 0 to 1 nM, an absorption intensity of this solution was measured at the wavelength of 450 nm, and was blotted. The results are shown in FIG. 20.

### (2) Reactivity Assessment to Species Difference of Biotinylated Antibody

By using an antibody NAD15 (an mouse monoclonal antibody against a human Nesfatin C-terminus peptide)-immobilized plate and a biotinylated NAE1 or NAE3, the cross-reactivity with recombinant human Nesfatin or recombinant rat Nesfatin was examined.

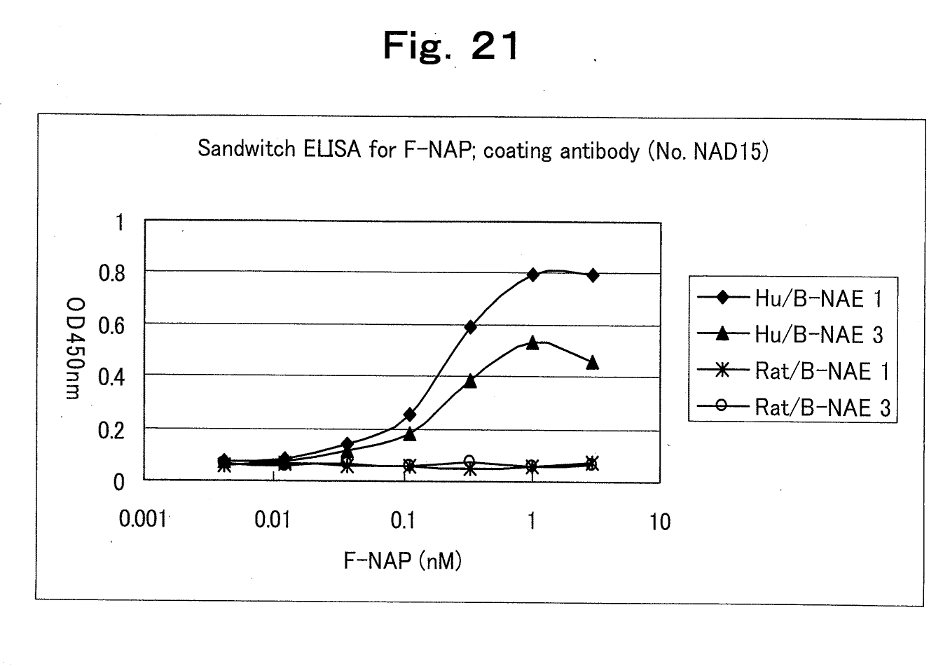

As a test substance, a test substance dilution series of 3% BSA-containing 10 mM PBS (pH 7.2) solution containing purified recombinant human Nesfatin (standard substance) or rat Nesfatin in the range of 0 to 10 nM was prepared and used. As for a biotinylated IgG, 3% BSA-containing PBS (pH 7.2) solution containing 2 μg/mL of biotinylated IgG was used. The rest was performed as in Example 7 (1) (C). As for a test substance at each concentration of 0 to 10 nM, an absorption intensity of this solution was measured at the wavelength of 450 nm, and was blotted. The results are shown in FIG. 21.

As a result, it was found that both NAE1 and NAE3 did not have the reactivity to rat Nesfatin.

### (3) Assessment of Cross-Reactivity to NucB1

As a result of Example 15 (1), it was shown that the sandwitch ELISA using an antibody No. 4994-immobilized plate and a biotinylated NAE1 (detection antibody) detected human Nesfatin with the highest sensitivity. Therefore, as for the measurement system, the cross-reactivity with human NucB1 was examined.

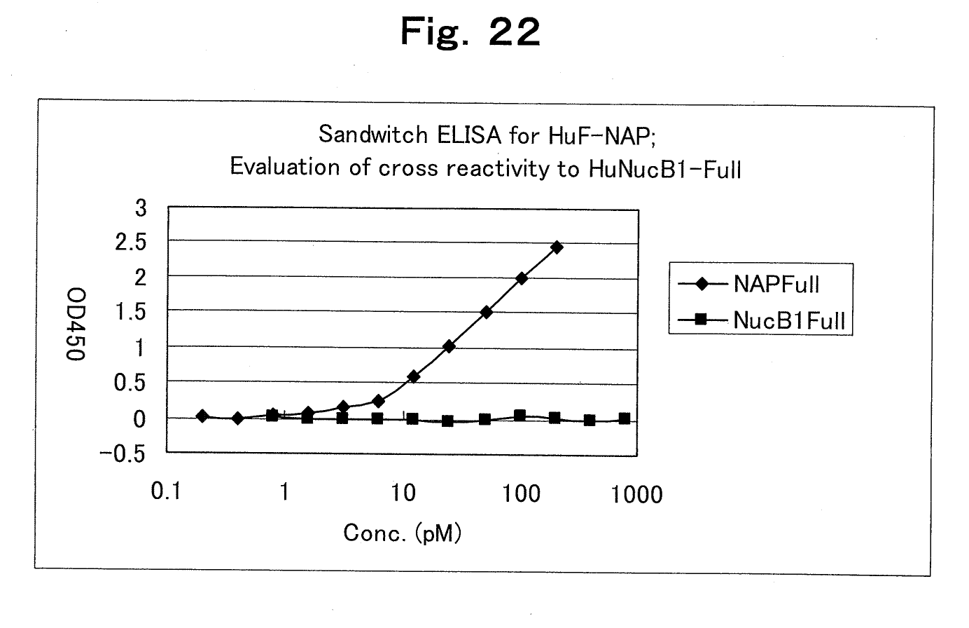

As a test substance, a test substance dilution series of 3% BSA-containing 10 mM PBS (pH 7.2) solution containing purified recombinant human NucB1 (GST fusion protein) in the range of 0 to 800 pM and a test substance dilution series of 3% BSA-containing 10 mM PBS (pH 7.2) solution containing purified recombinant human Nesfatin in the range of 0 to 200 pM were prepared and used. As for a biotinylated IgG, 3% BSA-containing PBS (pH 7.2) solution containing 2 μg/mL of biotinylated IgG was used. The rest was performed as in Example 7 (1) (C). As for a test substance at each concentration of 0 to 1 nM, an absorption intensity of this solution was measured at the wavelength of 450 nm, and was blotted. The results are shown in FIG. 22.

As a result, the cross-reactivity to NucB1 was not observed at all. Therefore, the present measurement system is considered to be a Nesfatin-specific sandwitch ELISA.

### Example 16

### Construction of Nesfatin-1 Sandwitch ELISA Using Mouse Monoclonal Antibody (NAF11) Against NESFATIN Partial Peptide

### (1) Construction of Sandwitch ELISA

### (A) Preparation of Biotinylated Antibody

Among the mouse monoclonal antibodies obtained in Example 12, by using an anti-Nesfatin-1 C-terminus peptide mouse monoclonal antibody (NAE11) reacting with human and rat Nesfatin-1 and not showing the cross-reactivity with human NucB1, biotinylation of the IgG was performed as in Example 7 (1) (A).

### (B) Preparation of Antibody-Immobilized Plate

By using an antibody (antigen peptide affinity-purified anti-Nesfatin-1 C-terminus peptide PAb (antibody No. 4998)), an antibody-immobilized plate on which antibody No. 4998 was immobilized was prepared as in Example 7 (1) (B).

### (C) Construction of Sandwitch ELISA

A sandwitch ELISA using an anti-Nesfatin-1 C-terminus peptide PAb (antibody No. 4998)-immobilized plate prepared in the above-mentioned Example 16 (1) (B) and a biotinylated IgG (NAF11) prepared in the above-mentioned Example 16 (1) (A) was examined.

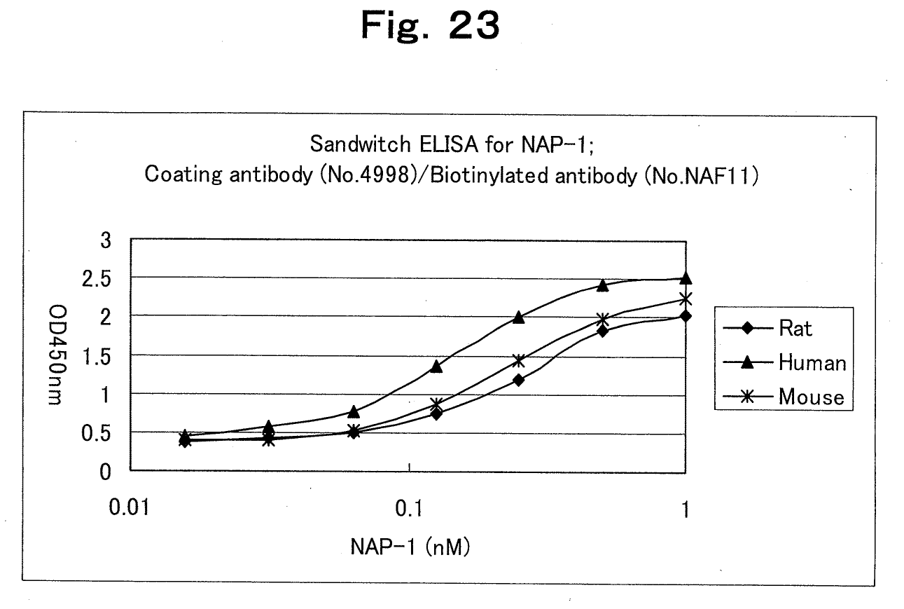

As a test substance, a test substance dilution series of 3% BSA-containing 10 mM PBS (pH 7.2) solution containing purified recombinant Nesfatin-1 (standard substance) in the range of 0 to 1 nM was prepared and used. As for a biotinylated IgG, 3% BSA-containing PBS (pH 7.2) solution containing 2 μg/mL of biotinylated IgG was used. The rest was performed as in Example 7 (1) (C). As for a test substance at each concentration of 0 to 1 nM, an absorption intensity of this solution was measured at the wavelength of 450 nm, and was blotted. The results are shown in FIG. 23.

### Example 17

### Construction of Nesfatin-1 Sandwitch ELISA Using Mouse Monoclonal Antibody (NAF11) Against NESFATIN Partial Peptide

### (1) Construction of Sandwitch ELISA

### (A) Preparation of Biotinylated Antibody

Among the mouse monoclonal antibodies obtained in Example 12, by using an anti-Nesfatin-1 C-terminus peptide mouse monoclonal antibody (NAE11) reacting with human and rat Nesfatin-1 and not showing the cross-reactivity to human NucB1, biotinylation of IgG was performed as in Example 7 (1) (A).

### (B) Preparation of Antibody-Immobilized Plate

By using an antibody (antigen peptide affinity-purified anti-Nesfatin C-terminus peptide PAb (antibody No. 4994)), an antibody-immobilized plate on which antibody No. 4994 was immobilized was prepared as in Example 7 (1) (B).

### (C) Construction of Sandwitch ELISA

By using an anti-Nesfatin-1 C-terminus peptide PAb (antibody No. 4994)-immobilized plate prepared in the above-mentioned Example 17 (1) (B) and a biotinylated antibody (antibody NAF11), the cross-reactivity to human, mouse or rat was examined.

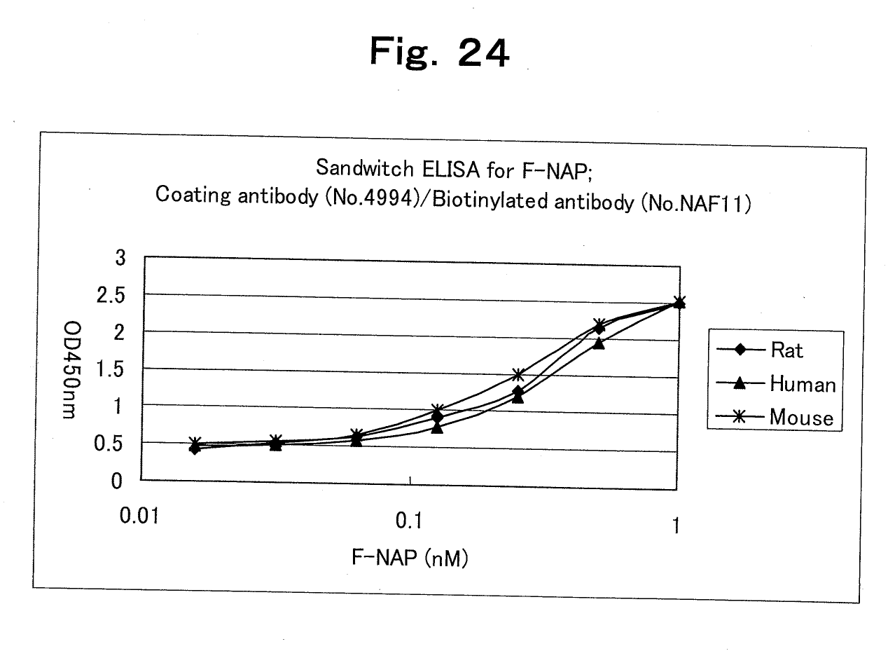

As a test substance, a test substance dilution series of 3% BSA-containing 10 mM PBS (pH 7.2) solution containing purified recombinant human, rat or mouse Nesfatin (standard substance) in the range of 0 to 1 nM was prepared and used. As for a biotinylated IgG, 3% BSA-containing PBS (pH 7.2) solution containing 2 μg/mL of biotinylated IgG was used. The rest was performed as in Example 7 (1) (C). As for a test substance at each concentration of 0 to 1 nM, an absorption intensity of this solution was measured at the wavelength of 450 nm, and was blotted. The results are shown in FIG. 24.

### INDUSTRIAL APPLICABILITY

According to the present invention, an antibody performing an antigen-antibody reaction with Nesfatin-1, but not substantially performing the antigen-antibody reaction with Nesfatin or NucB1, and an antibody performing an antigen-antibody reaction with Nesfatin-1, but not substantially performing the antigen-antibody reaction with Nesfatin and NucB1, and further, an immunological detection method using these antibodies and detection kit of Nesfatin-1 comprising these antibodies, are provided.

Furthermore, according to the present invention, an antibody performing an antigen-antibody reaction with Nesfatin, but not substantially performing the antigen-antibody reaction with NucB1, and further, an immunological detection method using the antibody and detection kit of Nesfatin comprising the antibody, are provided.

### EXPLANATION OF SEQUENCE LISTINGS

(1) SEQ ID NO: 1: an amino acid sequence of NSF1-C18
(2) SEQ ID NO: 2: an amino acid sequence of NSF1-M15
(3) SEQ ID NO: 3: an amino acid sequence of NSF-C18
(4) SEQ ID NO: 4: an amino acid sequence of NSF-N19
(5) SEQ ID NO: 5: an amino acid sequence of human-derived Nesfatin-1
(6) SEQ ID NO: 6: an amino acid sequence of mouse-derived Nesfatin-1
(7) SEQ ID NO: 7: an amino acid sequence of rat-derived Nesfatin-1
(8) SEQ ID NO: 8: an amino acid sequence of human-derived NESFATIN
(9) SEQ ID NO: 9: an amino acid sequence of human-derived NESFATIN (Mature)
(10) SEQ ID NO: 10: an amino acid sequence of mouse-derived NESFATIN
(11) SEQ ID NO: 11: an amino acid sequence of mouse-derived NESFATIN (Mature)
(12) SEQ ID NO: 12: an amino acid sequence of rat-derived NESFATIN
(13) SEQ ID NO: 13: an amino acid sequence of rat-derived NESFATIN (Mature)
(14) SEQ ID NO: 14: an amino acid sequence of human-derived NucB1 (Mature)
(15) SEQ ID NO: 15: an amino acid sequence of mouse-derived NucB1 (Mature)
(16) SEQ ID NO: 16: an amino acid sequence of rat-derived NucB1 (Mature)
(17) SEQ ID NO: 17: an amino acid sequence of human-derived NucB1-N77
(18) SEQ ID NO: 18: an amino acid sequence of mouse-derived NucB1-N77
(19) SEQ ID NO: 19: an amino acid sequence of rat-derived NucB1-N77
(20) SEQ ID NO: 20: Human NESFATIN and Human NESFATIN-1 1st PCR Primer hNucB2-F0191
(21) SEQ ID NO: 21: Human NESFATIN and Human NESFATIN-1 1st PCR Primer hNucB2-R1549
(22) SEQ ID NO: 22: Human NESFATIN and Human NESFATIN-1 2nd PCR Primer hNucB2-F292
(23) SEQ ID NO: 23: Human NESFATIN-1 2nd PCR Primer hNucB2-R514
(24) SEQ ID NO: 24: Human NESFATIN 2nd PCR Primer hNucB2 R1461
(25) SEQ ID NO: 25: Human NucB1 and Human NucB1-N77 1st PCR Primer hNucB1-F061
(26) SEQ ID NO: 26: Human NucB1 and Human NucB1-N77 1st PCR Primer hNucB1-R1376
(27) SEQ ID NO: 27: Human NucB1 and Human NucB1-N77 2nd PCR Primer hNucB1-F096
(28) SEQ ID NO: 28: Human NucB1-N77 2nd PCR Primer hNucB1-R303
(29) SEQ ID NO: 29: a thrombin recognition site
(30) SEQ ID NO: 30: mouse NESFATIN and mouse NESFATIN-1 1st PCR Primer mNucB2-F337
(31) SEQ ID NO: 31: mouse NESFATIN and mouse NESFATIN-1 1st PCR Primer mNucB2-R1613
(32) SEQ ID NO: 32: mouse NESFATIN and mouse NESFATIN-1 2nd PCR Primer mNucB2-F360
(33) SEQ ID NO: 33: mouse NESFATIN-1 2nd PCR Primer mNucB2-R582
(34) SEQ ID NO: 34: mouse NESFATIN 2nd and 3rd PCR Primer mNucB2-R1527
(35) SEQ ID NO: 35: mouse NESFATIN 3rd PCR Primer His-Thr-For
(36) SEQ ID NO: 36: mouse NucB1 and mouse NucB1-N77 1st PCR Primer mNucB1-F009
(37) SEQ ID NO: 37: mouse NucB1 and mouse NucB1-N77 1st PCR Primer mNucB1-R1406
(38) SEQ ID NO: 38: mouse NucB1 and mouse NucB1-N77 2nd PCR Primer mNucB1-F094
(39) SEQ ID NO: 39: mouse NucB1-N77 2nd PCR Primer mNucB1-R301
(40) SEQ ID NO: 40: mouse NucB1 2nd PCR Primer mNucB1-R1360
(41) SEQ ID NO: 41: rat NESFATIN and rat NESFATIN-1 1st PCR Primer rNucB2-F204
(42) SEQ ID NO: 42: rat NESFATIN and rat NESFATIN-1 1st PCR Primer rNucB2-R1540
(43) SEQ ID NO: 43: rat NESFATIN and rat NESFATIN-1 2nd PCR Primer ratNucB2-F286
(44) SEQ ID NO: 44: rat NESFATIN-1 2nd PCR Primer ratNucB2-R507
(45) SEQ ID NO: 45: rat NESFATIN 2nd and 3rd PCR Primer ratNucB2-R1531
(46) SEQ ID NO: 46: rat NucB1 and rat NucB1-N77 1st PCR Primer ratNucB1-F001
(47) SEQ ID NO: 47: rat NucB1 and rat NucB1-N77 1st PCR Primer ratNucB1-R1402
(48) SEQ ID NO: 48: rat NucB1 and rat NucB1-N77 2nd PCR Primer rNucB1-F078
(49) SEQ ID NO: 49: rat NucB1-N77 2nd PCR Primer rNucB1-R285
(50) SEQ ID NO: 50: rat NucB1 2nd PCR Primer rNucB1-R1357

1. An antibody performing an antigen-antibody reaction with Nesfatin-1, but not substantially performing the antigen-antibody reaction with Nesfatin.

2. The antibody according to claim 1, obtainable by using a peptide having the following amino acid sequence as an immunogen
NSF1-C18:
(SEQ ID NO: 1)
-Gly-Cys-Ser-Lys-Glu-Leu-Asp-Leu-Val-Ser-His-
His-Val-Arg-Thr-Lys-Leu-Asp-Glu-Leu.

3. An antibody performing an antigen-antibody reaction with Nesfatin-1, but not substantially performing the antigen-antibody reaction with NucB1.

4. The antibody according to claim 3, wherein the antibody is a monoclonal antibody and is produced by a hybridoma deposited under an accession number: FERM ABP-10881, FERM ABP-10882, FERM ABP-10883 or FERM ABP-10884.

5. An antibody performing an antigen-antibody reaction with Nesfatin-1, but not substantially performing the antigen-antibody reaction with Nesfatin and NucB1.

6. The antibody according to claim 5, wherein the antibody is a monoclonal antibody and is produced by a hybridoma deposited under an accession number: FERM ABP-10882 or FERM ABP-10884.

7. An antibody performing an antigen-antibody reaction with Nesfatin, but not substantially performing the antigen-antibody reaction with Nesfatin-1 and NucB1.

8. A hybridoma deposited under an accession number: FERM ABP-10881, FERM ABP-10882, FERM ABP-10883 or FERM ABP-10884.

9. An immunological detection method of Nesfatin-1 comprising:
a step of bringing a test substance into contact with an antibody selected from the group consisting of:
(1) an antibody performing an antigen-antibody reaction with Nesfatin-1, but not substantially performing the antigen-antibody reaction with Nesfatin,
(2) an antibody according to (1), obtainable by using a peptide having the following amino acid sequence as an immunogen:
NSF1-C18:
(SEQ ID NO: 1)
-Gly-Cys-Ser-Lys-Glu-Leu-Asp-Leu-Val-Ser-His-
His-Val-Arg-Thr-Lys-Leu-Asp-Glu-Leu,
(3) an antibody performing an antigen-antibody reaction with Nesfatin-1, but not substantially performing the antigen-antibody reaction with NucB1,
(4) a monoclonal antibody produced by a hybridoma deposited under an accession number: FERM ABP-10881, FERM ABP-10882, FERM ABP-10883 or FERM ABP-10884
(5) an antibody performing an antigen-antibody reaction with Nesfatin-1, but not substantially performing the antigen-antibody reaction with Nesfatin and NucB1, and
(6) a monoclonal antibody produced by a hybridoma deposited under an accession number: FERM ABP-10882 or FERM ABP-10884; and
a step of detecting an antibody performing an antigen-antibody reaction with the test substance.

10. An immunological detection method of Nesfatin comprising:
a step of bringing a test substance into contact with an antibody performing an antigen-antibody reaction with Nesfatin, but not substantially performing the antigen-antibody reaction with Nesfatin-1 and NucB1; and
a step of detecting an antibody performing an antigen-antibody reaction with the test substance.

11. An immunological detection method of Nesfatin-1 in a test substance, using two types of antibodies selected from the group consisting of:
(1) an antibody performing an antigen-antibody reaction with Nesfatin-1, but not substantially performing the antigen-antibody reaction with Nesfatin,
(2) an antibody according to (1), obtainable by using a peptide having the following amino acid sequence as an immunogen:
NSF1-C18:
(SEQ ID NO: 1)
-Gly-Cys-Ser-Lys-Glu-Leu-Asp-Leu-Val-Ser-His-
His-Val-Arg-Thr-Lys-Leu-Asp-Glu-Leu,
(3) an antibody performing an antigen-antibody reaction with Nesfatin-1, but not substantially performing the antigen-antibody reaction with NucB1,
(4) a monoclonal antibody produced by a hybridoma deposited under an accession number: FERM ABP-10881, FERM ABP-10882, FERM ABP-10883 or FERM ABP-10884
(5) an antibody performing an antigen-antibody reaction with Nesfatin-1, but not substantially performing the antigen-antibody reaction with Nesfatin and NucB1, and
(6) a monoclonal antibody produced by a hybridoma deposited under an accession number: FERM ABP-10882 or FERM ABP-10884.

12. The immunological detection method according to claim 11, comprising:
a step of bringing a test substance into contact with a primary antibody immobilized on a solid phase;
a step of bringing the test substance to which the primary antibody is bound into contact with a labeled secondary antibody; and then
a step of detecting a secondary antibody performing an antigen-antibody reaction with the test substance; and
wherein one of the primary antibody and the secondary antibody is an antibody selected from the group consisting of
(1) an antibody performing an antigen-antibody reaction with Nesfatin-1, but not substantially performing the antigen-antibody reaction with Nesfatin,
(2) an antibody according to (1), obtainable by using a peptide having the following amino acid sequence as an immunogen:
NSF1-C18:
(SEQ ID NO: 1)
-Gly-Cys-Ser-Lys-Glu-Leu-Asp-Leu-Val-Ser-His-
His-Val-Arg-Thr-Lys-Leu-Asp-Glu-Leu,
(3) an antibody performing an antigen-antibody reaction with Nesfatin-1, but not substantially performing the antigen-antibody reaction with Nesfatin and NucB1, and
(4) a monoclonal antibody produced by a hybridoma deposited under an accession number: FERM ABP-10882 or FERM ABP-10884; and
wherein the other of the primary antibody and the secondary antibody is an antibody selected from the group consisting of:
(1) an antibody performing an antigen-antibody reaction with Nesfatin-1, but not substantially performing the antigen-antibody reaction with NucB1,
(2) a monoclonal antibody produced by a hybridoma deposited under an accession number: FERM ABP-10881, FERM ABP-10882, FERM ABP-10883 or FERM ABP-10884
(3) an antibody performing an antigen-antibody reaction with Nesfatin-1, but not substantially performing the antigen-antibody reaction with Nesfatin and NucB1, and
(4) a monoclonal antibody produced by a hybridoma deposited under an accession number: FERM ABP-10882 or FERM ABP-10884.

13. The immunological detection method according to claim 12, wherein
the primary antibody is
(1) an antibody performing an antigen-antibody reaction with Nesfatin-1, but not substantially performing the antigen-antibody reaction with Nesfatin, or
(2) an antibody according to (1), obtainable by using a peptide having the following amino acid sequence as an immunogen: NSF1-C18: -Gly-Cys-Ser-Lys-Glu-Leu-Asp-Leu-Val-Ser-His-His-Val-Arg-Thr-Lys-Leu-Asp-Glu-Leu (SEQ ID NO:1), and
the secondary antibody is an antibody produced by a hybridoma deposited under an accession number: FERM ABP-10881 or FERM ABP-10883.

14. The immunological detection method according to claim 12, wherein
the primary antibody is an antibody produced by a hybridoma deposited under an accession number: FERM ABP-10884 or FERM ABP-10882, and
the secondary antibody is
(1) an antibody performing an antigen-antibody reaction with Nesfatin-1, but not substantially performing the antigen-antibody reaction with Nesfatin, or
(2) an antibody according to (1), obtainable by using a peptide having the following amino acid sequence as an immunogen: NSF1-C18: -Gly-Cys-Ser-Lys-Glu-Leu-Asp-Leu-Val-Ser-His-His-Val-Arg-Thr-Lys-Leu-Asp-Glu-Leu (SEQ ID NO:1).

15. The immunological detection method according to claim 11, having a sensitivity discriminating between a test substance at a Nesfatin-1 concentration of less than 30 pM and a control substance at a Nesfatin-1 concentration of 0 pM.

16. An immunological detection method of Nesfatin in a test substance, comprising:
a step of bringing a test substance into contact with a primary antibody immobilized on a solid phase;
a step of bringing the test substance to which the primary antibody is bound into contact with a labeled secondary antibody; and then a step of detecting a secondary antibody performing an antigen-antibody reaction with the test substance; and
wherein one of the primary antibody and the secondary antibody is an antibody performing an antigen-antibody reaction with Nesfatin, but not substantially performing the antigen-antibody reaction with Nesfatin-1 and NucB1; and
wherein the other of the primary antibody and the secondary antibody is an antibody performing an antigen-antibody reaction with Nesfatin, but not substantially performing the antigen-antibody reaction with NucB1.

17. The immunological detection method according to claim 16, having a sensitivity discriminating between a test substance at a Nesfatin concentration of less than 30 pM and a control substance at a Nesfatin concentration of 0 pM.

18. The immunological detection method according to claim 16, wherein
the primary antibody is an antibody performing an antigen-antibody reaction with Nesfatin, but not substantially performing the antigen-antibody reaction with Nesfatin-1 and NucB1, and
the secondary antibody is an antibody produced by a hybridoma deposited under an accession number: FERM ABP-10881 or FERM ABP-10883.

19. A detection kit of Nesfatin-1 used for the detection method according to claim 12, comprising:
a solid phase on which a primary antibody is immobilized, and a labeled secondary antibody; and
wherein one of the primary antibody and the secondary antibody is an antibody selected from the group consisting of
(1) an antibody performing an antigen-antibody reaction with Nesfatin-1, but not substantially performing the antigen-antibody reaction with Nesfatin,
(2) an antibody according to (1), obtainable by using a peptide having the following amino acid sequence as an immunogen:
NSF1-C18:
(SEQ ID NO: 1)
-Gly-Cys-Ser-Lys-Glu-Leu-Asp-Leu-Val-Ser-His-
His-Val-Arg-Thr-Lys-Leu-Asp-Glu-Leu,
(3) an antibody performing an antigen-antibody reaction with Nesfatin-1, but not substantially performing the antigen-antibody reaction with Nesfatin and NucB1, and
(4) a monoclonal antibody produced by a hybridoma deposited under an accession number: FERM ABP-10882 or FERM ABP-10884 and
wherein the other of the primary antibody and the secondary antibody is an antibody selected from the group consisting of:
(1) an antibody performing an antigen-antibody reaction with Nesfatin-1, but not substantially performing the antigen-antibody reaction with NucB1,
(2) a monoclonal antibody produced by a hybridoma deposited under an accession number: FERM ABP-10881, FERM ABP-10882, FERM ABP-10883 or FERM ABP-10884
(3) an antibody performing an antigen-antibody reaction with Nesfatin-1, but not substantially performing the antigen-antibody reaction with Nesfatin and NucB1, and
(4) a monoclonal antibody produced by a hybridoma deposited under an accession number: FERM ABP-10882 or FERM ABP-10884.

20. A detection kit of Nesfatin used for the detection method according to claim 16, comprising:
a solid phase on which a primary antibody immobilized, and a labeled secondary antibody; and
wherein one of the primary antibody and the secondary antibody is an antibody performing an antigen-antibody reaction with Nesfatin, but not substantially performing the antigen-antibody reaction with Nesfatin-1 and NucB1; and
wherein the other of the primary antibody and the secondary antibody being an antibody performing an antigen-antibody reaction with Nesfatin, but not substantially performing the antigen-antibody reaction with NucB1.

21. An immunological detection method of Nesfatin-1, comprising:
a step of mixing a labeled Nesfatin-1 standard substance with a test substance;
a step of bringing an antibody selected from the group consisting of:
(1) an antibody performing an antigen-antibody reaction with Nesfatin-1, but not substantially performing the antigen-antibody reaction with Nesfatin,
(2) an antibody according to (1), obtainable by using a peptide having the following amino acid sequence as an immunogen:
NSF1-C18:
(SEQ ID NO: 1)
-Gly-Cys-Ser-Lys-Glu-Leu-Asp-Leu-Val-Ser-His-
His-Val-Arg-Thr-Lys-Leu-Asp-Glu-Leu,
(3) an antibody performing an antigen-antibody reaction with Nesfatin-1, but not substantially performing the antigen-antibody reaction with NucB1,
(4) a monoclonal antibody produced by a hybridoma deposited under an accession number: FERM ABP-10881, FERM ABP-10882, FERM ABP-10883 or FERM ABP-10884
(5) an antibody performing an antigen-antibody reaction with Nesfatin-1, but not substantially performing the antigen-antibody reaction with Nesfatin and NucB1, and
(6) a monoclonal antibody produced by a hybridoma deposited under an accession number: FERM ABP-10882 or FERM ABP-10884, into contact with the test substance mixed with the labeled Nesfatin-1 standard substance; and
a step of detecting a labeled Nesfatin-1 standard substance performing an antigen-antibody reaction with the antibody.

22. The immunological detection method according to claim 21, wherein the antibody is: (1) the antibody performing an antigen-antibody reaction with Nesfatin-1, but not substantially performing the antigen-antibody reaction with Nesfatin and NucB1, or
(2) the monoclonal antibody produced by a hybridoma deposited under an accession number: FERM ABP-10882 or FERM ABP-10884.

23. An immunological detection method of Nesfatin, comprising:
a step of mixing a labeled Nesfatin standard substance with a test substance;
a step of bringing an antibody performing an antigen-antibody reaction with Nesfatin, but not substantially performing the antigen-antibody reaction with NucB1 into contact with the test substance mixed with the labeled Nesfatin standard substance; and
a step of detecting a labeled Nesfatin standard substance performing an antigen-antibody reaction with the antibody.

24. The immunological detection method according to claim 23, wherein the antibody is an antibody performing an antigen-antibody reaction with Nesfatin, but not substantially performing the antigen-antibody reaction with Nesfatin-1 and NucB1.

25. A detection kit of Nesfatin-1 used for the detection method according to claim 21, comprising:
a labeled Nesfatin-1 standard substance and an antibody performing an antigen-antibody reaction with Nesfatin-1; and
wherein the antibody is an antibody selected from the group consisting of:
(1) an antibody performing an antigen-antibody reaction with Nesfatin-1, but not substantially performing the antigen-antibody reaction with Nesfatin,
(2) an antibody according to (1), obtainable by using a peptide having the following amino acid sequence as an immunogen:
NSF1-C18:
(SEQ ID NO: 1)
-Gly-Cys-Ser-Lys-Glu-Leu-Asp-Leu-Val-Ser-His-
His-Val-Arg-Thr-Lys-Leu-Asp-Glu-Leu,
(3) an antibody performing an antigen-antibody reaction with Nesfatin-1, but not substantially performing the antigen-antibody reaction with NucB1,
(4) a monoclonal antibody produced by a hybridoma deposited under an accession number: FERM ABP-10881, FERM ABP-10882, FERM ABP-10883 or FERM ABP-10884
(5) an antibody performing an antigen-antibody reaction with Nesfatin-1, but not substantially performing the antigen-antibody reaction with Nesfatin and NucB1, and
(6) a monoclonal antibody produced by a hybridoma deposited under an accession number: FERM ABP-10882 or FERM ABP-10884.

26. A detection kit of Nesfatin used for the detection method according to claim 23, comprising:
a labeled Nesfatin standard substance and an antibody performing an antigen-antibody reaction with Nesfatin; and
wherein the antibody is an antibody performing an antigen-antibody reaction with Nesfatin, but not substantially performing the antigen-antibody reaction with Nesfatin-1 and NucB1.

27. A pharmaceutical composition for increased appetite and/or increased body weight gain, comprising an antibody selected from the group consisting of:
(1) an antibody performing an antigen-antibody reaction with Nesfatin-1, but not substantially performing the antigen-antibody reaction with Nesfatin,
(2) an antibody according to (1), obtainable by using a peptide having the following amino acid sequence as an immunogen:
NSF1-C18:
(SEQ ID NO: 1)
-Gly-Cys-Ser-Lys-Glu-Leu-Asp-Leu-Val-Ser-His-
His-Val-Arg-Thr-Lys-Leu-Asp-Glu-Leu,
(3) an antibody performing an antigen-antibody reaction with Nesfatin-1, but not substantially performing the antigen-antibody reaction with NucB1,
(4) a monoclonal antibody produced by a hybridoma deposited under an accession number: FERM ABP-10881, FERM ABP-10882, FERM ABP-10883 or FERM ABP-10884
(5) an antibody performing an antigen-antibody reaction with Nesfatin-1, but not substantially performing the antigen-antibody reaction with Nesfatin and NucB1,
(6) a monoclonal antibody produced by a hybridoma deposited under an accession number: FERM ABP-10882 or FERM ABP-10884, and
an antibody performing an antigen-antibody reaction with Nesfatin, but not substantially performing the antigen-antibody reaction with Nesfatin-1 and NucB1.
